(19) United States
(12) Patent Application Publication
(10) Pub. No.: US 2025/0002492 A1
(43) Pub. Date: Jan. 2, 2025

(54) COMPOSITIONS OF FOLATE ABSORPTION INHIBITORS AND METHODS THEREOF

(71) Applicant: THE TEXAS A&M UNIVERSITY SYSTEM, College Station, TX (US)

(72) Inventor: Michael POLYMENIS, College Station, TX (US)

(21) Appl. No.: 18/752,069

(22) Filed: Jun. 24, 2024

Related U.S. Application Data
(60) Provisional application No. 63/522,534, filed on Jun. 22, 2023.

(51) Int. Cl. C07D 471/04; A61K 31/365; A61K 31/366; A61K 31/404; A61K 31/409; A61K 31/437; A61K 31/4433; A61K 31/506; A61K 31/5513; C07D 307/33; C07D 471/14; C07D 487/04; C07D 487/20; C07D 491/147; C07D 493/04; C07D 519/00

(52) U.S. Cl. CPC C07D 471/04; A61K 31/365; A61K 31/366; A61K 31/404; A61K 31/409; A61K 31/437; A61K 31/4433; A61K 31/506; A61K 31/5513; C07D 307/33; C07D 471/14; C07D 487/04; C07D 487/20; C07D 491/147; C07D 493/04; C07D 519/00

(57) ABSTRACT

The present disclosure relates to folate inhibitor compounds, pharmaceutical compositions containing folate inhibitor compounds, and methods of using folate inhibitor compounds to promote healthy aging.

### CROSS-REFERENCE TO RELATED APPLICATIONS

This application claims the benefit under 35 U.S.C. § 119 (e) of U.S. Provisional Application Ser. No. 63/522,534, filed on Jun. 22, 2023, the entire disclosure of which is incorporated herein by reference.

### GOVERNMENT RIGHTS

This invention was made with government support under R01GM123139 awarded by the National Institutes of Health. The government has certain rights in the invention.

### BACKGROUND

Folate is a vitamin essential for the cellular reactions that move and use single-carbon functional groups, also known as one-carbon units (“1C”). These reactions involve the metabolism of amino acids (e.g., serine, glycine, methionine, and histidine) and the synthesis of purines, thymidylate, and phospholipids. As a result, folate is vital for making new cells, for instance during fetal development. In contrast, the role of folate later in life for modulating the healthy, disease-free healthspan of individuals is poorly understood.

Most animals, including humans, cannot synthesize folate. Folate and its various forms (e.g., 5-methyl tetrahydrofolate that is absorbed through the intestinal mucosa) do not cross biological membranes but instead rely on cellular receptors that bind folate and bring it inside cells. Dietary folate is absorbed in the intestine primarily by the proton-coupled folate transporter (PCFT/SLC46A1). Other receptors can transport folate inside the cells in the various tissues of the body, including the reduced folate receptor (RFC/SLC19A1) that is ubiquitously expressed. Additional receptors, such as FR alpha and FR beta, can also bind folate and bring it inside cells by receptor-mediated endocytosis.

To help prevent congenital abnormalities, the United States and other countries have implemented large-scale folate fortification of staple foods. Concerns have been raised that mandatory folic acid fortification could negatively affect older individuals. Some observations suggested a possible link between folic acid fortification and increased colorectal cancer rates. However, later analyses did not identify specific risks from existing mandatory folic acid fortification in the general population. This conclusion neither refutes nor contradicts the idea that a moderate decrease in folic acid intake among older adults may improve healthspan. Merely because high folic acid intake does not harm the health of older adults does not negate the possibility that a lower folic acid intake might enhance health.

On the other hand, antifolate drugs have been used for decades to control excessive cell proliferation in diseases such as cancer, rheumatoid arthritis, and psoriasis. The impact of low folate status on life-long health is poorly understood.

Loss-of-function genetic interventions in 1C enzymes may promote longevity in invertebrate model systems. 1C metabolism allocates resources for biosynthesis, providing precursors for nucleotides, proteins, and lipids. Furthermore, 1C metabolism also adjusts the methylation and redox status of the cell. Lower methyl-tetrahydrofolate (methyl-THF) level is a common signature of pro-longevity pathways, and methionine restriction extends longevity in several organisms. Mutations in the large (60S) subunit ribosomal proteins constitute a significant class of pro-longevity mutations in yeast and other species. Using ribosome profiling, translational control patterns responsible for increased longevity have been identified. Long-lived mutants had significantly reduced translational efficiency of transcripts encoding enzymes of the broader network of 1C metabolism. Steady-state metabolite profiling of these mutants was consistent with changes in 1C metabolism.

Higher folate intake is beneficial in earlier stages of life when increased cell proliferation is necessary for proper development. Later in life, a lower folate intake may promote healthy aging. Notably, such differential effects could make folate interventions in aged individuals feasible and effective without impeding the benefits of high folate intake during early life.

Therefore, the discovery and development of selective inhibitors of folate intake, such as folate absorption inhibitors and folate transporter inhibitors, represents a new therapeutic goal for promoting healthy aging later in life.

### SUMMARY

The present disclosure relates to compounds that inhibit folate uptake into cells and methods thereof. In particular, the present disclosure provides selective inhibitor compounds of proton-coupled folate transporter (PCFT/SLC46A1) as well as selective inhibitor compounds of reduced folate receptor (RFC/SLC19A1) and methods of using the same.

In one aspect, the disclosure relates to a compound of Formula I,

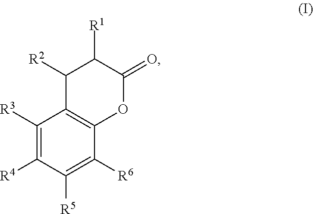

or a pharmaceutically acceptable salt thereof, wherein each of R¹, R², R³, R⁴, R⁵, and R⁶ is as described herein.

In another aspect, the disclosure relates to a compound of Formula II,

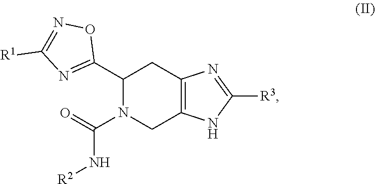

or a pharmaceutically acceptable salt thereof, wherein each of R¹, R², and R³ is as described herein.

In another aspect, the disclosure relates to a compound of Formula III,

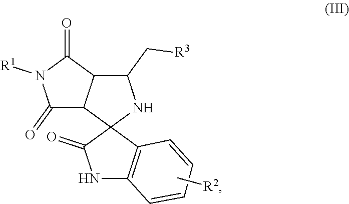

or a pharmaceutically acceptable salt thereof, wherein each of R¹, R², and R³ is as described herein.

In another aspect, the disclosure relates to a compound of Formula IV,

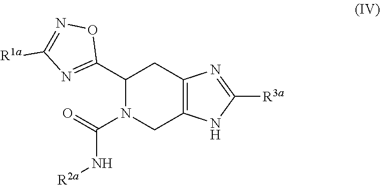

or a pharmaceutically acceptable salt thereof, wherein each of R^{1a}, R^{2a}, and R^{3a} is as described herein.

In another aspect, the disclosure relates to a compound of Formula V,

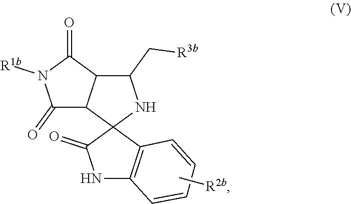

or a pharmaceutically acceptable salt thereof, wherein each of R^{1b}, R^{2b}, and R^{3b} is as described herein.

In certain embodiments of the above aspects, the compound of the disclosure is a compound of Formula I, II, III, IV, or V, or the compound is selected from those species described or exemplified in the detailed description below.

In further aspects, the disclosure relates to a pharmaceutical composition comprising at least one compound of the disclosure, such as a compound of Formula I, II, III, IV, or V or a compound selected from species described herein, or a pharmaceutically acceptable salt thereof. Pharmaceutical compositions according to the disclosure may further comprise a pharmaceutically acceptable excipient.

In further aspects, the disclosure relates to a compound of the disclosure, such as a compound of Formula I, II, III, IV, or V or a compound selected from species described herein, or a pharmaceutically acceptable salt thereof, for use as a medicament.

In further aspects, the disclosure relates to a method of inhibiting folate uptake comprising administering to a subject in need of such treatment an effective amount of at least one compound of the disclosure, such as a compound of Formula I, II, III, IV, or V or a compound selected from species described herein, or a pharmaceutically acceptable salt thereof.

In further aspects, the disclosure relates to a use of a compound of the disclosure, such as a compound of Formula I, II, III, IV, or V or a compound selected from species described herein, or a pharmaceutically acceptable salt thereof, in the preparation of a medicament.

In further aspects, the disclosure relates to a method of inhibiting folate uptake into a cell comprising contacting the cell with an effective amount of at least one compound of the disclosure, such as a compound of Formula I, II, III, IV, or V or a compound selected from species described herein, or a pharmaceutically acceptable salt thereof, and/or with at least one pharmaceutical composition of the disclosure, wherein the contacting is in vitro, ex vivo, or in vivo.

Additional embodiments, features, and advantages of the disclosure will be apparent from the following detailed description and through practice of the disclosure. The compounds of the present disclosure can be described as embodiments in any of the following enumerated clauses. It will be understood that any of the embodiments described herein can be used in connection with any other embodiments described herein to the extent that the embodiments do not contradict one another.

### BRIEF DESCRIPTION OF THE DRAWINGS

FIGS. 1A-1E show survival curves for S. cerevisiae MATα (strain BY4742) cells compared to experiment-matched cells lacking SHM1 (FIG. 1A), SHM2 (FIG. 1B), ADE17 (FIG. 1C), ADE2 (FIG. 1D), or ADE3 (FIG. 1E). Mean lifespans are shown in parentheses, along with the number of cells assayed in each case. In the case of shm1Δ, shm2Δ, ade17Δ, and ade3Δ cells, the lifespan extension was significant (p<0.0001; based on the log-rank test). Deletion of enzymes of one-carbon metabolic pathways extends replicative lifespan in yeast.

FIG. 2A shows a schematic of 1C enzymatic reactions. The reactions inhibited by methotrexate (MTX) or ATIC Dimerization Inhibitor (ATIC-Inh) are indicated.

FIG. 2B shows survival curves on rich undefined media (YPD) for S. cerevisiae MATα (strain BY4742) cells, compared to experiment-matched cells mock-treated with DMSO or three different doses of MTX (as indicated). The number of mother cell divisions (replicative lifespan) is on the x-axis.

FIG. 2C shows survival curves for C. elegans (strain N2) exposed to the indicated doses of MTX. Survival probability is on the y-axis, and time (in days) is on the x-axis. Mean lifespans and the number of animals assayed in each case are shown in parentheses. The indicated p value was based on the log-rank test.

FIG. 2D shows survival curves for C. elegans (strain N2) exposed to the indicated doses of ATIC-Inh. Survival probability is on the y-axis, and time (in days) is on the x-axis. Mean lifespans and the number of animals assayed in each case are shown in parentheses. The indicated p value was based on the log-rank test.

FIG. 3 shows cell size histograms of yeast cells (BY4742 strain background) treated with the indicated doses of methotrexate. Size (in fL) is on the x-axis, and the cell number on the y-axis.

FIG. 4 shows survival curves of female Swiss mice on chronic, low-dose methotrexate (MTX). MTX was administered in the food at the indicated dose every other week, starting at 7 weeks of age. Raw data were from (Rustia and Shubik, Life-span carcinogenicity tests with 4-amino-N10-methylpteroylglutamic acid (methotrexate) in Swiss mice and Syrian golden hamsters. 1973. Toxicol Appl Pharmacol 26:329-338). Survival probability is on the y-axis, and time (in weeks) is on the x-axis. Mean lifespans and the number of animals assayed in each case are shown in parentheses. The indicated p value was based on the log-rank test.

FIG. 5A shows a schematic of the study design.

FIG. 5B shows serum folate levels measured at 120 weeks, using an established microbiological assay and shown on the y-axis. The different diets are on the x-axis, as indicated for the folate/choline-replete (F/C+) or -limited (F/C−) groups. The boxplot graphs were generated with R language functions. Each box is drawn from the first to the third quartile, with a horizontal line denoting the median. The whiskers show the interquartile range (IQR), and they were drawn at 1.5×IQR. The replicates were all biological ones from different animals.

FIG. 5C shows the weight of the animals (y-axis) was measured every month (x-axis). The red horizontal lines were drawn to help visualize the weight changes over time in female and male mice. Loess curves and the std errors at a 0.95 level are shown. A mixed effects regression model was applied with the lme4 and lmer R language packages to evaluate the effects of the fixed variables (diet, time, sex) on the observed weight, taking into account the repeated longitudinal measurements on each mouse. A negative association with the F/C+ diet was significant (p=0.0313).

FIG. 6A shows a plot of the standardized residuals on the y-axis and the fitted values on the x-axis, from the data shown in FIG. 5C.

FIG. 6B shows a Q-Q plot of the residuals, with the sample quantiles from the measurements in FIG. 5C (y-axis), against the theoretical normally distributed ones (x-axis).

FIG. 7A shows blood cell numbers measured at 108 weeks of age from animals of the indicated sex and diet group.

FIG. 7B shows cell size (y-axis; in fL) measured from the same samples shown in FIG. 7A from animals of the indicated sex and diet group. The boxplots were drawn as in FIG. 5B.

FIG. 8A shows survival curves of male mice placed on dietary folate restriction late in life. Survival probability is on the y-axis, and time (in weeks) on the x-axis, from animals of each diet test group. The indicated p value was based on the log-rank test.

FIG. 8B shows survival curves of female mice placed on dietary folate restriction late in life. Survival probability is on the y-axis, and time (in weeks) on the x-axis, from animals of each diet test group. The indicated p value was based on the log-rank test.

FIG. 9A shows Frailty Index scores on the y-axis. Measurements were taken at the indicated times from female and male animals of each diet test group.

FIG. 9B shows the total, fat, and lean mass of each mouse in the study measured by MRI, and shown on the y-axis. The boxplots were drawn as in FIG. 5B.

FIG. 10 shows step width variance and gait symmetry values (shown on the y-axis) measured with the Digigait system. As indicated, measurements were taken at the indicated times from female and male animals of each diet test group. The boxplots were drawn as in the previous figures.

FIG. 11A shows open field assays in mice placed on dietary folate restriction late in life. The inner zone and moving times during open field evaluation are on the y-axis. The boxplots were drawn as in the previous figures.

FIG. 11B shows novel object recognition assays in mice placed on dietary folate restriction late in life. The discrimination ratio values, reflecting the ability of the mice to recognize a new object, are on the y-axis. The boxplots were drawn as in the previous figures.

FIG. 12 shows several parameters of cardiac function (x-axis) measured by echocardiography, and the corresponding values are on the y-axis. Measurements were taken at the indicated times from female and male animals of each diet test group, as indicated. The boxplots were drawn as in the previous figures.

FIG. 13A shows photographs of male mice on the indicated diet taken at 85 weeks of age. Signs of graying were visible on the coat of mice on the F/C+ diet (left) but not on the coat of mice on the F/C− diet (right).

FIG. 13B shows the respiratory exchange rate (RER) values (y-axis) from 6-8 mice in each indicated group at 108 weeks of age. The measurements were taken after the animals were acclimated for two days in the metabolic cages. The period between the vertical lines corresponds to night-time when mice are active. Loess curves and the std errors at a 0.95 level are shown.

FIG. 14A shows beta diversity principal component analysis plots were based on Bray-Curtis dissimilarity indices, of the DNA from the fecal microbiome sampled and sequenced at 90 weeks of age, from 5 mice in each test group. Three principal components (PC1,2,3; shown at the top) accounted for ˜70% of the dataset variance.

FIG. 14B shows metabolic pathway biomarker changes associated with folate limitation late in life, from metagenomic data of the fecal microbiome. The LEfSe computational pipeline was used to determine the features most likely to explain the observed differences. The computed linear discriminant analysis (LDA) scores (Log 10-transformed) are on the x-axis. LDA scores incorporate effect sizes, ranking the relevance of the identified biomarkers and enabling their visualization. Arrows indicate pathways involved in amino acid synthesis and pathways involved in IMP synthesis.

FIG. 15 shows Shannon's diversity index (y-axis) is shown for each sex and diet as a metric of the alpha diversity of the fecal microbiome sampled and sequenced at 90 weeks of age, from 5 mice in each test group. The boxplots were drawn as in the previous figures.

FIG. 16A shows steady-state serum amino acid levels were measured at 120 weeks of age using an HPLC-based assay. The measured amounts (in nmol/mL) are on the y-axis. The different diets are on the x-axis for each amino acid, as indicated. The boxplots were drawn as in the previous figures. Differences in glutamine (Gln) levels in male mice were significant (marked with an asterisk; p=2.6E-05, based on the Wilcoxon rank sum test).

FIG. 16B shows steady-state levels of primary and biogenic amine metabolites from liver tissue collected at 120 weeks of age were measured by GC-TOF MS and HILIC-QTOF MS/MS, respectively. Changes in the indicated pairwise comparisons were identified from the magnitude of the difference (x-axis; Log 2-fold change) and statistical significance (y-axis; based on robust bootstrap ANOVA tests), as indicated.

FIG. 16C shows mtabolite enrichment analysis based on the MetaboAnalyst platform for male mice from the data in FIG. 16B, for metabolites present at significantly lower levels under folate-limitation (p<0.05 and >1.5-fold change). The corresponding metabolic pathways are on the y-axis, and the p values are on the x-axis. The size of each bubble on the chart reflects the relative number of ‘hit’ metabolites in each pathway. The shade of each bubble reflects the enrichment ratio, which is the number of hits within a metabolic pathway divided by the expected number of hits. Only pathways with enrichment >2 and FDR values <0.05 are shown.

FIG. 17 shows the pathology of the indicated tissues, collected at 120 weeks of age, was scored on a 0-4 scale (with 4 reflecting the highest degree of pathological changes) and shown on the y-axis. The different diets are on the x-axis. Differences in kidney abnormalities in male mice were significant (indicated with an asterisk; p=0.0165, based on the Wilcoxon rank sum test). The boxplots were drawn as in the previous figures.

FIG. 18 shows serum cytokine levels were measured at 120 weeks of age using a mouse multiplex cytokine assay service by Eve Technologies. The measured amounts (in pg/mL; Log 10-transformed) are on the y-axis. The different diets are on the x-axis for each cytokine, as indicated. Significant differences in the measured values within a sex and diet group are indicated with an asterisk (p<0.05, based on the Wilcoxon rank sum test). The boxplots were drawn as in the previous figures.

FIG. 19A shows DNA methylation at multiple sites in the genome was measured with the epigenetic clock assay by ZymoResearch from liver samples at 120 weeks of age. Based on the measured against the predicted changes, a biological age estimate was compared to the actual age (ΔDNAage; shown on the y-axis). The different diets are on the x-axis. The boxplots were drawn as in the previous figures.

FIG. 19B shows Uracil levels in the DNA were measured from liver tissue collected at 120 weeks of age (y-axis; in pg/μg). The different diets are on the x-axis. The boxplots were drawn as in the previous figures.

FIG. 20A shows steady-state levels of mRNAs from liver tissue collected at 120 weeks of age were measured by RNAseq. Changes in the indicated pairwise comparisons were identified from the magnitude of the difference (x-axis; Log 2-fold change) and statistical significance (y-axis; based on robust bootstrap ANOVA tests), as indicated. The number of transcripts whose levels changed within these thresholds is shown in each case.

FIG. 20B shows transcript enrichment analysis based on the PANTHER platform for male mice from the data in FIG. 20A, for transcripts present at significantly lower levels under folate-limitation (p<0.05 and >1.5-fold change) that could be assigned to a specific gene ID (n=165). The corresponding biological processes are on the y-axis, and the p values are on the x-axis. The size of each bubble on the chart reflects the relative number of ‘hit’ transcripts in each process. The shade of each bubble reflects the enrichment ratio, which is the number of hits within a process divided by the expected number of hits. Only pathways with enrichment >2 and FDR values <0.05 are shown.

FIG. 20C shows transcript enrichment analysis for female mice from the data in FIG. 20A, for transcripts present at significantly lower levels under folate-limitation (p<0.05 and >1.5-fold change) that could be assigned to a specific gene ID (n=490). The plots were drawn as in FIG. 20B.

FIG. 21 shows phosphorylated RPS6 (P-RPS6) levels (y-axis) were measured with a phosphospecific antibody and normalized against the signal from an antibody against RPS6 (detecting the total amount). The different diets are on the x-axis. The boxplots were drawn as in the previous figures.

FIG. 22 shows phosphorylated 4EBP1 (P-4EBP1) levels (y-axis) were measured with a phosphospecific antibody and normalized against the signal from an antibody against 4EBP1 (detecting the total amount). The different diets are on the x-axis. The boxplots were drawn as in the previous figures.

FIG. 23 shows IGF-1 levels (in pg/mL; shown on the y-axis) were measured with a commercial mouse IGF-1 ELISA Kit. The different diets are on the x-axis. The boxplots were drawn as in the previous figures. In female mice, the difference was statistically significant, indicated with an asterisk (p=0.028, based on the Wilcoxon rank sum test).

FIG. 24 shows a schematic representation exploiting the contrasting effects of folate early vs. late in life by inhibiting the folate transporter.

FIG. 25 shows a schematic representation of the selection and testing of the compounds of the disclosure.

FIG. 26 shows survival plots of the indicated doses of compound B1. The mean lifespan, the number of animals (n) tested, and the p-value associated with the log-rank test are shown.

### DETAILED DESCRIPTION

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended clauses.

For the sake of brevity, the disclosures of the publications cited in this specification, including patents, are herein incorporated by reference. Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

As used herein and in the appended clauses, the singular forms “a,” “an,” and “the” include plural referents unless the context clearly dictates otherwise. It is further noted that the clauses may be drafted to exclude any optional element. As such, this statement is intended to serve as antecedent basis for use of such exclusive terminology as “solely,” “only” and the like in connection with the recitation of clause elements, or use of a “negative” limitation.

As used herein, the terms “including,” “containing,” and “comprising” are used in their open, non-limiting sense.

To provide a more concise description, some of the quantitative expressions given herein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

Except as otherwise noted, the methods and techniques of the present embodiments are generally performed according to conventional methods well known in the art and as described in various general and more specific references that are cited and discussed throughout the present specification. See, e.g., Loudon, Organic Chemistry, Fourth Edition, New York: Oxford University Press, 2002, pp. 360-361, 1084-1085; Smith and March, March's Advanced Organic Chemistry: Reactions, Mechanisms, and Structure, Fifth Edition, Wiley-Interscience, 2001.

Chemical nomenclature for compounds described herein has generally been derived using the commercially-available ACD/Name 2014 (ACD/Labs) or ChemBioDraw Ultra 13.0 (Perkin Elmer).

As used herein and in connection with chemical structures depicting the various embodiments described herein, “*”, “**”, and “ [symbol] ”, each represent a point of covalent attachment of the chemical group or chemical structure in which the identifier is shown to an adjacent chemical group or chemical structure. For example, in a hypothetical chemical structure A-B, where A and B are joined by a covalent bond, in some embodiments, the portion of A-B defined by the group or chemical structure A can be represented by “A-*” “A-**”, or

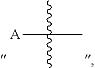

where each of “-*”, “-**”, and

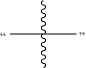

represents a bond to A and the point of covalent bond attachment to B. Alternatively, in some embodiments, the portion of A-B defined by the group or chemical structure B can be represented by “*—B”, “**—B”, or

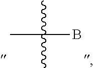

where each of “-*”, “-**”, and “

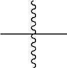

” represents a bond to B and the point of covalent bond attachment to A.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination. All combinations of the embodiments pertaining to the chemical groups represented by the variables are specifically embraced by the present disclosure and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present disclosure and are disclosed herein just as if each and every such sub-combination of chemical groups was individually and explicitly disclosed herein.

### Pharmaceutical Compositions

The compositions and methods of the present disclosure may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the disclosure and a pharmaceutically acceptable carrier.

Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the disclosure. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the disclosure. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (or preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the disclosure, with the carrier and, optionally, one or more accessory ingredients. In general, the formulations are prepared by uniformly and intimately bringing into association a compound of the present disclosure with liquid carriers, or finely divided solid carriers, or both, and then, if necessary, shaping the product.

Formulations of the disclosure suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present disclosure as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present disclosure to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this disclosure, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with a compound of the disclosure. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the disclosure will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present disclosure, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the disclosure may be used alone or conjointly administered with another type of therapeutic agent.

The present disclosure includes the use of pharmaceutically acceptable salts of compounds of the disclosure in the compositions and methods of the present disclosure. In certain embodiments, contemplated salts of the disclosure include, but are not limited to, alkyl, dialkyl, trialkyl or tetra-alkyl ammonium salts. In certain embodiments, contemplated salts of the disclosure include, but are not limited to, L-arginine, benenthamine, benzathine, betaine, calcium hydroxide, choline, deanol, diethanolamine, diethylamine, 2-(diethylamino) ethanol, ethanolamine, ethylenediamine, N-methylglucamine, hydrabamine, 1H-imidazole, lithium, L-lysine, magnesium, 4-(2-hydroxyethyl) morpholine, piperazine, potassium, 1-(2-hydroxyethyl) pyrrolidine, sodium, triethanolamine, tromethamine, and zinc salts. In certain embodiments, contemplated salts of the disclosure include, but are not limited to, Na, Ca, K, Mg, Zn or other metal salts. In certain embodiments, contemplated salts of the disclosure include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N. Y. (1999); and Gilbert et al., “Developmental Biology, 6th ed.”, Sinauer Associates, Inc., Sunderland, Mass. (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, Calif. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known. The ability of such agents to inhibit AR or promote AR degradation may render them suitable as “therapeutic agents” in the methods and compositions of this disclosure.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present disclosure can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: deuterium, hydroxyl, hydroxyalkyl, alkoxy, halogen, alkyl, alkenyl, alkynyl, nitro, silyl, acyl, acyloxy, aryl, cycloalkyl, heterocyclyl, amino, aminoalkyl, cyano, haloalkyl, haloalkoxy, haloalkenyl, haloalkynyl, ketone or oxo, carboxy, amide, ester, OCOCH₂O-alkyl, OP(O)(O-alkyl)₂, or CH₂OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. Cₒ alkyl indicates a hydrogen where the group is in a terminal position, a bond if internal. A C₁₋₆ alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS-.

The term “amide”, as used herein, refers to a group

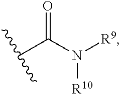

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

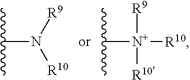

wherein R⁹, R¹⁰, and R¹⁰, each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

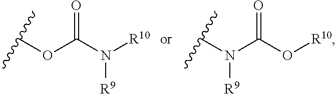

wherein R⁹ and R¹⁰ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo [2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbonate” is art-recognized and refers to a group —OCO₂—.

The term “carboxy”, as used herein, refers to a group represented by the formula —CO₂H.

The term “cycloalkyl” refers to a saturated or partially saturated, monocyclic or polycyclic mono-valent carbocycle. The term “cycloalkylene” refers to a saturated or partially saturated, monocyclic or polycyclic di-valent carbocycle. In some embodiments, it can be advantageous to limit the number of atoms in a “cycloalkyl” or “cycloalkylene” to a specific range of atoms, such as having 3 to 12 ring atoms. Polycyclic carbocycles include fused, bridged, and spiro polycyclic systems. Illustrative examples of cycloalkyl groups include mono-valent radicals of the following entities, while cycloalkylene groups include di-valent radicals of the following entities, in the form of properly bonded moieties:

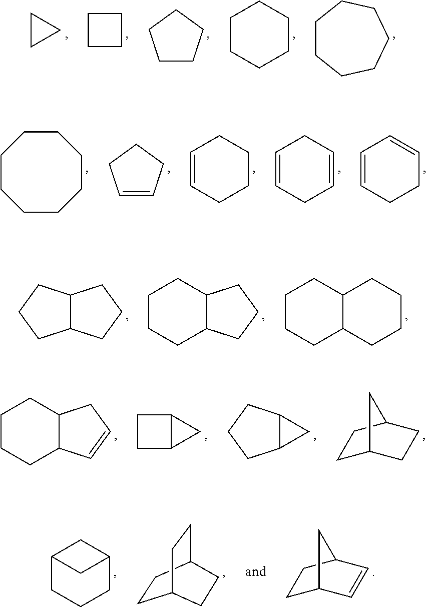

It will be appreciated that a cycloalkyl or cycloalkylene group can be unsubstituted or substituted as described herein. A cycloalkyl or cycloalkylene group can be substituted with any of the substituents in the various embodiments described herein, including one or more of such substituents.

The term “ester”, as used herein, refers to a group —C(O)OR⁸ wherein R⁸ represents a hydrocarbyl group.

The term “ketone”, as used herein, refers to a group —C(O)R⁷ wherein R⁷ represents a hydrocarbyl group (e.g., alkyl, aryl, heteroaryl).

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrone, benzopyrone (e.g., chromone), pyrrole, benzopyrrole, furan, benzofuran, thiophene, imidazole, oxazole, thiazole, indole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, benzopyran (chromene), benzodihydropyran (chromane), dihydrobenzodioxine (benzodioxan), dihydrobenzofuran, benzodioxole, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

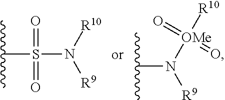

wherein R⁹ and R¹⁰ independently represents hydrogen or hydrocarbyl.

The term “sulfoxide” is art-recognized and refers to the group —S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁸ or —SC(O)R⁸ wherein R⁸ represents a hydrocarbyl.

The term “thioether”, as used herein, is equivalent to an ether, wherein the oxygen is replaced with a sulfur.

The term “urea” is art-recognized and may be represented by the general formula

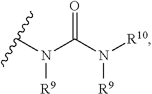

wherein R⁹ and R¹⁰ independently represent hydrogen or a hydrocarbyl.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by a compound of the disclosure. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of the disclosure for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by a compound of the disclosure or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). Sec, e.g., WO 01/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form the compound of the present disclosure (e.g., compounds of formula I). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce a compound of Formula I. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “Log of solubility”, “Log S” or “log S” as used herein is used in the art to quantify the aqueous solubility of a compound. The aqueous solubility of a compound significantly affects its absorption and distribution characteristics. A low solubility often goes along with a poor absorption. Log S value is a unit stripped logarithm (base 10) of the solubility measured in mol/liter.

### Representative Embodiments

In some embodiments, the disclosure relates to a compound of Formula I,

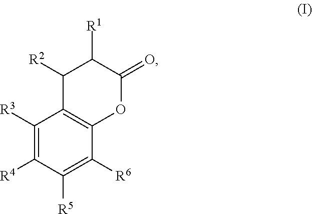

or a pharmaceutically acceptable salt thereof, wherein:
each of R¹ and R⁶ is H;
R² is substituted or unsubstituted carbocyclyl, heterocyclyl, aryl, or heteroaryl; and
each of R³, R⁴, and R⁵ is independently H, deuterium, hydroxy, alkoxy, alkyl, alkenyl, alkynyl, carboxy, ketone, ester, or amide; or two of R³, R⁴, and R⁵ taken together on the carbon to which they are attached form substituted or unsubstituted carbocyclyl, heterocycloalkyl, aryl, or heteroaryl.

In some embodiments, the disclosure relates to a compound of Formula Ia,

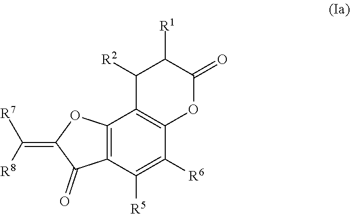

or a pharmaceutically acceptable salt thereof, wherein:
each of R¹, R⁵, and R⁶ is H;
R² is substituted or unsubstituted carbocyclyl, heterocycloalkyl, aryl, heteroaryl; and
each of R⁷ and R⁸ is independently H, or substituted or unsubstituted carbocyclyl, heterocyclyl, aryl, heteroaryl.

In some embodiments, the disclosure relates to a compound of Formula Ib,

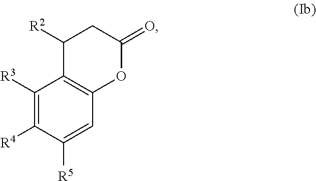

or a pharmaceutically acceptable salt thereof, wherein:
R² is a substituted or unsubstituted carbocyclyl, heterocyclyl, aryl, or heteroaryl;
one of R³ and R⁵ is H, deuterium, hydroxy, alkoxy, alkyl, alkenyl, alkynyl, carboxy, ketone, ester, or amide; and
R⁴ and the other of R³ or R⁵ are taken together on the carbon to which they are attached to form a substituted or unsubstituted carbocyclyl, heterocycloalkyl, aryl, or heteroaryl.

In some embodiments, R² is a substituted or unsubstituted carbocyclyl (e.g., C₃-C₁₂ cycloalkyl), substituted or unsubstituted heterocyclyl (e.g., 3- to 12-membered heterocyclyl), substituted or unsubstituted aryl (e.g., C₆-C₁₀ aryl), or substituted or unsubstituted heteroaryl (e.g., 5- to 12-membered heteroaryl). In some embodiments, R² is a substituted or unsubstituted heterocyclyl (e.g., 3- to 12-membered heterocyclyl), substituted or unsubstituted aryl (e.g., C₆-C₁₀ aryl), or substituted or unsubstituted heteroaryl (e.g., 5- to 12-membered heteroaryl). In some embodiments, R² is a substituted or unsubstituted aryl (e.g., C₆-C₁₀ aryl), or a substituted or unsubstituted heteroaryl (e.g., 5- to 12-membered heteroaryl).

In some embodiments, R² is a substituted or unsubstituted aryl (e.g., C₆-C₁₀ aryl). In some embodiments, R² is unsubstituted C₆-C₁₀ aryl or C₆-C₁₀ aryl substituted with deuterium, hydroxyl, halo, alkoxy (e.g., OC₁-C₆ alkyl), alkyl (e.g., C₁-C₆ alkyl), alkenyl (e.g., C₂-C₆ alkenyl), alkynyl (e.g., C₂-C₆ alkynyl), carboxy, ketone, ester, or amide. In some embodiments, R² is aryl (e.g., C₆-C₁₀ aryl) substituted with deuterium, hydroxyl, halo, alkoxy (e.g., OC₁-C₆ alkyl), alkyl (e.g., C₁-C₆ alkyl), alkenyl (e.g., C₂-C₆ alkenyl), alkynyl (e.g., C₂-C₆ alkynyl), carboxy, ketone, ester, or amide. In some embodiments, R² is a substituted or unsubstituted phenyl. In some embodiments, R² is unsubstituted phenyl or phenyl substituted with deuterium, hydroxyl, halo, alkoxy (e.g., OC₁-C₆ alkyl), alkyl (e.g., C₁-C₆ alkyl), alkenyl (e.g., C₂-C₆ alkenyl), alkynyl (e.g., C₂-C₆ alkynyl), carboxy, ketone, ester, or amide. In some embodiments, R² is phenyl substituted with deuterium, hydroxyl, halo, alkoxy (e.g., OC₁-C₆ alkyl), alkyl (e.g., C₁-C₆ alkyl), alkenyl (e.g., C₂-C₆ alkenyl), alkynyl (e.g., C₂-C₆ alkynyl), carboxy, ketone, ester, or amide. In some embodiments, R² is carboxyphenyl.

In some embodiments, R² is a substituted or unsubstituted heterocyclyl (e.g., 3- to 12-membered heterocyclyl). In some embodiments, R² is unsubstituted 3- to 12-membered heterocyclyl or 3- to 12-membered heterocyclyl substituted with deuterium, hydroxyl, halo, alkoxy, alkyl, alkenyl, alkynyl, carboxy, ketone, ester, or amide. In some embodiments, R² is unsubstituted 3- to 12-membered heterocyclyl. In some embodiments, R² is a substituted or unsubstituted benzopyran, benzodihydropyran, benzodioxan, dihydrobenzofuran, or benzodioxole. In some embodiments, R² is benzopyran, benzodihydropyran, benzodioxan, dihydrobenzofuran, or benzodioxole, or benzopyran, benzodihydropyran, benzodioxan, dihydrobenzofuran, or benzodioxole substituted with deuterium, hydroxyl, halo, alkoxy (e.g., OC₁-C₆ alkyl), alkyl (e.g., C₁-C₆ alkyl), alkenyl (e.g., C₂-C₆ alkenyl), alkynyl (e.g., C₂-C₆ alkynyl), carboxy, ketone, ester, or amide. In some embodiments, R² is unsubstituted benzopyran, benzodihydropyran, benzodioxan, dihydrobenzofuran, or benzodioxole. In some embodiments, R² is unsubstituted benzopyran, benzodioxan, or benzodioxole.

In some embodiments, R² is a substituted or unsubstituted heteroaryl (e.g., 5- to 12-membered heteroaryl). In some embodiments, R² is unsubstituted 5- to 12-membered heteroaryl or 5- to 12-membered heteroaryl substituted with deuterium, hydroxyl, halo, alkoxy, alkyl, alkenyl, alkynyl, carboxy, ketone, ester, or amide. In some embodiments, R² is heteroaryl (e.g., 5- to 12-membered heteroaryl) substituted with deuterium, hydroxyl, halo, alkoxy (e.g., OC₁-C₆ alkyl), alkyl (e.g., C₁-C₆ alkyl), alkenyl (e.g., C₂-C₆ alkenyl), alkynyl (e.g., C₂-C₆ alkynyl), carboxy, ketone, ester, or amide. In some embodiments, R² is unsubstituted 3- to 12-membered heteroaryl. In some embodiments, R² is a substituted or unsubstituted benzopyrone. In some embodiments, R² is unsubstituted benzopyrone or benzopyrone substituted with deuterium, hydroxyl, halo, alkoxy (e.g., OC₁-C₆ alkyl), alkyl (e.g., C₁-C₆ alkyl), alkenyl (e.g., C₂-C₆ alkenyl), alkynyl (e.g., C₂-C₆ alkynyl), carboxy, ketone, ester, or amide. In some embodiments, R² is unsubstituted benzopyrone. In some embodiments, R² is benzopyrone substituted with deuterium, hydroxyl, halo, alkoxy (e.g., OC₁-C₆ alkyl), alkyl (e.g., C₁-C₆ alkyl), alkenyl (e.g., C₂-C₆ alkenyl), alkynyl (e.g., C₂-C₆ alkynyl), carboxy, ketone, ester, or amide. In some embodiments, R² is benzopyrone or methylbenzopyrone.

In some embodiments, R² is selected from the group consisting of

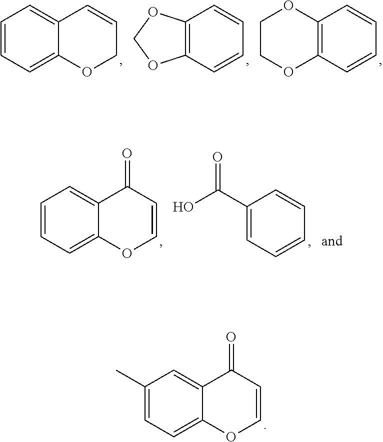

In some embodiments, R² is selected from the group consisting of

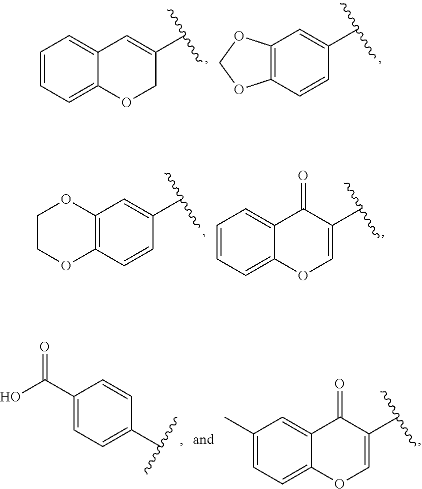

wherein “ [symbol] ” represents a point of attachment to the rest of the molecule.

In some embodiments, R³ is H, deuterium, hydroxy, alkoxy (e.g., OC₁-C₆ alkyl), alkyl (e.g., C₁-C₆ alkyl), alkenyl (e.g., C₂-C₆ alkenyl), alkynyl (e.g., C₂-C₆ alkynyl), carboxy, ketone, ester, or amide. In some embodiments, R³ is hydroxy. In some embodiments, R³ is hydroxy and R⁴ and R⁵ are taken together on the carbon to which they are attached form a substituted or unsubstituted carbocyclyl (e.g., C₃-C₁₂ cycloalkyl), substituted or unsubstituted heterocyclyl (e.g., 6- to 16-membered heterocyclyl), substituted or unsubstituted aryl (e.g., C₆-C₁₀ aryl), or substituted or unsubstituted heteroaryl (e.g., 5- to 12-membered heteroaryl). In some embodiments, R³ is hydroxy and R⁴ and R⁵ are taken together on the carbon to which they are attached form a substituted or unsubstituted heterocyclyl (e.g., 6- to 16-membered heterocyclyl). In some embodiments, R³ is hydroxy and R⁴ and R⁵ are taken together on the carbon to which they are attached form

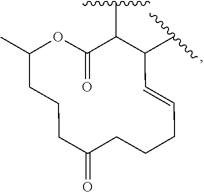 [SMILES: *C1/C=C/CCCC(=O)CCCC(C)OC(=O)C1*]

wherein “ [symbol] ” represents a point of attachment to the rest of the molecule.

In some embodiments, R³ and R⁴ are taken together on the carbon to which they are attached to form

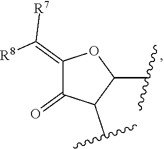

wherein “ [symbol] ” represents a point of attachment to the rest of the molecule.

In some embodiments, R⁴ and R⁵ are taken together on the carbon to which they are attached form a substituted or unsubstituted carbocyclyl (e.g., C₃-C₁₂ cycloalkyl), substituted or unsubstituted heterocyclyl (e.g., 3- to 16-membered heterocyclyl), substituted or unsubstituted aryl (e.g., C₆-C₁₀ aryl), or substituted or unsubstituted heteroaryl (e.g., 5- to 12-membered heteroaryl). In some embodiments, R⁴ and R⁵ are taken together on the carbon to which they are attached form a substituted or unsubstituted heterocyclyl (e.g., 3- to 16-membered heterocyclyl). In some embodiments, R⁴ and R⁵ are taken together on the carbon to which they are attached form

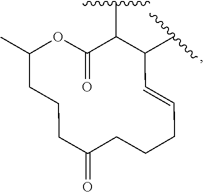 [SMILES: *C1/C=C/CCCC(=O)CCCC(C)OC(=O)C1*]

wherein “ [symbol] ” represents a point of attachment to the rest of the molecule.

In some embodiments, each of R⁷ and R⁸ is independently H, or substituted or unsubstituted carbocyclyl, heterocyclyl, aryl, heteroaryl. In some embodiments, one of R⁷ and R⁸ is H.

In some embodiments, R⁷ and R⁸ are independently selected from the group consisting of H,

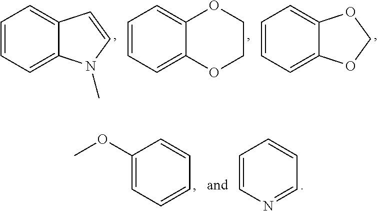

In some embodiments, R⁷ and R⁸ are independently selected from the group consisting of H,

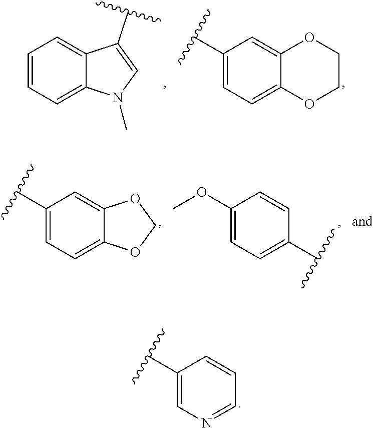

In some embodiments, one of R⁷ and R⁸ is H, and the other of R⁷ and R⁸ is selected from the group consisting of

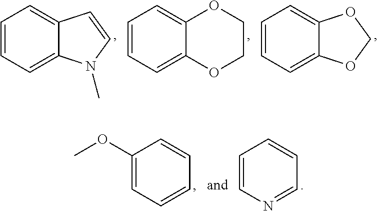

In some embodiments, one of R⁷ and R⁸ is H, and the other of R⁷ and R⁸ is selected from the group consisting of

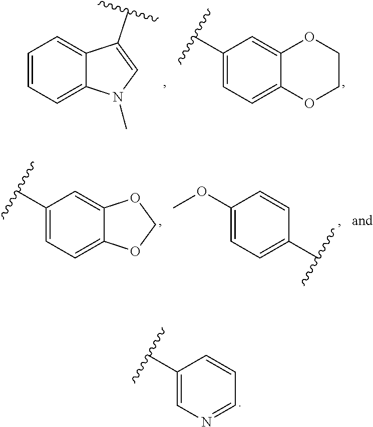

In some embodiments, the disclosure relates to a compound of Formula II,

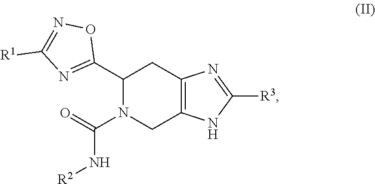

or a pharmaceutically acceptable salt thereof, wherein:
each of R¹ and R² is independently substituted or unsubstituted carbocyclyl, heterocyclyl, aryl, or heteroaryl; and
R³ is H.

In some embodiments of compounds of Formula II, R¹ is R^{1a}, R² is R^{2a}, and R³ is R^{3a}. In some embodiments of compounds of Formula II, R¹ (or R^{1a}) is

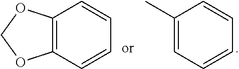

In some embodiments of compounds of Formula II, R² (or R^{2a}) is

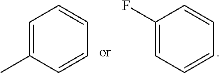

In some embodiments, the disclosure relates to a compound of Formula IV,

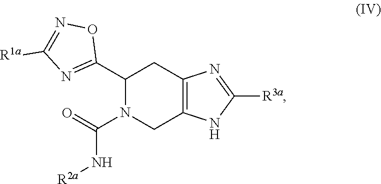

or a pharmaceutically acceptable salt thereof, wherein:
each of R^{1a} and R^{2a} is independently substituted or unsubstituted carbocyclyl, heterocyclyl, aryl, or heteroaryl; and
R^{3a} is H.

In some embodiments, the disclosure relates to a compound of Formula IIa,

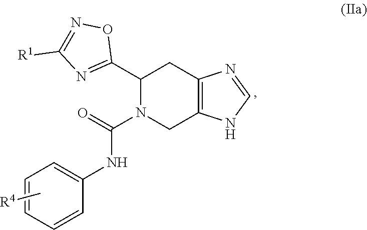

or a pharmaceutically acceptable salt thereof, wherein:
R¹ is substituted or unsubstituted carbocyclyl, heterocyclyl, aryl, or heteroaryl; and
each R⁴ is independently H, deuterium, hydroxyl, halo, alkoxy, alkyl, alkenyl, alkynyl, carboxy, ketone, ester, or amide.

In some embodiments of compounds of Formula IIa, R¹ is R^{1a} and R⁴ is R^{4a}.

In some embodiments, the disclosure relates to a compound of Formula IVa,

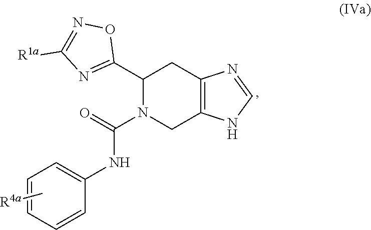

or a pharmaceutically acceptable salt thereof, wherein:
R^{1a} is substituted or unsubstituted carbocyclyl, heterocyclyl, aryl, or heteroaryl; and

each R^{4a} is independently H, deuterium, hydroxyl, halo, alkoxy, alkyl, alkenyl, alkynyl, carboxy, ketone, ester, or amide.

In some embodiments, each of R^{1a} and R^{2a} is independently a substituted or unsubstituted carbocyclyl (e.g., C₃-C₁₂ cycloalkyl), substituted or unsubstituted heterocyclyl (e.g., 3- to 12-membered heterocyclyl), substituted or unsubstituted aryl (e.g., C₆-C₁₀ aryl), or substituted or unsubstituted heteroaryl (e.g., 5- to 12-membered heteroaryl). In some embodiments, each of R^{1a} and R^{2a} is independently a substituted or unsubstituted heterocyclyl (e.g., 3- to 12-membered heterocyclyl), substituted or unsubstituted aryl (e.g., C₆-C₁₀ aryl), or substituted or unsubstituted heteroaryl (e.g., 5- to 12-membered heteroaryl).

In some embodiments, R^{1a} is a substituted or unsubstituted carbocyclyl (e.g., C₃-C₁₂ cycloalkyl), substituted or unsubstituted heterocyclyl (e.g., 3- to 12-membered heterocyclyl), substituted or unsubstituted aryl (e.g., C₆-C₁₀ aryl), or substituted or unsubstituted heteroaryl (e.g., 5- to 12-membered heteroaryl). In some embodiments, R^{1a} is a substituted or unsubstituted heterocyclyl (e.g., 3- to 12-membered heterocyclyl), substituted or unsubstituted aryl (e.g., C₆-C₁₀ aryl), or substituted or unsubstituted heteroaryl (e.g., 5- to 12-membered heteroaryl). In some embodiments, R^{1a} is a substituted or unsubstituted aryl (e.g., C₆-C₁₀ aryl), or a substituted or unsubstituted heterocyclyl (e.g., 3- to 12-membered heterocyclyl).

In some embodiments, R^{1a} is a substituted or unsubstituted aryl (e.g., C₆-C₁₀ aryl). In some embodiments, R^{1a} is unsubstituted C₆-C₁₀ aryl or C₆-C₁₀ aryl substituted with deuterium, hydroxyl, halo, alkoxy, alkyl, alkenyl, alkynyl, carboxy, ketone, ester, or amide. In some embodiments, R^{1a} is aryl (e.g., C₆-C₁₀ aryl) substituted with deuterium, hydroxyl, halo alkoxy (e.g., OC₁-C₆ alkyl), alkyl (e.g., C₁-C₆ alkyl), alkenyl (e.g., C₂-C₆ alkenyl), alkynyl (e.g., C₂-C₆ alkynyl), carboxy, ketone, ester, or amide. In some embodiments, R^{1a} is a substituted or unsubstituted phenyl. In some embodiments, R^{1a} is unsubstituted phenyl or phenyl substituted with deuterium, hydroxyl, halo, alkoxy (e.g., OC₁-C₆ alkyl), alkyl (e.g., C₁-C₆ alkyl), alkenyl (e.g., C₂-C₆ alkenyl), alkynyl (e.g., C₂-C₆ alkynyl), carboxy, ketone, ester, or amide. In some embodiments, R^{1a} is phenyl substituted with deuterium, hydroxyl, halo, alkoxy, alkyl, alkenyl, alkynyl, carboxy, ketone, ester, or amide. In some embodiments, R^{1a} is methylphenyl. In some embodiments, R^{1a} is 3-methylphenyl.

In some embodiments, R^{1a} is a substituted or unsubstituted heterocyclyl (e.g., 3- to 12-membered heterocyclyl). In some embodiments, R^{1a} is unsubstituted 3- to 12-membered heterocyclyl or 3- to 12-membered heterocyclyl substituted with deuterium, hydroxyl, halo, alkoxy (e.g., OC₁-C₆ alkyl), alkyl (e.g., C₁-C₆ alkyl), alkenyl (e.g., C₂-C₆ alkenyl), alkynyl (e.g., C₂-C₆ alkynyl), carboxy, ketone, ester, or amide. In some embodiments, R^{1a} is unsubstituted 3- to 12-membered heterocyclyl. In some embodiments, R^{1a} is a substituted or unsubstituted benzopyran, benzodihydropyran, benzodioxan, dihydrobenzofuran, or benzodioxole. In some embodiments, R^{1a} is benzopyran, benzodihydropyran, benzodioxan, dihydrobenzofuran, or benzodioxole, or benzopyran, benzodihydropyran, benzodioxan, dihydrobenzofuran, or benzodioxole substituted with deuterium, hydroxyl, halo, alkoxy, alkyl, alkenyl, alkynyl, carboxy, ketone, ester, or amide. In some embodiments, R^{1a} is unsubstituted benzopyran, benzodihydropyran, benzodioxan, dihydrobenzofuran, or benzodioxole. In some embodiments, R^{1a} is unsubstituted benzodioxole.

In some embodiments, R^{1a} is a substituted or unsubstituted heteroaryl (e.g., 5- to 12-membered heteroaryl). In some embodiments, R^{1a} is unsubstituted 5- to 12-membered heteroaryl or 5- to 12-membered heteroaryl substituted with deuterium, hydroxyl, halo, alkoxy, alkyl, alkenyl, alkynyl, carboxy, ketone, ester, or amide. In some embodiments, R^{1a} is heteroaryl (e.g., 5- to 12-membered heteroaryl) substituted with deuterium, hydroxyl, halo, alkoxy (e.g., OC₁-C₆ alkyl), alkyl (e.g., C₁-C₆ alkyl), alkenyl (e.g., C₂-C₆ alkenyl), alkynyl (e.g., C₂-C₆ alkynyl), carboxy, ketone, ester, or amide. In some embodiments, R^{1a} is unsubstituted 3- to 12-membered heteroaryl.

In some embodiments, R^{1a} is

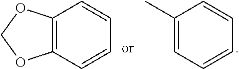

In some embodiments, R^{1a} is

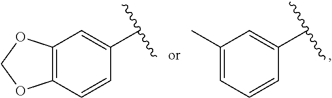

wherein “ [symbol] ” represents a point of attachment to the rest of the molecule.

In some embodiments, R^{2a} is a substituted or unsubstituted carbocyclyl (e.g., C₃-C₁₂ cycloalkyl), substituted or unsubstituted heterocyclyl (e.g., 3- to 12-membered heterocyclyl), substituted or unsubstituted aryl (e.g., C₆-C₁₀ aryl), or substituted or unsubstituted heteroaryl (e.g., 5- to 12-membered heteroaryl). In some embodiments, R^{2a} is a substituted or unsubstituted heterocyclyl (e.g., 3- to 12-membered heterocyclyl), substituted or unsubstituted aryl (e.g., C₆-C₁₀ aryl), or substituted or unsubstituted heteroaryl (e.g., 5- to 12-membered heteroaryl). In some embodiments, R^{2a} is a substituted or unsubstituted aryl (e.g., C₆-C₁₀ aryl).

In some embodiments, R^{2a} is a substituted or unsubstituted aryl (e.g., C₆-C₁₀ aryl). In some embodiments, R^{2a} is unsubstituted C₆-C₁₀ aryl or C₆-C₁₀ aryl substituted with deuterium, hydroxyl, halo, alkoxy, alkyl, alkenyl, alkynyl, carboxy, ketone, ester, or amide. In some embodiments, R^{2a} is aryl (e.g., C₆-C₁₀ aryl) substituted with deuterium, hydroxyl, halo, alkoxy (e.g., OC₁-C₆ alkyl), alkyl (e.g., C₁-C₆ alkyl), alkenyl (e.g., C₂-C₆ alkenyl), alkynyl (e.g., C₂-C₆ alkynyl), carboxy, ketone, ester, or amide. In some embodiments, R^{2a} is a substituted or unsubstituted phenyl. In some embodiments, R^{2a} is unsubstituted phenyl or phenyl substituted with deuterium, hydroxyl, halo, alkoxy (e.g., OC₁-C₆ alkyl), alkyl (e.g., C₁-C₆ alkyl), alkenyl (e.g., C₂-C₆ alkenyl), alkynyl (e.g., C₂-C₆ alkynyl), carboxy, ketone, ester, or amide. In some embodiments, R^{2a} is phenyl substituted with deuterium, hydroxyl, halo, alkoxy (e.g., OC₁-C₆ alkyl), alkyl (e.g., C₁-C₆ alkyl), alkenyl (e.g., C₂-C₆ alkenyl), alkynyl (e.g., C₂-C₆ alkynyl), carboxy, ketone, ester, or amide. In some embodiments, R^{2a} is methylphenyl or fluorophenyl. In some embodiments, R^{2a} is 4-methylphenyl. In some embodiments, R^{2a} is 3-fluorophenyl.

In some embodiments, R^{2a} is a substituted or unsubstituted heterocyclyl (e.g., 3- to 12-membered heterocyclyl). In some embodiments, R^{2a} is unsubstituted 3- to 12-membered heterocyclyl or 3- to 12-membered heterocyclyl substituted with deuterium, hydroxyl, halo, alkoxy (e.g., OC₁-C₆ alkyl), alkyl (e.g., C₁-C₆ alkyl), alkenyl (e.g., C₂-C₆ alkenyl), alkynyl (e.g., C₂-C₆ alkynyl), carboxy, ketone, ester, or amide. In some embodiments, R^{2a} is unsubstituted 3- to 12-membered heterocyclyl.

In some embodiments, R^{2a} is a substituted or unsubstituted heteroaryl (e.g., 5- to 12-membered heteroaryl). In some embodiments, R^{2a} is unsubstituted 5- to 12-membered heteroaryl or 5- to 12-membered heteroaryl substituted with deuterium, hydroxyl, halo, alkoxy (e.g., OC₁-C₆ alkyl), alkyl (e.g., C₁-C₆ alkyl), alkenyl (e.g., C₂-C₆ alkenyl), alkynyl (e.g., C₂-C₆ alkynyl), carboxy, ketone, ester, or amide. In some embodiments, R^{2a} is heteroaryl (e.g., 5- to 12-membered heteroaryl) substituted with deuterium, hydroxyl, halo, alkoxy (e.g., OC₁-C₆ alkyl), alkyl (e.g., C₁-C₆ alkyl), alkenyl (e.g., C₂-C₆ alkenyl), alkynyl (e.g., C₂-C₆ alkynyl), carboxy, ketone, ester, or amide. In some embodiments, R^{2a} is unsubstituted 3- to 12-membered heteroaryl.

In some embodiments of compounds of Formula II, R^{2a} is

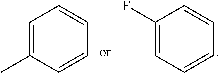

In some embodiments, R^{2a} is

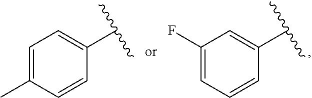

wherein “ [symbol] ” represents a point of attachment to the rest of the molecule.

In some embodiments, R³ª is H.

In some embodiments, R^{4a} (e.g., R⁴) is H, deuterium, hydroxyl, halo, alkoxy (e.g., OC₁-C₆ alkyl), alkyl (e.g., C₁-C₆ alkyl), alkenyl (e.g., C₂-C₆ alkenyl), alkynyl (e.g., C₂-C₆ alkynyl), carboxy, ketone, ester, or amide. In some embodiments, R^{4a} (e.g., R⁴) is deuterium, hydroxyl, halo, alkoxy (e.g., OC₁-C₆ alkyl), alkyl (e.g., C₁-C₆ alkyl), alkenyl (e.g., C₂-C₆ alkenyl), alkynyl (e.g., C₂-C₆ alkynyl), carboxy, ketone, ester, or amide. In some embodiments, R^{4a} is deuterium, hydroxyl, halo, alkoxy (e.g., OC₁-C₆ alkyl) or alkyl (e.g., C₁-C₆ alkyl). In some embodiments, R^{4a} is halo (e.g., fluoro) or alkyl (e.g., C₁-C₆ alkyl). In some embodiments, R^{4a} is fluoro or C₁-C₆ alkyl (e.g., methyl).

In some embodiments, the disclosure relates to a compound of Formula III,

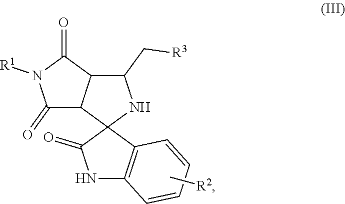

or a pharmaceutically acceptable salt thereof, wherein:
each of R¹ and R³ is independently substituted or unsubstituted carbocyclyl, heterocyclyl, aryl, or heteroaryl; and
each R² is independently H, deuterium, hydroxyl, halo, alkoxy, alkyl, alkenyl, alkynyl, carboxy, ketone, ester, or amide.

In some embodiments of compounds of Formula III, R¹ is R^{1b}, R² is R^{2b}, and R³ is R^{3b}.

In some embodiments, the disclosure relates to a compound of Formula V,

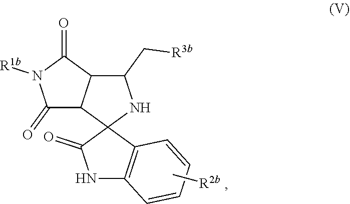

or a pharmaceutically acceptable salt thereof, wherein:
each of R^{1b} and R^{3b} is independently substituted or unsubstituted carbocyclyl, heterocyclyl, aryl, or heteroaryl; and
each R^{2b} is independently H, deuterium, hydroxyl, halo, alkoxy, alkyl, alkenyl, alkynyl, carboxy, ketone, ester, or amide.

In some embodiments, the disclosure relates to a compound of Formula IIIa,

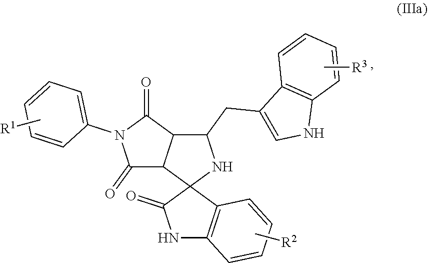

or a pharmaceutically acceptable salt thereof, wherein
each of R¹, R², and R³ is independently H, deuterium, hydroxyl, halo, alkoxy, alkyl, alkenyl, alkynyl, carboxy, ketone, ester, or amide.

In some embodiments of compounds of Formula IIIa, R¹ is R^{4b}, R² is R^{2b}, and R³ is R^{5b}.

In some embodiments, the disclosure relates to a compound of Formula Va,

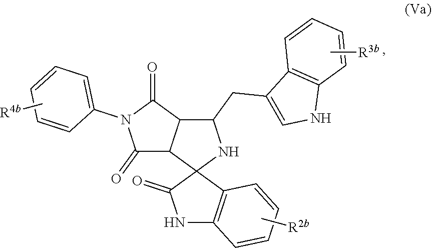

or a pharmaceutically acceptable salt thereof, wherein
each of R^{2b}, R^{4b}, and R^{5b} is independently H, deuterium, hydroxyl, halo, alkoxy, alkyl, alkenyl, alkynyl, carboxy, ketone, ester, or amide.

In some embodiments, each of R^{1b} and R^{3b} is independently a substituted or unsubstituted carbocyclyl (e.g., C₃-C₁₂ cycloalkyl), substituted or unsubstituted heterocyclyl (e.g., 3- to 12-membered heterocyclyl), substituted or unsubstituted aryl (e.g., C₆-C₁₀ aryl), or substituted or unsubstituted heteroaryl (e.g., 5- to 12-membered heteroaryl). In some embodiments, each of R^{1b} and R^{3b} is independently a substituted or unsubstituted heterocyclyl (e.g., 3- to 12-membered heterocyclyl), substituted or unsubstituted aryl (e.g., C₆-C₁₀ aryl), or substituted or unsubstituted heteroaryl (e.g., 5- to 12-membered heteroaryl).

In some embodiments, Rib is a substituted or unsubstituted carbocyclyl (e.g., C₃-C₁₂ cycloalkyl), substituted or unsubstituted heterocyclyl (e.g., 3- to 12-membered heterocyclyl), substituted or unsubstituted aryl (e.g., C₆-C₁₀ aryl), or substituted or unsubstituted heteroaryl (e.g., 5- to 12-membered heteroaryl). In some embodiments, Rib is a substituted or unsubstituted heterocyclyl (e.g., 3- to 12-membered heterocyclyl), substituted or unsubstituted aryl (e.g., C₆-C₁₀ aryl), or substituted or unsubstituted heteroaryl (e.g., 5- to 12-membered heteroaryl). In some embodiments, R^{1b} is a substituted or unsubstituted aryl (e.g., C₆-C₁₀ aryl).

In some embodiments, R^{1b} is a substituted or unsubstituted aryl (e.g., C₆-C₁₀ aryl). In some embodiments, R^{1b} is unsubstituted C₆-C₁₀ aryl or C₆-C₁₀ aryl substituted with deuterium, hydroxyl, halo, alkoxy, alkyl, alkenyl, alkynyl, carboxy, ketone, ester, or amide. In some embodiments, R^{1b} is aryl (e.g., C₆-C₁₀ aryl) substituted with deuterium, hydroxyl, halo alkoxy (e.g., OC₁-C₆ alkyl), alkyl (e.g., C₁-C₆ alkyl), alkenyl (e.g., C₂-C₆ alkenyl), alkynyl (e.g., C₂-C₆ alkynyl), carboxy, ketone, ester, or amide. In some embodiments, Rib is a substituted or unsubstituted phenyl. In some embodiments, R^{1b} is unsubstituted phenyl or phenyl substituted with deuterium, hydroxyl, halo, alkoxy (e.g., OC₁-C₆ alkyl), alkyl (e.g., C₁-C₆ alkyl), alkenyl (e.g., C₂-C₆ alkenyl), alkynyl (e.g., C₂-C₆ alkynyl), carboxy, ketone, ester, or amide. In some embodiments, Rib is phenyl substituted with deuterium, hydroxyl, halo (e.g., chloro), alkoxy, alkyl, alkenyl, alkynyl, carboxy, ketone, ester, or amide. In some embodiments, Rib is unsubstituted phenyl or chlorophenyl. In some embodiments, Rib is unsubstituted phenyl. In some embodiments, Rib is 4-chlorophenyl.

In some embodiments, Rib is a substituted or unsubstituted heterocyclyl (e.g., 3- to 12-membered heterocyclyl). In some embodiments, Rib is unsubstituted 3- to 12-membered heterocyclyl or 3- to 12-membered heterocyclyl substituted with deuterium, hydroxyl, halo, alkoxy (e.g., OC₁-C₆ alkyl), alkyl (e.g., C₁-C₆ alkyl), alkenyl (e.g., C₂-C₆ alkenyl), alkynyl (e.g., C₂-C₆ alkynyl), carboxy, ketone, ester, or amide. In some embodiments, Rib is unsubstituted 3- to 12-membered heterocyclyl.

In some embodiments, Rib is a substituted or unsubstituted heteroaryl (e.g., 5- to 12-membered heteroaryl). In some embodiments, Rib is unsubstituted 5- to 12-membered heteroaryl or 5- to 12-membered heteroaryl substituted with deuterium, hydroxyl, halo, alkoxy, alkyl, alkenyl, alkynyl, carboxy, ketone, ester, or amide. In some embodiments, R^{1b} is heteroaryl (e.g., 5- to 12-membered heteroaryl) substituted with deuterium, hydroxyl, halo, alkoxy (e.g., OC₁-C₆ alkyl), alkyl (e.g., C₁-C₆ alkyl), alkenyl (e.g., C₂-C₆ alkenyl), alkynyl (e.g., C₂-C₆ alkynyl), carboxy, ketone, ester, or amide. In some embodiments, R^{1b} is unsubstituted 3- to 12-membered heteroaryl.

In some embodiments, R^{2b} (e.g., R²) is H, deuterium, hydroxyl, halo, alkoxy (e.g., OC₁-C₆ alkyl), alkyl (e.g., C₁-C₆ alkyl), alkenyl (e.g., C₂-C₆ alkenyl), alkynyl (e.g., C₂-C₆ alkynyl), carboxy, ketone, ester, or amide. In some embodiments, R^{2b} (e.g., R²) is deuterium, hydroxyl, halo, alkoxy (e.g., OC₁-C₆ alkyl), alkyl (e.g., C₁-C₆ alkyl), alkenyl (e.g., C₂-C₆ alkenyl), alkynyl (e.g., C₂-C₆ alkynyl), carboxy, ketone, ester, or amide. In some embodiments, R^{2b} is H, deuterium, hydroxyl, halo, alkoxy (e.g., OC₁-C₆ alkyl) or alkyl (e.g., C₁-C₆ alkyl). In some embodiments, R^{2b} is H or alkyl (e.g., C₁-C₆ alkyl). In some embodiments, R^{2b} is H or C₁-C₆ alkyl (e.g., methyl). In some embodiments, R^{2b} is C₁-C₆ alkyl (e.g., methyl).

In some embodiments, R^{3b} is a substituted or unsubstituted carbocyclyl (e.g., C₃-C₁₂ cycloalkyl), substituted or unsubstituted heterocyclyl (e.g., 3- to 12-membered heterocyclyl), substituted or unsubstituted aryl (e.g., C₆-C₁₀ aryl), or substituted or unsubstituted heteroaryl (e.g., 5- to 12-membered heteroaryl). In some embodiments, R^{3b} is a substituted or unsubstituted heterocyclyl (e.g., 3- to 12-membered heterocyclyl), substituted or unsubstituted aryl (e.g., C₆-C₁₀ aryl), or substituted or unsubstituted heteroaryl (e.g., 5- to 12-membered heteroaryl). In some embodiments, R^{3b} is a substituted or unsubstituted heteroaryl (e.g., 5- to 12-membered heteroaryl).

In some embodiments, R^{3b} is a substituted or unsubstituted aryl (e.g., C₆-C₁₀ aryl). In some embodiments, R^{3b} is unsubstituted C₆-C₁₀ aryl or C₆-C₁₀ aryl substituted with deuterium, hydroxyl, halo, alkoxy, alkyl, alkenyl, alkynyl, carboxy, ketone, ester, or amide. In some embodiments, R^{3b} is aryl (e.g., C₆-C₁₀ aryl) substituted with deuterium, hydroxyl, halo, alkoxy (e.g., OC₁-C₆ alkyl), alkyl (e.g., C₁-C₆ alkyl), alkenyl (e.g., C₂-C₆ alkenyl), alkynyl (e.g., C₂-C₆ alkynyl), carboxy, ketone, ester, or amide. In some embodiments, R^{3b} is a substituted or unsubstituted phenyl.

In some embodiments, R^{3b} is a substituted or unsubstituted heterocyclyl (e.g., 3- to 12-membered heterocyclyl). In some embodiments, R^{3b} is unsubstituted 3- to 12-membered heterocyclyl or 3- to 12-membered heterocyclyl substituted with deuterium, hydroxyl, halo, alkoxy (e.g., OC₁-C₆ alkyl), alkyl (e.g., C₁-C₆ alkyl), alkenyl (e.g., C₂-C₆ alkenyl), alkynyl (e.g., C₂-C₆ alkynyl), carboxy, ketone, ester, or amide. In some embodiments, R^{3b} is unsubstituted 3- to 12-membered heterocyclyl.

In some embodiments, R^{3b} is a substituted or unsubstituted heteroaryl (e.g., 5- to 12-membered heteroaryl). In some embodiments, R^{3b} is unsubstituted 5- to 12-membered heteroaryl or 5- to 12-membered heteroaryl substituted with deuterium, hydroxyl, halo, alkoxy (e.g., OC₁-C₆ alkyl), alkyl (e.g., C₁-C₆ alkyl), alkenyl (e.g., C₂-C₆ alkenyl), alkynyl (e.g., C₂-C₆ alkynyl), carboxy, ketone, ester, or amide. In some embodiments, R^{3b} is heteroaryl (e.g., 5- to 12-membered heteroaryl) substituted with deuterium, hydroxyl, halo, alkoxy (e.g., OC₁-C₆ alkyl), alkyl (e.g., C₁-C₆ alkyl), alkenyl (e.g., C₂-C₆ alkenyl), alkynyl (e.g., C₂-C₆ alkynyl), carboxy, ketone, ester, or amide. In some embodiments, R^{3b} is unsubstituted 3- to 12-membered heteroaryl. In some embodiments, R^{3b} is a substituted or unsubstituted indole. In some embodiments, R^{3b} is unsubstituted indole or indole substituted with deuterium, hydroxyl, halo, alkoxy (e.g., OC₁-C₆ alkyl), alkyl (e.g., C₁-C₆ alkyl), alkenyl (e.g., C₂-C₆ alkenyl), alkynyl (e.g., C₂-C₆ alkynyl), carboxy, ketone, ester, or amide. In some embodiments, R^{3b} is unsubstituted indole.

In some embodiments, R^{4b} is H, deuterium, hydroxyl, halo, alkoxy (e.g., OC₁-C₆ alkyl), alkyl (e.g., C₁-C₆ alkyl), alkenyl (e.g., C₂-C₆ alkenyl), alkynyl (e.g., C₂-C₆ alkynyl), carboxy, ketone, ester, or amide. In some embodiments, R^{4b} is H, deuterium, hydroxyl, halo, alkoxy (e.g., OC₁-C₆ alkyl) or alkyl (e.g., C₁-C₆ alkyl). In some embodiments, R^{4b} is H or halo (e.g., chloro). In some embodiments, R^{4b} is halo (e.g., chloro). In some embodiments, R^{4b} is H or chloro.

In some embodiments, R^{5b} (e.g., R³) is H, deuterium, hydroxyl, halo, alkoxy (e.g., OC₁-C₆ alkyl), alkyl (e.g., C₁-C₆ alkyl), alkenyl (e.g., C₂-C₆ alkenyl), alkynyl (e.g., C₂-C₆ alkynyl), carboxy, ketone, ester, or amide. In some embodiments, R^{5b} (e.g., R³) is deuterium, hydroxyl, halo, alkoxy (e.g., OC₁-C₆ alkyl), alkyl (e.g., C₁-C₆ alkyl), alkenyl (e.g., C₂-C₆ alkenyl), alkynyl (e.g., C₂-C₆ alkynyl), carboxy, ketone, ester, or amide. In some embodiments, R^{5b} is H.

The following represent illustrative embodiments of compounds of the disclosure:

| Ex. # | Structure | Name |
|---|---|---|
| MPOL_A_1 | 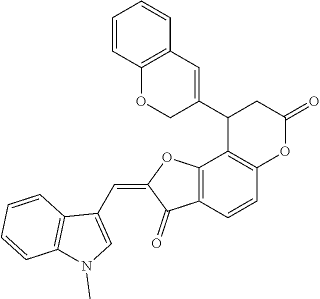 [SMILES: Cn1cc(/C=C2/Oc3c(ccc4c3C(C3=Cc5ccccc5OC3)CC(=O)O4)C2=O)c2ccccc21] | 9-(2H-chromen-3-yl)-2-((1- methyl-1H-indol-3- yl)methylene)-8,9-dihydro-7H- furo[2,3-f]chromene-3,7(2H)- dione |
| MPOL_A_2 | 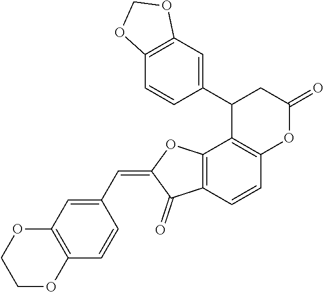 [SMILES: O=C1CC(c2ccc3c(c2)OCO3)c2c(ccc3c2O/C(=C/c2ccc4c(c2)OCCO4)C3=O)O1] | 9-(benzo[d][1,3]dioxol-5-yl)-2- ((2,3- dihydrobenzo[b][1,4]dioxin-6- yl)methylene)-8,9-dihydro-7H- furo[2,3-f]chromene-3,7(2H)- dione |
| MPOL_A_3 | 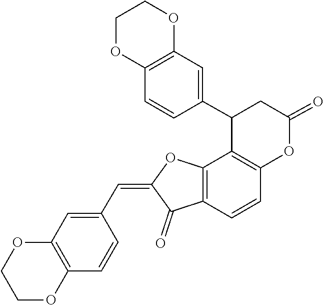 [SMILES: O=C1CC(c2ccc3c(c2)OCCO3)c2c(ccc3c2O/C(=C/c2ccc4c(c2)OCCO4)C3=O)O1] | 9-(2,3- dihydrobenzo[b][1,4]dioxin-6- yl)-2-((2,3- dihydrobenzo[b][1,4]dioxin-6- yl)methylene)-8,9-dihydro-7H- furo[2,3-f]chromene-3,7(2H)- dione |
| MPOL_A_4 | 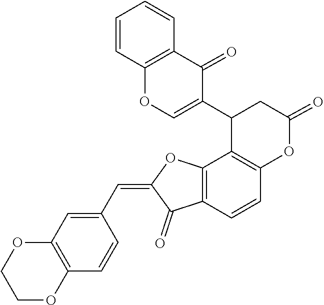 [SMILES: O=C1CC(c2coc3ccccc3c2=O)c2c(ccc3c2O/C(=C/c2ccc4c(c2)OCCO4)C3=O)O1] | 2-((2,3- dihydrobenzo[b][1,4]dioxin-6- yl)methylene)-9-(4-oxo-4H- chromen-3-yl)-8,9-dihydro-7H- furo[2,3-f]chromene-3,7(2H)- dione |
| MPOL_A_5 | 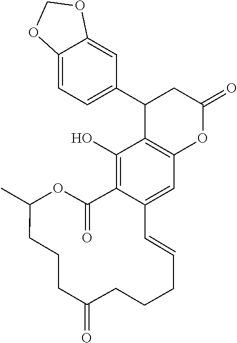 [SMILES: CC1CCCC(=O)CCC/C=C/c2cc3c(c(O)c2C(=O)O1)C(c1ccc2c(c1)OCO2)CC(=O)O3] | 4-(benzo[d][1,3]dioxol-5-yl)-5- hydroxy-8-methyl- 4,8,9,10,11,13,14,15-octahydro- 2H,6H- [1]oxacyclotetradecino[3,4- g]chromene-2,6,12(3H)-trione |
| MPOL_A_6 | 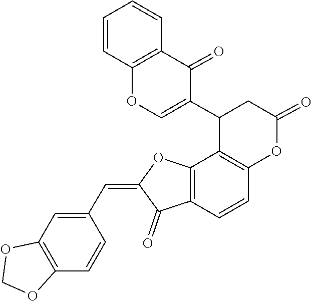 [SMILES: O=C1CC(c2coc3ccccc3c2=O)c2c(ccc3c2O/C(=C/c2ccc4c(c2)OCO4)C3=O)O1] | 2-(benzo[d][1,3]dioxol-5- ylmethylene)-9-(4-oxo-4H- chromen-3-yl)-8,9-dihydro-7H- furo[2,3-f]chromene-3,7(2H)- dione |
| MPOL_A_7 | 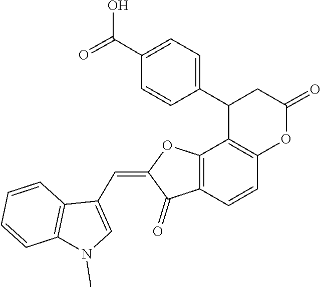 [SMILES: Cn1cc(/C=C2/Oc3c(ccc4c3C(c3ccc(C(=O)O)cc3)CC(=O)O4)C2=O)c2ccccc21] | 4-(2-((1-methyl-1H-indol-3- yl)methylene)-3,7-dioxo- 2,3,8,9-tetrahydro-7H-furo[2,3- f]chromen-9-yl)benzoic acid |
| MPOL_A_8 | 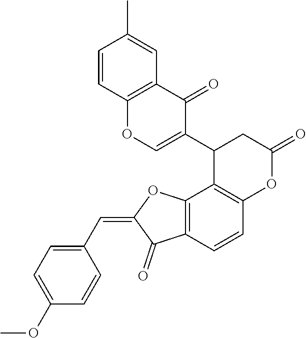 [SMILES: COc1ccc(/C=C2/Oc3c(ccc4c3C(c3coc5ccc(C)cc5c3=O)CC(=O)O4)C2=O)cc1] | 2-(4-methoxybenzylidene)-9- (6-methyl-4-oxo-4H-chromen- 3-yl)-8,9-dihydro-7H-furo[2,3- f]chromene-3,7(2H)-dione |
| MPOL_A_9 | 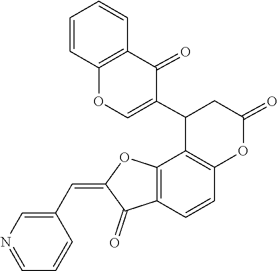 [SMILES: O=C1CC(c2coc3ccccc3c2=O)c2c(ccc3c2O/C(=C/c2cccnc2)C3=O)O1] | 9-(4-oxo-4H-chromen-3-yl)-2- (pyridin-3-ylmethylene)-8,9- dihydro-7H-furo[2,3- f]chromene-3,7(2H)-dione |
| MPOL_A_10 | 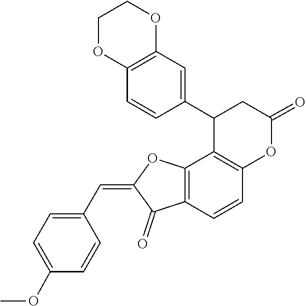 [SMILES: COc1ccc(/C=C2/Oc3c(ccc4c3C(c3ccc5c(c3)OCCO5)CC(=O)O4)C2=O)cc1] | 9-(2,3- dihydrobenzo[b][1,4]dioxin-6- yl)-2-(4-methoxybenzylidene)- 8,9-dihydro-7H-furo[2,3- f]chromene-3,7(2H)-dione |
| MPOL_B_1 | 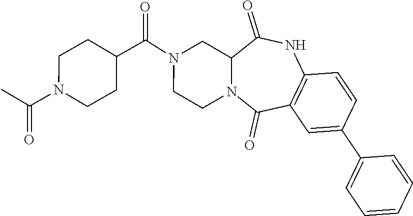 [SMILES: CC(=O)N1CCC(C(=O)N2CCN3C(=O)c4cc(-c5ccccc5)ccc4NC(=O)C3C2)CC1] | 2-(1-acetylpiperidine-4- carbonyl)-8-phenyl-1,3,4,12a- tetrahydrobenzo[e]pyrazino[1,2- a][1,4]diazepine- 6,12(2H,11H)-dione |
| MPOL_C_1 | 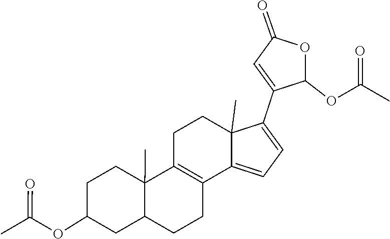 [SMILES: CC(=O)OC1CCC2(C)C3=C(CCC2C1)C1=CC=C(C2=CC(=O)OC2OC(C)=O)C1(C)CC3] | 3-(3-acetoxy-10,13-dimethyl- 2,3,4,5,6,7,10,11,12,13- decahydro-1H- cyclopenta[a]phenanthren-17- yl)-5-oxo-2,5-dihydrofuran-2-yl acetate |
| MPOL_D_1 | 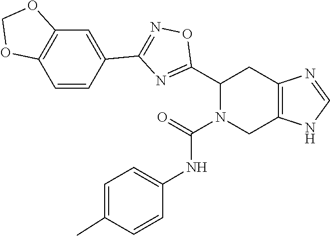 [SMILES: Cc1ccc(NC(=O)N2Cc3[nH]cnc3CC2c2nc(-c3ccc4c(c3)OCO4)no2)cc1] | 6-(3-(benzo[d][1,3]dioxol-5-yl)- 1,2,4-oxadiazol-5-yl)-N-(p- tolyl)-3,4,6,7-tetrahydro-5H- imidazo[4,5-c]pyridine-5- carboxamide |
| MPOL_D_2 | 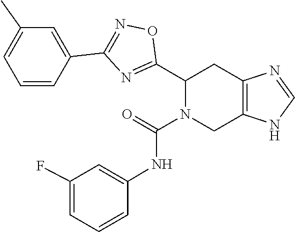 [SMILES: Cc1cccc(-c2noc(C3Cc4nc[nH]c4CN3C(=O)Nc3cccc(F)c3)n2)c1] | N-(3-fluorophenyl)-6-(3-(m- tolyl)-1,2,4-oxadiazol-5-yl)- 3,4,6,7-tetrahydro-5H- imidazo[4,5-c]pyridine-5- carboxamide |
| MPOL_E_1 | 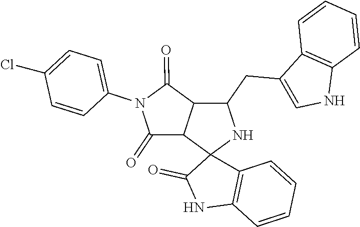 [SMILES: O=C1C2C(Cc3c[nH]c4ccccc34)NC3(C(=O)Nc4ccccc43)C2C(=O)N1c1ccc(Cl)cc1] | 3′-((1H-indol-3-yl)methyl)-5′- (4-chlorophenyl)-2′,3′,3a′,6a′- tetrahydro-4′H-spiro[indoline- 3,1′-pyrrolo[3,4-c]pyrrole]- 2,4′,6′(5′H)-trione |
| MPOL_E_2 | 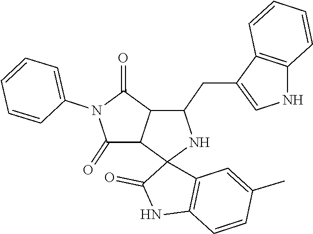 [SMILES: Cc1ccc2c(c1)C1(NC(Cc3c[nH]c4ccccc34)C3C(=O)N(c4ccccc4)C(=O)C31)C(=O)N2] | 3′-((1H-indol-3-yl)methyl)-5- methyl-5′-phenyl-2′,3′,3a′,6a′- tetrahydro-4′H-spiro[indoline- 3,1′-pyrrolo[3,4-c]pyrrole]- 2,4′,6′(5′H)-trione |
| MPOL_F_1 | 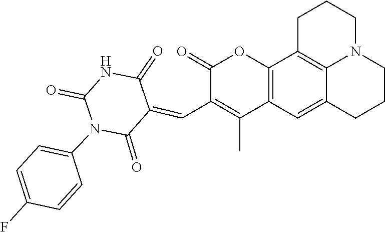 [SMILES: Cc1c(/C=C2\C(=O)NC(=O)N(c3ccc(F)cc3)C2=O)c(=O)oc2c3c4c(cc12)CCCN4CCC3] | 1-(4-fluorophenyl)-5-((9- methyl-11-oxo-2,3,6,7- tetrahydro-1H,5H,11H- pyrano[2,3-f]pyrido[3,2,1- ij]quinolin-10- yl)methylene)pyrimidine- 2,4,6(1H,3H,5H)-trione |
| MPOL_G_1 | 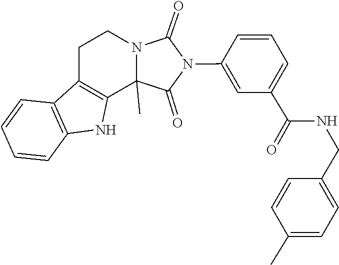 [SMILES: Cc1ccc(CNC(=O)c2cccc(N3C(=O)N4CCc5c([nH]c6ccccc56)C4(C)C3=O)c2)cc1] | 3-(11b-methyl-1,3-dioxo- 5,6,11,11b-tetrahydro-1H- imidazo[1′,5′:1,2]pyrido[3,4- b]indol-2(3H)-yl)-N-(4- methylbenzyl)benzamide |
| MPOL_H_1 | 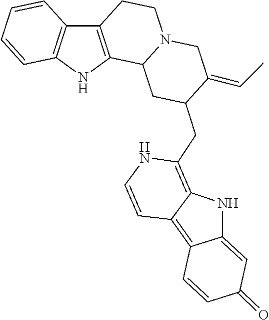 [SMILES: C/C=C1\CN2CCc3c([nH]c4ccccc34)C2CC1Cc1[nH]ccc2c1[nH]c1cc(=O)ccc12] | 1-((3-ethylidene- 1,2,3,4,6,7,12,12b- octahydroindolo[2,3- a]quinolizin-2-yl)methyl)-2,9- dihydro-7H-pyrido[3,4-b]indol- 7-one |

In some embodiments, the disclosure relates to a compound of Formula I, selected from:

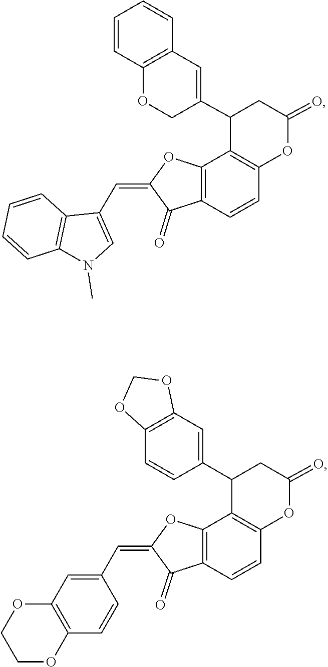 [SMILES: Cn1cc(/C=C2/Oc3c(ccc4c3C(C3=Cc5ccccc5OC3)CC(=O)O4)C2=O)c2ccccc21 ; O=C1CC(c2ccc3c(c2)OCO3)c2c(ccc3c2O/C(=C/c2ccc4c(c2)OCCO4)C3=O)O1]

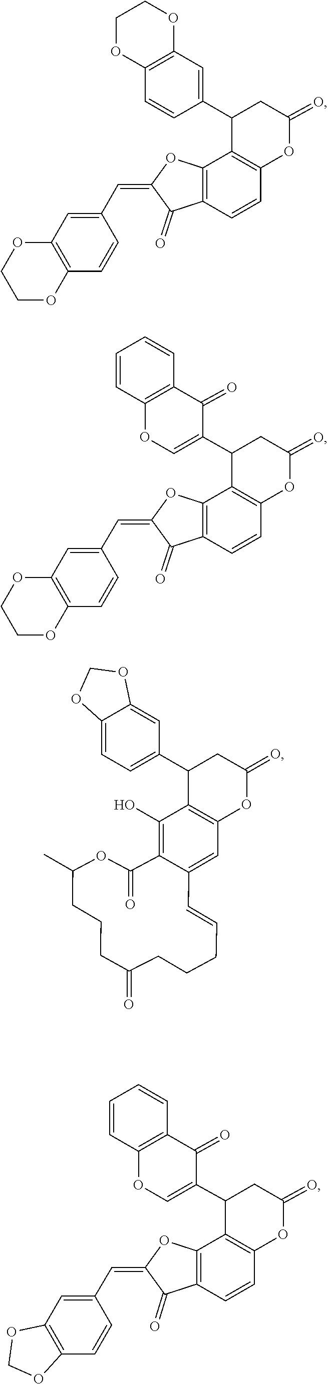 [SMILES: O=C1CC(c2ccc3c(c2)OCCO3)c2c(ccc3c2O/C(=C/c2ccc4c(c2)OCCO4)C3=O)O1 ; O=C1CC(c2coc3ccccc3c2=O)c2c(ccc3c2O/C(=C/c2ccc4c(c2)OCCO4)C3=O)O1 ; CC1CCCC(=O)CCC/C=C/c2cc3c(c(O)c2C(=O)O1)C(c1ccc2c(c1)OCO2)CC(=O)O3 ; O=C1CC(c2coc3ccccc3c2=O)c2c(ccc3c2O/C(=C/c2ccc4c(c2)OCO4)C3=O)O1]

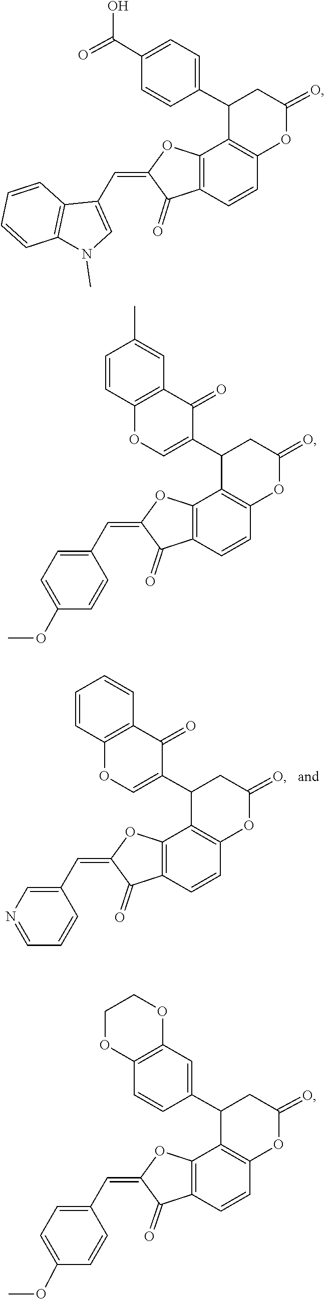

or a pharmaceutically acceptable salt thereof.

In some embodiments, the disclosure relates to a compound of Formula IV, selected from:

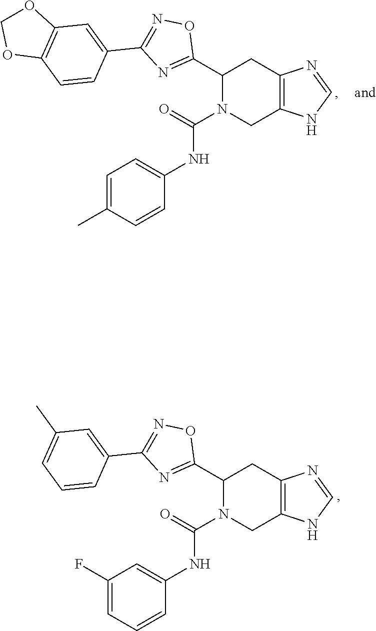 [SMILES: Cc1ccc(NC(=O)N2Cc3[nH]cnc3CC2c2nc(-c3ccc4c(c3)OCO4)no2)cc1 ; Cc1cccc(-c2noc(C3Cc4nc[nH]c4CN3C(=O)Nc3cccc(F)c3)n2)c1]

or a pharmaceutically acceptable salt thereof.

In some embodiments, the disclosure relates to a compound of Formula V, selected from:

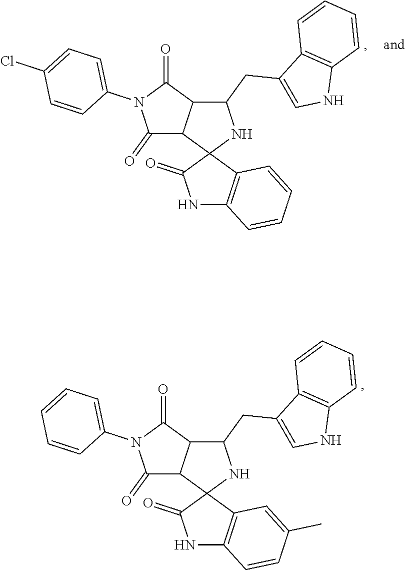 [SMILES: O=C1C2C(Cc3c[nH]c4ccccc34)NC3(C(=O)Nc4ccccc43)C2C(=O)N1c1ccc(Cl)cc1 ; Cc1ccc2c(c1)C1(NC(Cc3c[nH]c4ccccc34)C3C(=O)N(c4ccccc4)C(=O)C31)C(=O)N2]

or a pharmaceutically acceptable salt thereof.

In some embodiments, the disclosure relates to a compound, selected from:

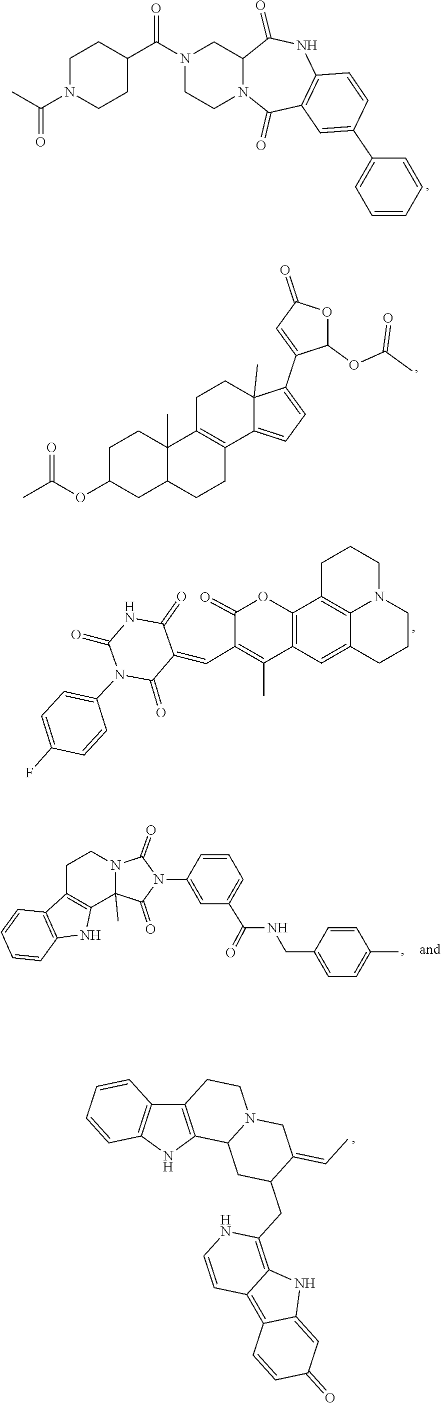 [SMILES: CC(=O)N1CCC(C(=O)N2CCN3C(=O)c4cc(-c5ccccc5)ccc4NC(=O)C3C2)CC1 ; CC(=O)OC1CCC2(C)C3=C(CCC2C1)C1=CC=C(C2=CC(=O)OC2OC(C)=O)C1(C)CC3 ; Cc1c(/C=C2\C(=O)NC(=O)N(c3ccc(F)cc3)C2=O)c(=O)oc2c3c4c(cc12)CCCN4CCC3 ; Cc1ccc(CNC(=O)c2cccc(N3C(=O)N4CCc5c([nH]c6ccccc56)C4(C)C3=O)c2)cc1 ; C/C=C1\CN2CCc3c([nH]c4ccccc34)C2CC1Cc1[nH]ccc2c1[nH]c1cc(=O)ccc12]

or a pharmaceutically acceptable salt thereof.

In some embodiments, the disclosure relates to a compound, selected from:

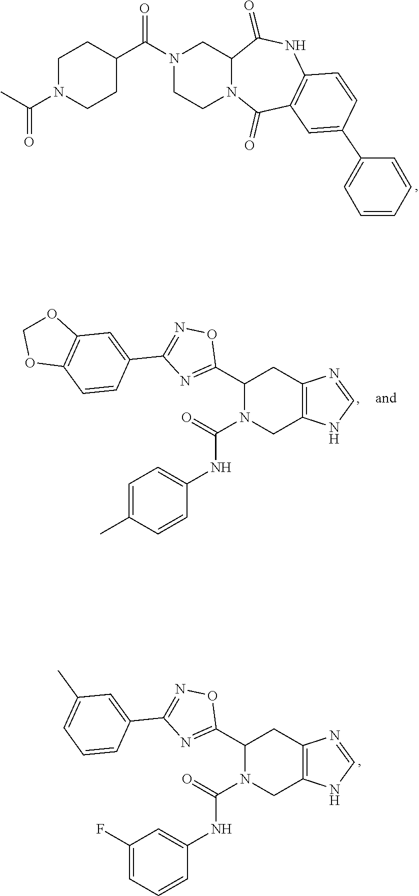 [SMILES: CC(=O)N1CCC(C(=O)N2CCN3C(=O)c4cc(-c5ccccc5)ccc4NC(=O)C3C2)CC1 ; Cc1ccc(NC(=O)N2Cc3[nH]cnc3CC2c2nc(-c3ccc4c(c3)OCO4)no2)cc1 ; Cc1cccc(-c2noc(C3Cc4nc[nH]c4CN3C(=O)Nc3cccc(F)c3)n2)c1]

or a pharmaceutically acceptable salt thereof.

In some embodiments, the disclosure relates to a compound of the structure:

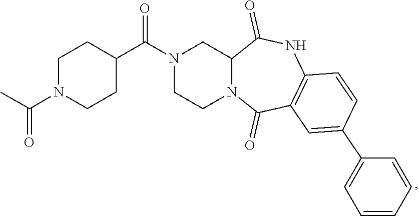 [SMILES: CC(=O)N1CCC(C(=O)N2CCN3C(=O)c4cc(-c5ccccc5)ccc4NC(=O)C3C2)CC1]

or a pharmaceutically acceptable salt thereof.

In some embodiments, the disclosure relates to a method of inhibiting folate uptake into a cell, the method includes a step of administering a compound to a subject (e.g., an animal). In some embodiments, the method further includes a step of inhibiting folate uptake into a cell of a subject (e.g., an animal). In some embodiments, the disclosure relates to a method of inhibiting folate uptake into a cell of an animal, the method includes a step of administering a compound to an animal, and a step of inhibiting folate uptake into a cell of an animal. In some embodiments, the compound inhibits folate uptake (i.e., absorption or endocytosis) into the cell. In some embodiments, the animal is a mouse or a human. In some embodiments, the animal is a human. In some embodiments, the compound inhibits folate uptake via binding to a folate receptor. In some embodiments, the cell is an intestinal cell. In some embodiments, the folate uptake is intestinal folate uptake.

In some embodiments, inhibiting folate uptake prolongs longevity in a subject (e.g., an animal). In some embodiments, inhibiting folate uptake promotes healthy aging of a subject (e.g., an animal).

In some embodiments, the folate is 5-methyl tetrahydrofolate (i.e., L-5-methyltetrahydrofolate (L-5-Me-THF) or L-methylfolate). In some embodiments, the folate is folic acid.

In some embodiments, the folate receptor is a proton-coupled folate transporter (PCFT/SLC46A1), a reduced folate receptor (RFC/SLC19A1), folate receptor alpha, or and folate receptor beta. In some embodiments, the folate receptor is a proton-coupled folate transporter (PCFT/SLC46A1) or a reduced folate receptor (RFC/SLC19A1). In some embodiments, the folate receptor is a proton-coupled folate transporter (PCFT). The folate receptor binds folate to transfer folate inside cells by receptor-mediated endocytosis.

In some embodiments, the compound is administered to an animal at a dose of between about 0.001 to about 1000 mg/kg, of between about 0.01 to about 100 mg/kg, of between about 0.01 to about 50 mg/kg, of between about 0.1 to about 25 mg/kg, of between about 0.1 to about 10 mg/kg, or of between about 0.1 to about 5 mg/kg.

In some embodiments, the inhibition of a folate receptor results in a decrease of folate absorption. In some embodiments, the restriction of folate intake does not adversely affect the health of an animal. In some embodiments, restriction of folate intake improves metabolic activity in an animal. 1C pathways in animals may be uniquely positioned to affect several established hallmarks of aging, such as genomic instability, epigenetic alterations, deregulated nutrient sensing, and mitochondrial dysfunction.

In some embodiments, the inhibition of a folate receptor mimics a diet that is low in folate. In some embodiments, compounds that inhibit folate uptake and absorption as described in the present disclosure could provide a unique intervention to extend healthspan when administered to animals, for example older adults. When used at effective doses, such compounds may moderately reduce folate intake in aged adults, thereby improving healthspan and simultaneously delaying the onset of multiple chronic diseases. One advantage of such compounds over other purported pro-longevity drugs is that they would be much easier to develop as therapeutics because they will work outside the cells.

Furthermore, existing antifolates that kill cells may block cell proliferation by binding to target proteins (e.g., DHFR) inside cells. Thus, compounds that interfere with dietary folate uptake as described in the present disclosure, but do not affect intracellular enzymes that rely on folate, may not be cytotoxic or significantly impair the maintenance of cellular functions.

Various embodiments of the invention are provided throughout the present disclosure. Compounds of the present disclosure include those described in the following numbered embodiments, which are contemplated and non-limiting. Furthermore, methods of using the compounds of the present disclosure include those described in the following numbered embodiments, which are contemplated and non-limiting.

1. A compound of Formula I

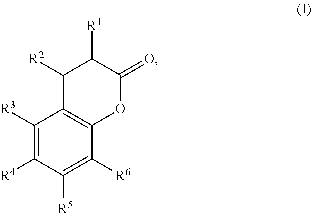

or a pharmaceutically acceptable salt thereof, wherein:
each of R¹ and R⁶ is H;
R² is substituted or unsubstituted carbocyclyl, heterocyclyl, aryl, or heteroaryl; and
each of R³, R⁴, and R⁵ is independently H, deuterium, hydroxy, alkoxy, alkyl, alkenyl, alkynyl, carboxy, ketone, ester, or amide; or two of R³, R⁴, and R⁵ taken together on the carbon to which they are attached form substituted or unsubstituted carbocyclyl, heterocycloalkyl, aryl, or heteroaryl.

2. The compound of clause 1, any other suitable clause, or any combination of suitable clauses, wherein the compound is of Formula Ia

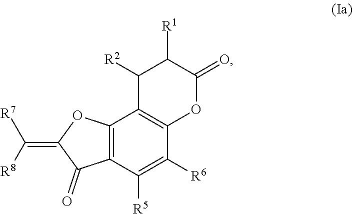

or a pharmaceutically acceptable salt thereof, wherein:
each of R¹, R⁵, and R⁶ is H;
R² is substituted or unsubstituted carbocyclyl, heterocycloalkyl, aryl, heteroaryl; and
each of R⁷ and R⁸ is independently H, or substituted or unsubstituted carbocyclyl, heterocyclyl, aryl, heteroaryl.

3. The compound of clause 1 or clause 2, any other suitable clause, or any combination of suitable clauses, wherein R³ and R⁴ are taken together on the carbon to which they are attached to form

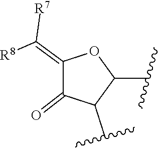

wherein
each of R⁷ and R⁸ is independently H, or substituted or unsubstituted carbocyclyl, heterocyclyl, aryl, heteroaryl.

4. The compound of any one of the preceding clauses, any other suitable clause, or any combination of suitable clauses, wherein R² is substituted or unsubstituted aryl, heterocyclyl, or heteroaryl.

5. The compound of clause 4, any other suitable clause, or any combination of suitable clauses, wherein R² is substituted or unsubstituted C₆-C₁₀ aryl.

6. The compound of clause 4, any other suitable clause, or any combination of suitable clauses, wherein R² is substituted or unsubstituted 3- to 12-membered heterocyclyl.

7. The compound of clause 4, any other suitable clause, or any combination of suitable clauses, wherein R² is substituted or unsubstituted 3- to 12-membered heteroaryl.

8. The compound of clause 4, any other suitable clause, or any combination of suitable clauses, wherein R² is selected from the group consisting of

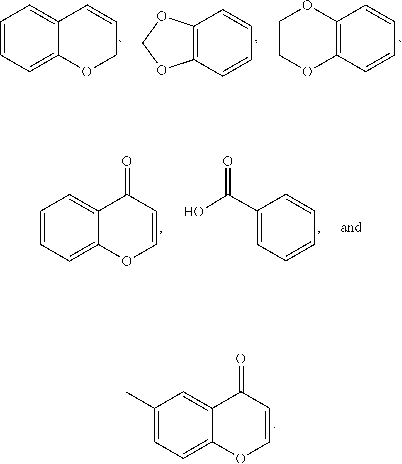

9. The compound of any one of clauses 2 to 8, any other suitable clause, or any combination of suitable clauses, wherein each of R⁷ and R⁸ is independently selected from the group consisting of H,

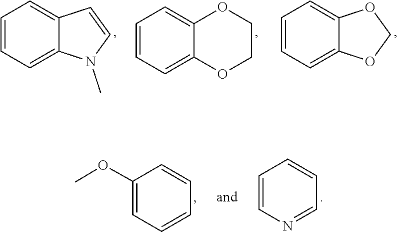

10. The compound of any one of clauses 2 to 9, any other suitable clause, or any combination of suitable clauses, wherein one of R⁷ and R⁸ is H.

11. The compound of clause 1, any other suitable clause, or any combination of suitable clauses, wherein R⁴ and R⁵ are taken together on the carbon to which they are attached form

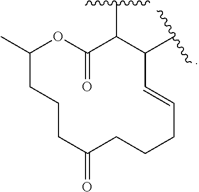 [SMILES: *C1/C=C/CCCC(=O)CCCC(C)OC(=O)C1*]

12. A compound of Formula II

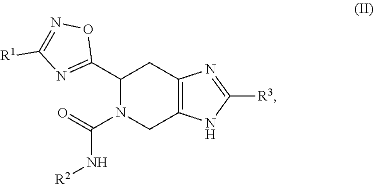

or a pharmaceutically acceptable salt thereof, wherein:
each of R¹ and R² is independently substituted or unsubstituted carbocyclyl, heterocyclyl, aryl, or heteroaryl; and
R³ is H.

13. The compound of clause 12, any other suitable clause, or any combination of suitable clauses, wherein the compound is of Formula IV,

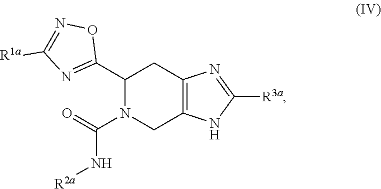

or a pharmaceutically acceptable salt thereof, wherein:
each of R^{1a} and R^{2a} is independently substituted or unsubstituted carbocyclyl, heterocyclyl, aryl, or heteroaryl; and
R^{3a} is H.

14. The compound of clause 12, any other suitable clause, or any combination of suitable clauses, wherein the compound is of Formula IIa

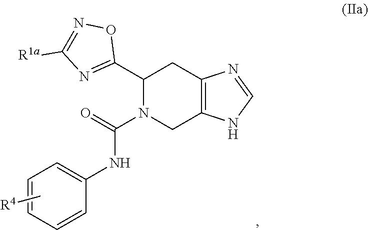

or a pharmaceutically acceptable salt thereof, wherein:
R¹ is substituted or unsubstituted carbocyclyl, heterocyclyl, aryl, or heteroaryl; and
each R⁴ is independently H, deuterium, hydroxyl, halo, alkoxy, alkyl, alkenyl, alkynyl, carboxy, ketone, ester, or amide.

15. The compound of clause 12, any other suitable clause, or any combination of suitable clauses, wherein the compound is of Formula IVa

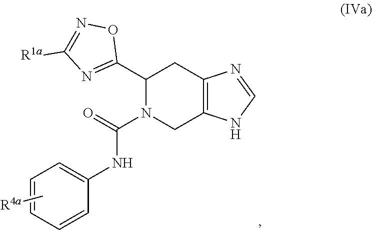

or a pharmaceutically acceptable salt thereof, wherein:
R^{1a} is substituted or unsubstituted carbocyclyl, heterocyclyl, aryl, or heteroaryl; and
R^{4a} is H, deuterium, hydroxyl, halo, alkoxy, alkyl, alkenyl, alkynyl, carboxy, ketone, ester, or amide.

16. The compound of clause 12, any other suitable clause, or any combination of suitable clauses, wherein R¹ (e.g., R^{1a}) is

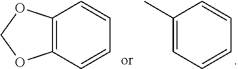

17. The compound of clause 12, any other suitable clause, or any combination of suitable clauses, wherein R² (e.g., R^{2a}) is

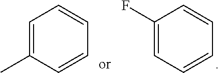

18. A compound of Formula III

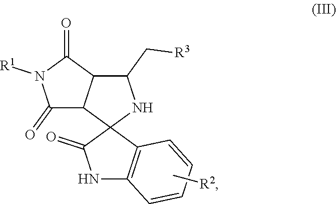

or a pharmaceutically acceptable salt thereof, wherein:
each of R¹ and R³ is independently substituted or unsubstituted carbocyclyl, heterocyclyl, aryl, or heteroaryl; and
each R² is independently H, deuterium, hydroxyl, halo, alkoxy, alkyl, alkenyl, alkynyl, carboxy, ketone, ester, or amide.

19. The compound of clause 18, any other suitable clause, or any combination of suitable clauses, wherein the compound is of Formula V

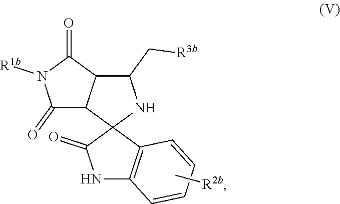

or a pharmaceutically acceptable salt thereof, wherein:
each of R^{1b} and R^{3b} is independently substituted or unsubstituted carbocyclyl, heterocyclyl, aryl, or heteroaryl; and
each R²⁶ is independently H, deuterium, hydroxyl, halo, alkoxy, alkyl, alkenyl, alkynyl, carboxy, ketone, ester, or amide.

20. The compound of clause 18, any other suitable clause, or any combination of suitable clauses, wherein the compound is of Formula IIIa

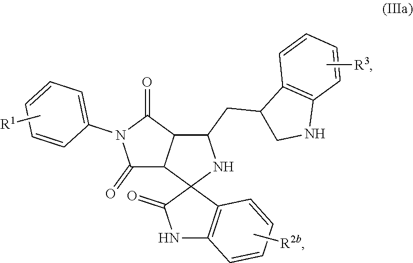

or a pharmaceutically acceptable salt thereof, wherein:
each of R¹, R², and R³ is independently H, deuterium, hydroxyl, halo, alkoxy, alkyl, alkenyl, alkynyl, carboxy, ketone, ester, or amide.

21. The compound of clause 18, any other suitable clause, or any combination of suitable clauses, wherein the compound is of Formula IVa

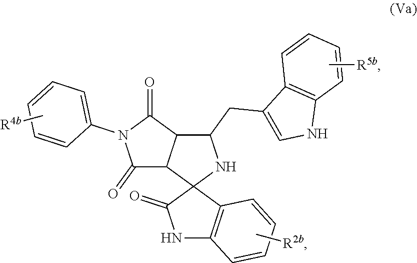

or a pharmaceutically acceptable salt thereof, wherein:
each of R^{2b}, R^{4b}, and R^{5b} is independently H, deuterium, hydroxyl, halo, alkoxy, alkyl, alkenyl, alkynyl, carboxy, ketone, ester, or amide.

22. The compound of any one of the preceding clauses, any other suitable clause, or any combination of suitable clauses, wherein substituted is substituted with one or more of deuterium, hydroxyl, hydroxyalkyl, alkoxy, halogen, alkyl, alkenyl, alkynyl, nitro, silyl, acyl, acyloxy, aryl, cycloalkyl, heterocyclyl, amino, aminoalkyl, cyano, haloalkyl, haloalkoxy, haloalkenyl, haloalkynyl, ketone or oxo, carboxy, amide, ester, OCOCH₂O-alkyl, OP(O)(O-alkyl)₂, or CH₂OP(O)(O-alkyl)₂.

23. A compound selected from:

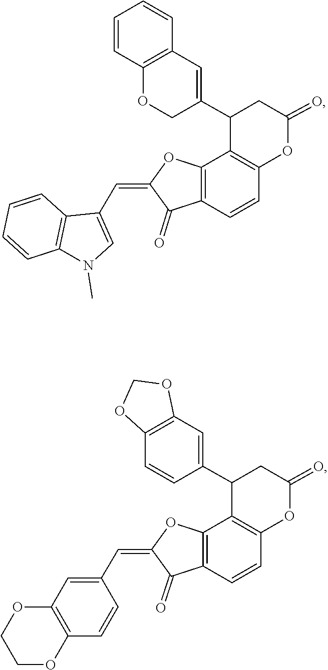 [SMILES: Cn1cc(/C=C2/Oc3c(ccc4c3C(C3=Cc5ccccc5OC3)CC(=O)O4)C2=O)c2ccccc21 ; O=C1CC(c2ccc3c(c2)OCO3)c2c(ccc3c2O/C(=C/c2ccc4c(c2)OCCO4)C3=O)O1]

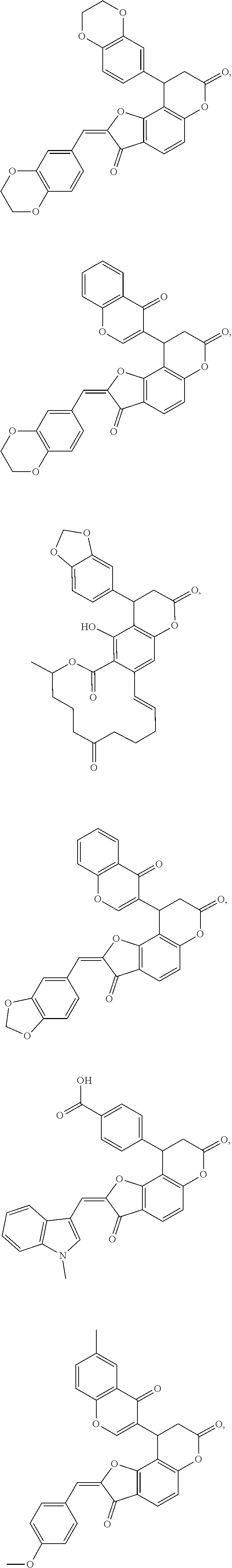 [SMILES: O=C1CC(c2ccc3c(c2)OCCO3)c2c(ccc3c2O/C(=C/c2ccc4c(c2)OCCO4)C3=O)O1 ; O=C1CC(c2coc3ccccc3c2=O)c2c(ccc3c2O/C(=C/c2ccc4c(c2)OCCO4)C3=O)O1 ; CC1CCCC(=O)CCC/C=C/c2cc3c(c(O)c2C(=O)O1)C(c1ccc2c(c1)OCO2)CC(=O)O3 ; O=C1CC(c2coc3ccccc3c2=O)c2c(ccc3c2O/C(=C/c2ccc4c(c2)OCO4)C3=O)O1 ; Cn1cc(/C=C2/Oc3c(ccc4c3C(c3ccc(C(=O)O)cc3)CC(=O)O4)C2=O)c2ccccc21 ; COc1ccc(/C=C2/Oc3c(ccc4c3C(c3coc5ccc(C)cc5c3=O)CC(=O)O4)C2=O)cc1]

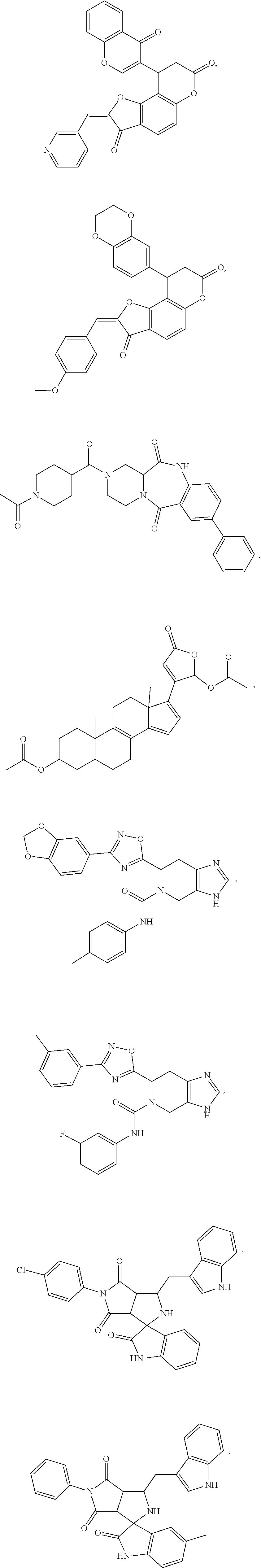

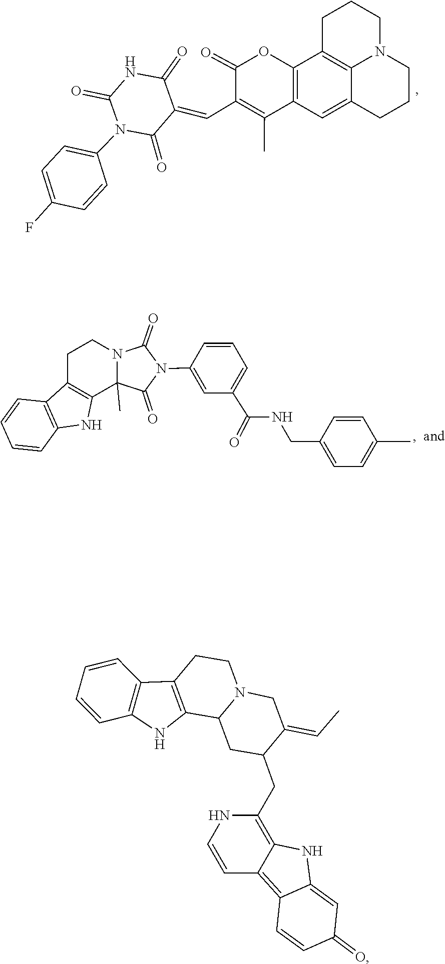 [SMILES: Cc1c(/C=C2\C(=O)NC(=O)N(c3ccc(F)cc3)C2=O)c(=O)oc2c3c4c(cc12)CCCN4CCC3 ; Cc1ccc(CNC(=O)c2cccc(N3C(=O)N4CCc5c([nH]c6ccccc56)C4(C)C3=O)c2)cc1 ; C/C=C1\CN2CCc3c([nH]c4ccccc34)C2CC1Cc1[nH]ccc2c1[nH]c1cc(=O)ccc12]

or a pharmaceutically acceptable salt thereof.

24. The compound of any one of the preceding clauses, any other suitable clause, or any combination of suitable clauses, wherein the compound inhibits a folate receptor.

25. The compound of clause 24, any other suitable clause, or any combination of suitable clauses, wherein the folate receptor is a proton-coupled folate transporter (PCFT).

26. The compound of clause 24, any other suitable clause, or any combination of suitable clauses, wherein the folate receptor is a reduced folate receptor (RFC).

27. A method of inhibiting folate uptake into a cell of a subject, said method comprising the step of administering a compound of any one of clauses 1 to 26 to the subject, wherein the compound inhibits folate uptake into the cell.

28. The method of clause 27, any other suitable clause, or any combination of suitable clauses, wherein the subject is an animal.

29. The method of clause 28, any other suitable clause, or any combination of suitable clauses, wherein the animal is a human.

30 The method of any one of clauses 27 to 29, any other suitable clause, or any combination of suitable clauses, wherein the compound inhibits folate uptake via binding to a folate receptor.

31. The method of clause 30, any other suitable clause, or any combination of suitable clauses, wherein the folate receptor is a proton-coupled folate transporter (PCFT).

32. The method of clause 30, any other suitable clause, or any combination of suitable clauses, wherein the folate receptor is a reduced folate receptor (RFC).

33. The method of any one of clauses 27 to 32, any other suitable clause, or any combination of suitable clauses, wherein the cell is an intestinal cell.

34. The method of clause 33, any other suitable clause, or any combination of suitable clauses, wherein the folate uptake is intestinal folate uptake.

35. The method of any one of clauses 27 to 34, any other suitable clause, or any combination of suitable clauses, wherein the compound is administered to the animal at a dose between about 0.001 to about 1000 mg/kg.

36. The method of any one of clauses 27 to 35, any other suitable clause, or any combination of suitable clauses, wherein the compound is administered to the animal at a dose between about 0.01 to about 100 mg/kg.

37 The method of any one of clauses 27 to 36, any other suitable clause, or any combination of suitable clauses, wherein the compound is administered to the animal at a dose between about 0.1 to about 10 mg/kg.

### EXAMPLES

### Example 1

### Cell Permeability Assays of Compounds

For the instant example, the disclosed compounds can be synthesized and made into water-soluble salts. The compounds can then be tested in permeability assays using Caco-2 monolayer cell cultures [see, e.g., PMID: 17853866]. These cells express high levels of the PCFT/SLC46A1 receptor. The compounds can be tested in triplicate at an initial 10 μM concentration, in the presence of tritiated folic acid at 2 μM, for 2 hours, for their ability to inhibit [3H]-folic acid uptake. The pH of the permeability assays can be 5.5 or 7.5 because transport by the PCFT/SLC46A1 receptor is pH-dependent and maximal at pH 5.5. The inhibitory effect can be compared to inhibition by pemetrexed, generally known to be the most potent known inhibitor of PCFT/SLC46A1. The assays can measure the inhibitory effects of the compounds in folate uptake by cells.

The instant example can be performed using one or more of the exemplary procedures provided in Example 4 as described below.

### Example 2

### Lifespan Analysis of Organisms Exposed to Compounds

The ability of pharmacological or other interventions to extend the lifespan of different organisms is often conserved. For the instant example, the disclosed compounds can be tested for their ability to extend the lifespan of the worm model system Caenorhabditis elegans (N2 strain) [see, e.g., PMID: 19488025]. The organisms can be first exposed to the compounds as adults, 2-3 days after the egg stage until their death, on solid media containing the compounds at the desired concentration. The temperature can be kept constant at 20° C. throughout the assays. The bacteria used as food for the worms (Escherichia coli strain OP-50) can be dead (e.g., killed by lethal doses of ultraviolet radiation) to ensure that any effects on lifespan are not indirect, mediated through bacterial metabolism. These assays can identify the most effective compounds for further testing their effects on healthspan in mice.

The instant example can be performed using one or more of the exemplary procedures provided in Example 4 as described below.

### Example 3

### Healthspan Assays in Mice Exposed to Compounds

Healthspan parameters (e.g., cardiac function, body composition, frailty) can only be measured in live animals in the course of life. For the instant example, mice (strain C57BL/6J) can be evaluated as follows.

Twenty animals for each sex per test treatment can be used for evaluating longevity parameters in mice. From procurement, the mice can be housed to 42 weeks of age, for instance at five animals per cage (females) or singly if necessary (males, to avoid fighting). At approximately 42, 68, 94, and 120 weeks of age, the following assays can be performed: Behavioral (DigiGait treadmill, Novel Object Recognition (NOR), Open Field); Body composition analysis with EchoMRI-100H; Cardiography using the Vevo 3100 Ultrasound device; Blood draw.

At about 45 weeks of age, the animals can be divided into the experimental groups. The compounds can be given orally via milling into the animal chow. Clinical frailty index can be measured at 80 weeks of age and every 4-5 weeks after that. The animals can be euthanized at 120 weeks or when moribund, whichever comes first, followed standard necropsy and tissue/organ collection.

The instant example can be performed using one or more of the exemplary procedures provided in Example 4 as described below.

### Example 4

### Exemplary Procedures

Monitoring behavior-DigiGait Imaging System

The instant protocol evaluates how mice accomplish an increase in their walking speed. It is believed that even small speed changes can have a significant impact on gait indices.
1. Color the bottom of the mouse's feet red with a non-toxic, water-based marker. This aids in the detection of the subject by the camera.
2. Place the subject into the walking compartment and turn the treadmill on speed 1. Suggested speeds are 20, 40, and 60 cm/s.
3. Turn the treadmill on and capture images of the subject moving fluidly and maintaining treadmill speed. Aim to capture ˜4 seconds of video.
4. Note: at the faster speeds, when the animal's stride frequency or cadence is increased, a ˜2-second video is sufficient.
5. After the movie from speed 1 has been archived, increase treadmill speed to speed 2 and repeat.
6. After the movie from speed 2 has been archived, increase treadmill speed to speed 3 and repeat. This approach minimizes the handling of the animal and increases throughput.
7. Actual data collection should take <3 minutes per animal at 3 speeds.
8. Analyze movies via DigiGait. Computation time should take <30 minutes.
9. Return the mouse to clean duplex home cage with food and water after testing.
10. Clean apparatus between trials.
11. Return all mice to the animal facility when testing is complete.

### Monitoring Behavior-Open Field

1. Transport mice to the testing room and let them acclimate for at least 60 minutes.
2. Turn on the recording apparatus.
3. Place the mouse into the center of the open field and allow mice to explore for 20 min.
4. During exploration, record all activity by a video camera mounted above the open field. Scoring behavioral parameters in real-time or following completion of the task using the video recordings. Parameters include:
   a. Distance traveled in first five minutes (a measure of locomotor activity in response to novelty).
   b. Slope of distance traveled (a measure of habituation). The slope is the distance traveled in cm over time spent in the arena. The slope is calculated as the total distance traveled within each of five 4-minute time bins regressed on the bin number. A negative slope indicates habituation to the environment over time.
   c. Percent time spent in corners, periphery and center, and defecation (a reflection of anxiety-like behaviors).
   d. Slopes of percent time spent in corners, periphery, and center (habituation measures and anxiety-like behaviors) over 4-minute bins.
5. Return the mouse to clean duplex home cage with food and water after testing.
6. Clean apparatus between trials.
7. Return all mice to the animal facility when testing is complete.

### Monitoring Behavior-Novel Object Recognition

1. Place the mouse in the empty open field and allow it to explore the open field for 10 min.
2. Return the mouse to its home cage.
3. Clean the open field with a mild detergent containing chlorhexidine acetate (Nolvasan) to minimize olfactory cues.
4. One hour after Step 1, place the two identical objects in the open field, about 15 cm away from the walls. Vary the objects such that each different object will be the novel object in about 50% of the experiments.
5. Place the mouse in the open field, its head positioned opposite the objects. Mice were allowed 10 min of exploration time, which we previously determined as sufficient time where nearly all mice reached at least 45 seconds of exploration. Record the 10 minute familiarization period.
6. Return the mouse to a holding cage where it is temporarily housed separately from the other mice.
7. After the familiarization session, clean the objects with 70% (vol/vol) ethanol and the open field area with Nolvasan detergent to minimize olfactory cues.
8. Dry the objects and the open field before the next use.
9. Replace the two familiar objects, one with a cleaned, familiar object from a different open field arena (to ensure that there are no residual olfactory cues on the previously used object) and the other with a novel object. Place them at the same location, about 15 cm away from the walls.
10. After 15 minutes have passed with the mice in their holding cages, quickly place them back in the open field arena, head positioned opposite the objects.
11. Begin video recording of the arenas, and allow the mice to explore freely for 5 minutes.
12. After the test session, clean the objects with 70% (vol/vol) ethanol and the open field with Nolvasan detergent to minimize olfactory cues.
13. Dry the objects and the open field before the next use.
   Monitoring Cardiac Physiology (with Vevo 3100 Ultrasound)

Heart health can be evaluated using the Vevo 3100 Ultrasound device. Before testing, mice can be anesthetized with isoflurane before using a depilatory agent on their upper ventral side.
1. Place the animal in the induction chamber and anesthetize the mouse with isoflurane at concentrations up to 4% and flow rate of 0.8-2.0 L/min in oxygen. Ensure sedation.
2. Ophthalmic ointment is placed on the eyes to prevent drying of the cornea while the mouse is anesthetized if needed.
3. Once the animal is sedated, move it to a heated EKG plate and nose cone delivering isoflurane at a concentration of 1-2%. Surgical tape is used to position the mouse and place the feet on the EKG plates to monitor hair removal using cream. Only apply the cream to the area of the chest that will be used for imaging. Once the hair is removed, wipe the area with wet gauze to ensure all hair is removed.
4. Apply gel onto the area to be imaged.
5. Lower the probe to the gel until it makes contact, making sure that all probe areas are covered with gel.
6. Image with a VisualSonics VEVO 3100 small animal ultrasound, taken using an ultrasound transmission gel. For aging-related changes in cardiac physiology, the suggested measurements are (From Am J Physiol Heart Circ Physiol 314: H733-H752, 2018):
   Dimensions, FS, volumes, EF, wall thickness
   E and A waves (E/A; transmitral flow)
   E= and A=waves (annular tissue movement)
   To detect more subtle systolic changes, global LV systolic deformation (strain), or peak regional strain rates
7. After the examination, the transmission gel is removed by gently wiping the animal down.
8. After imaging, the mice are returned to their home cage, with a heating pad placed under one side of their cage for 15 minutes while recovery is monitored.

### Body Composition

Body composition can be evaluated using the EchoMRI-100 device. Body weight can be recorded just before the procedure. The procedure involves placing a non-sedated mouse into a tube that is inserted into the machine. Data can be collected in less than one minute, after which the mice are returned to their home cage.

### Blood Collection

Between 0.050 and 0.100 ml of blood can be collected at each bleed using submandibular bleeding. This can be performed by restraining mice with the neck scruff, and a 5 mm lancet can be used to puncture the submandibular vein (located behind the mandible but in front of the ear canal). A swift lancing motion can be used to puncture the vessel. When the sample has been collected, pressure can be immediately applied with a gauze sponge until the bleeding has stopped. Mice can be marked with ear punches.

The samples can be used for the assessment of complete blood counts, differentials, and reticulocytes. Sera can be collected and stored for future analysis of healthspan biomarkers (e.g., IGF1).

### Non-Invasive Frailty Index

The following 31 clinical signs of deterioration were tabulated by Whitehead et al specifically for aging C57BL/6J mice:

| | |
|---|---|
| System and Parameter | Potential Deficits |

Integument

| | |
|---|---|
| Alopecia | Hair loss due to age-related balding and/or barbering (fur trimming). |
| Loss of fur color | Change in fur color from black to gray or brown. |
| Dermatitis | Inflammation, overgrooming, barbering or scratching causing skin |
| | erosion. Can result in open sores anywhere on the body. |
| Loss of whiskers | Loss of vibrissae (whiskers) due to aging and/or whisker trimming. |
| Coat condition | Ruffled fur and/or matted fur. Ungroomed appearance. Coat does not |
| | look smooth, sleek, and shiny. |

Physical/musculoskeletal

| | |
|---|---|
| Tumors | Development of tumors or masses anywhere on the body. |
| Distended abdomen | Enlarged abdomen. May be due to tumor growth, organ enlargement, or |
| | intraperitoneal fluid accumulation. |
| Kyphosis | Exaggerated outward curvature of the lower cervical/thoracic vertebral |
| | column. Hunched back or posture. |
| Tail stiffening | Tail appears stiff, even when the animal is moving in the cage. Tail does |
| | not wrap freely when stroked. |
| Gait disorders | Lack of coordination in movement including hopping, wobbling, or |
| | uncoordinated gait. Wide stance. Circling or weakness. |
| Tremor | Involuntary shaking at rest or during movement. |
| Forelimb grip strength | A decline in forelimb grip strength. |
| Body condition score | Visual signs of muscle wasting or obesity based on the amount of flesh |
| | covering bony protuberances. |

Vestibulocochlear/auditory

| | |
|---|---|
| Vestibular disturbance | Disruption in the ability to perceive motion and gravity. Reflected in |
| | problems with balance, orientation, and acceleration. |
| Hearing loss | Failure to respond to sudden sound (eg, clicker) indicative of hearing loss |
| | or impairment. |

Ocular/nasal

| | |
|---|---|
| Cataracts | Clouding of the lens of the eye. An opaque spot in the center of the eye. |
| Corneal opacity | Development of white spots on the cornea. Cloudy cornea. |
| Eye discharge/swelling | Eyes are swollen or bulging (exopthalmia). They may exhibit abnormal |
| | secretions and/or crusting. |
| Microphthalmia | Eyes are small and/or sunken. May involve one or both eyes. |
| Vision loss | Vision loss, indicated by failure to reach toward the ground when |
| | lowered by the tail. |
| Menace reflex | Rapid eye blink and closure of the palpebral fissure in response to a non |
| | tactile visual threat to the eye. Measures the integrity of the entire visual |
| | pathway including cortical components. |
| Nasal discharge | Signs of abnormal discharge from the nares. |

Digestive/urogenital

| | |
|---|---|
| Malocclusions | Incisor teeth are uneven or overgrown. Top teeth grow back into the roof |
| | of the mouth or bottom teeth are long and easily seen. |
| Rectal prolapse | Protrusion of the rectum just below the tail. |
| Vaginal/uterine/ | Vagina or uterus protrudes through the vagina and vulva. Penis |
| penile prolapse | cannot reenter the penile sheath. |
| Diarrhea | Feces on the walls of the home cage. Bedding adheres to feces in the |
| | cage. Feces, blood, or bedding around the rectum. |

Respiratory

| | |
|---|---|
| Breathing rate/depth | Difficulty breathing (dyspnea), pulmonary congestion (rales), and/or |
| | rapid breathing (tachypnea). |

Discomfort

| | |
|---|---|
| Mouse Grimace Scale | Measure of pain/discomfort based on facial expression. Assessment of |
| | five facial features: orbital tightening, nose bulge, cheek bulge, ear |
| | position (drawn back), or whisker change (either backward or forward). |
| Piloerection | Involuntary bristling of the fur due to sympathetic nervous system |
| | activation. |

Other

| | |
|---|---|
| Temperature | Increase or decrease in body temperature. |
| Weight | Increase or decrease in body weight. |

### Administration of Agents in or on the Animals

In addition to the standard diet (AIN-93G), the mice can be tested on a diet containing the compounds of the present disclosure at the doses determined by the previous experiments in cells and worms, starting late in life, at about 45 weeks old.

### Euthanasia and Standard Diagnostic Necropsy

The animals can be euthanized at 120 weeks of age or when moribund, whichever comes first.

Signs of terminal decline (i.e., moribund) include one or more of the following symptoms:
Lack of response to touch or other stimuli.
Labored breathing or severe respiratory difficulty while at rest, indicated by a regular pattern of deep abdominal excursions or gasping.
Cold to the touch.
Hunched body posture with matted fur.
Signs of sudden weight loss, failure to eat/drink, prominent protrusion of ribs and spine, and sunken hips.
An ulcerated or bleeding tumor, visible to the naked eye and breaking through the skin of the animal, or rapid weight gain associated with visible or palpable masses.
Severe lethargy, as indicated by a reluctance to move when gently prodded.

Euthanasia can be performed by isoflurane at a flow rate or concentration to 5% or greater, continuing isoflurane exposure until one minute after breathing stops. A secondary euthanasia method can be a physical one, by cervical dislocation.

Standard diagnostic necropsies can be performed to examine the appearance of the musculoskeletal structure and evaluate in detail organs for abnormalities.

### Example 5

### Materials and Methods Using Methotrexate (MTX) or ATIC Dimerization Inhibitor

### Replicative Lifespan Assays and Cell Size Measurements in Yeast

The assays were performed in the standard S. cerevisiae strain BY4742 (MATα his3Δ1 leu2Δ0 lys2Δ0 ura3Δ0) on solid rich undefined media (YPD; 1% w/v yeast extract, 2% W/v peptone, 2% W/v dextrose, 2% w/v agar).

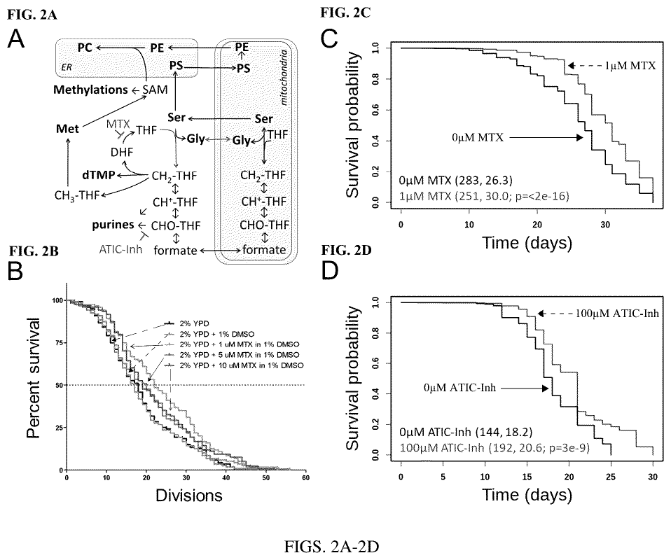

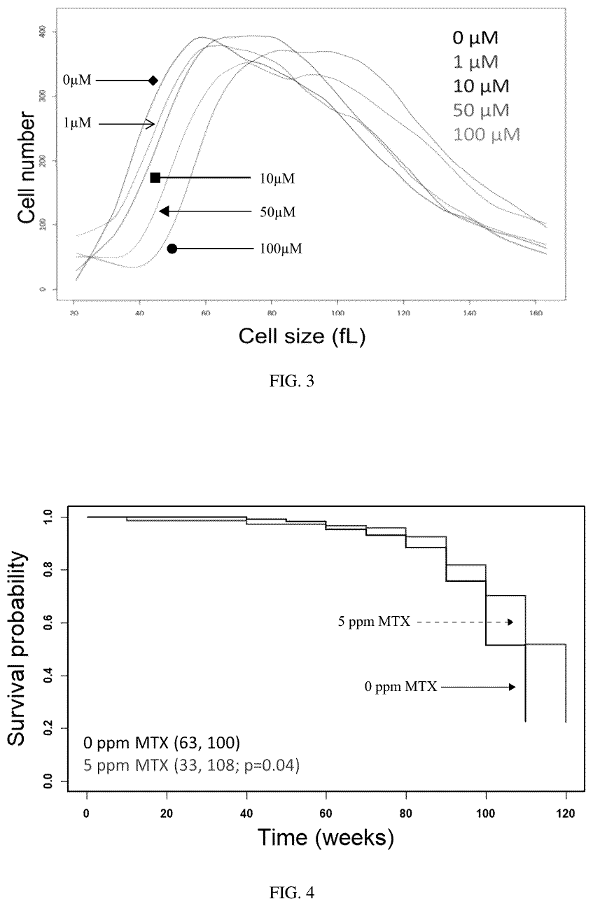

In the instant example, methotrexate (MTX), or the ATIC inhibitor (ATIC-Inh), was dissolved in dimethylsulfoxide (DMSO) and used at the final concentrations shown in FIGS. 2B-2D. The cell size measurements shown in FIG. 3 were also done in strain BY4742 in YPD.

Briefly, overnight cultures were diluted to about 5×10⁵ cells/mL in fresh media, allowed to proliferate for 2-3 h at 30° C., and then methotrexate was added at the indicated final concentrations. 4-5 h later, cell size was measured with a Z2 Channelyzer.

### Lifespan Assays in Worms

The assays were performed at 20° C. using C. elegans strain N2 and a bacterial strain (OP50) commonly used as food for the worms. Briefly, the assays were performed on solid agar nematode growth media (NGM) plates prepared fresh before each experiment. The bacterial lawn was exposed twice to a UV dose of 120 mJ/cm² using a UVC-515 Ultraviolet Multilinker (Ultra-Lum, Inc.). Streaking these UV-exposed bacteria to fresh LB agar plates (1% w/v tryptone, 0.5% w/v yeast extract, 1% w/v sodium chloride) produced no visible colonies. Methotrexate, or the ATIC inhibitor, was first dissolved in DMSO and then added to the media used to prepare the plates after autoclaving (the media were kept in a 50° C. water bath until the plates were poured). Mock-treated control plates contained only DMSO. At the start of each experiment, a sufficient number of eggs were collected from plates without any drugs and then placed on plates containing the indicated doses of each compound tested. After hatching and progression to the adult stage, animals were transferred to new plates (marked as the start of the lifespan assay) containing the drug tested and fluorodeoxyuridine (FUDR; dissolved in water), added at 50 μM to block hatching of new animals. The plates were scored at least every other day until all the worms died. If an animal responded to gentle touch, it was scored as alive, otherwise a death was recorded, and the animal was removed from the plate. Worms were transferred to fresh plates as needed (e.g., if there was evidence of microbial contamination, dryness/cracks on the agar surface, consumption of the bacterial lawn, or hatching of new animals that escaped the FUDR block). The reported lifespans were compiled from several independent experiments done over several months (9-10 months for the methotrexate experiments and 4-5 months for the ATIC inhibitor), each scored by multiple individuals (4-5 persons per experiment). No experiments were excluded from the analysis.

### Mice

Mice were housed in groups of no more than 5 animals per cage at the TAMU Comparative Medicine Program (CMP) facilities. There was no incident of aggressive males needing to be isolated to prevent fighting. All the animals were scored for various phenotypic metrics (see FIG. 2A) at 42 weeks of age before grouping into the different diets. Grouping was randomized based on lean mass values. In addition, partitioning into the two diet groups was balanced for all other metrics evaluated so that for any given mouse in any given group, there were similar mice in the other diet group of the same sex.

The animals were inspected daily and treated for non-life-threatening conditions as directed by the veterinary staff of TAMU-CMP. The only treatment received was for dermatitis (topical solution thrice weekly, as needed). Each room contained sentinel animals housed in filtered cages. When cages were scheduled to be changed, a small amount of dirty bedding was collected from a (rotated) selected rack of cages and added to the fresh sentinel cage to ensure that the sentinel animals were uniformly exposed to any contaminant or pathogen that may be present within the colony. One sentinel animal from each group was sampled quarterly, and the serum was tested for common rodent pathogens. A veterinary pathologist performed microbiology, parasitology, and histology as deemed appropriate.

All animal protocols were approved by the TAMU Animal Care and Use Committee (IACUC 2020-0003; Reference #: 142420). TAMU has NIH/PHS Approved Animal Welfare Assurance (D16-00511 (A3893-01)), and TAMU-CMP is accredited by the Association for the Assessment and Accreditation of Laboratory Animal Care (AAALAC).

### Frailty Index

These measurements indicate age-associated deterioration of health and include the scoring of various integument, physical/musculoskeletal, ocular/nasal, digestive, and respiratory conditions. For example, integument scored alopecia, ruffled/matted coat, and piloerection. Physical/musculoskeletal conditions included tumors, distended abdomen, kyphosis, gait problems, tremors, and body weight. The ocular/nasal category covered cataracts, corneal opacity, eye discharge, and malocclusion. Diarrhea and rectal prolapse were the digestive phenotypes. Respiratory conditions observed were increased breathing rate and labored breathing. A score of 0 was assigned if no sign of frailty was observed and the animal was healthy for that phenotype. Moderate and severe phenotypes were scored as 0.5 and 1, respectively.

### Whole-Body Composition

The analysis was conducted on live, awake animals using a quantitative nuclear magnetic resonance machine (EchoMRI-100; EchoMRI LLC; Houston, TX).

### Gait Measurements

Assays were performed with the DigiGait system (Mouse Specifics, Inc., Framingham, MA). Mouse paw pads were colored red with a non-toxic, washable marker and then placed on the transparent treadmill belt. The belt was started at 0° incline and 24 cm/see speed. Video clips of 3-5 seconds were recorded once the mouse moved freely at full speed. Mice that did not move were retested once and marked as “did not move.” Mice that did not move included a few that were likely physically too frail, but mostly were mice that used the bumper to allow the belt to drag under them without having to move. The video files were imported into the DigiGait Analysis program. After filtering out background noise, analysis graphs were generated and edited with the correct, connect, and exclude functions when necessary.

### Open Field Activity

Total time moving and time in the center of open field arenas were measured using the Noldus Ethovision video tracking software system (version 17.0.1630; Leesburg, VA). Arenas were sectioned into a center, inner zone, outer zone, and a thigmotaxis area along the walls. Mice acclimated to the testing room for at least 15 minutes before being placed in the center of an arena and left undisturbed. For 30 minutes, the camera system tracked their center points.

### Novel Object Recognition

The assays were carried out using a layout consisting of eight gray-colored plexiglass arenas with cameras for video recording. Acquisition of video footage and analysis was done with the Noldus Ethovision software. Objects were pre-tested weeks in advance to ensure that, on average, the mice did not prefer one object over the other. The two types of objects used for the experiment were constructed of plastic Trio blocks, using slightly different blocks and placing a metal spring on one set to make the two different configurations. Objects were thought to be “climb-proof,” but specific mice still climbed on them. Time spent sitting on the objects was removed from the analysis, while quick “climb-throughs” of the objects were kept in the analysis. Eight identical sets of each of the object configurations were constructed.

The testing protocol involved the following steps. Mice in their home cages were placed in the testing room and allowed to acclimate for at least 0.5 h or until their activity slowed and they rested again. Mice (8 total, one in each arena) were then placed in empty testing arenas for a 10-minute habituation phase. Following habituation, mice were placed together back in their home cage for approximately 15 minutes. During that time, arenas were cleaned with a 2% (w/v) chlorhexidine diacetate solution (Nolvasan®), and identical objects were placed in each arena.

To avoid any bias from the order of the objects (familiar vs. novel), half the arenas used one object as the familiar object; half used the other. Mice were then placed back into their same arena, now containing the identical objects, and allowed to explore for 10 minutes, called the familiarization phase. The phase was recorded to ensure that mice explored the objects for at least one minute. At the end of the familiarization, the mice were housed individually in separate holding cages for 15 minutes. During this time, arenas were cleaned with Nolvasan®, and all objects were removed and cleaned with ethanol. New object configurations were set up in each arena, one object being the familiar object seen during the previous phase and one object being the new or novel object. The novel object placement varied between the four arenas to avoid any bias in spatial preference. After 15 minutes in the holding cages, mice were placed back in the same arena for 5 minutes of recording their exploration of the familiar and novel object.

Novel object exploration was analyzed using Noldus Ethovision Analysis software. Each trial was edited to ensure correct recognition of the head vs. tail. Software arena settings were such that equal-sized regions around each object were drawn, and the software calculated the time of the mouse's nose in the area of interest around either the familiar or novel object. The amount of time the nose point was within 2 cm of either object was used to calculate a discrimination index (DI). The DI here was defined as the time spent exploring the novel object minus the time spent exploring the familiar object, which was then divided by total exploration time. A discrimination index greater than or equal to 0.06 was considered a preference for the novel object.

### Echocardiography

Examinations were performed using the FujiFilm VisualSonics Vevo® 3100 high-frequency ultrasound system. Mice were anesthetized using an isoflurane anesthesia chamber (SomnoSuite Mouse Anesthesia System). Once unconscious, mice were placed supine on a heated imaging platform (37° C.) and provided a constant flow of isoflurane for the procedure. Limbs were carefully taped over electrodes containing electrode conductivity gel to maintain body position and monitor heart rate and respiration. Nair™ hair removing lotion was used to remove fur from the thoracic region of the animal. After removing the fur and cleaning with a damp cloth, ultrasound gel was added to the animal's chest for imaging. Mouse heart rates were kept between 285 and 500 bpm for imaging. Analysis was performed with the Vevo LAB software.

### Metabolic Monitoring

PhenoMaster metabolic cages (TSE systems; Chesterfield, MO) were used to evaluate 32 individually housed mice (8 mice per sex per diet group). The system has small mammal gas calorimetry sensors to measure the animal's oxygen consumption and carbon dioxide production to calculate key metabolic parameters, including the respiratory exchange rate (RER). Energy expenditure, food intake, water consumption, body weight, and physical activity were simultaneously recorded over three consecutive days (72 h). Only data for the last 24 h of the experiment were used in the analysis to give the mice two days of acclimation to the new environment.

### Blood Collections

Except for the terminal collection by cardiac puncture, all other blood collections were from the submandibular (facial) vein. The professional staff of TAMU-CMP did all the collections. For the terminal collection, each mouse was euthanized in a carbon dioxide chamber, and a cardiac puncture was performed soon after. Blood was placed in sample tubes with a clotting activator/gel and allowed to clot for at least 20 minutes. Tubes were then spun at 5,000 g for 5 minutes. The clarified serum was aliquoted and stored at −80° C.

### Complete Blood Counts (CBC)

Samples were in Microvette® CB 300 EDTA K2E capillary blood collection tubes. The tubes were inverted 10-15 times again before measuring with the Abaxis VetScan HM5 Color Hematology System.

### Inflammatory Cytokines and Chemokines

Serum samples were sent to Eve Technologies (Calgary, Alberta, Canada) and measured with multiplex laser bead array technology (test MD32). The measurements for each mouse sample were done in duplicate.

### Fecal Microbiome Analysis

There was no microbiome normalization between groups prior to the beginning of the experiment. Mouse fecal pellets were gathered by positioning the mice on a paper towel beneath an overturned glass beaker. A minimum of three fecal pellets from each animal were transferred into cryovials using sterile forceps. The samples were preserved at −80° C. and shipped to Zymo Research, where they were processed and analyzed with the ZymoBIOMICS® Shotgun Metagenomic Sequencing Service (Zymo Research, Irvine, CA). For DNA extraction, the ZymoBIOMICS®-96 MagBead DNA Kit (Zymo Research, Irvine, CA) was used according to the manufacturer's instructions. Genomic DNA samples were profiled with shotgun metagenomic sequencing. Sequencing libraries were prepared with Illumina® DNA Library Prep Kit (Illumina, San Diego, CA) with up to 500 ng DNA input following the manufacturer's protocol using unique dual-index 10 bp barcodes with Nextera® adapters (Illumina, San Diego, CA). All libraries were pooled in equal abundance. The final pool was quantified using qPCR and TapeStation® (Agilent Technologies, Santa Clara, CA). The final library was sequenced on the NovaSeq® (Illumina, San Diego, CA) platform. The ZymoBIOMICS® Microbial Community DNA Standard (Zymo Research, Irvine, CA) was used as a positive control for each library preparation. Negative controls (i.e. blank extraction control, blank library preparation control) were included to assess the level of bioburden carried by the wet-lab process.

Raw sequence reads were trimmed to remove low quality fractions and adapters with Trimmomatic-0.33: quality trimming by sliding window with 6 bp window size and a quality cutoff of 20, and reads with size lower than 70 bp were removed. Antimicrobial resistance and virulence factor gene identification was performed with the DIAMOND sequence aligner. Microbial composition was profiled with Centrifuge using bacterial, viral, fungal, mouse, and human genome datasets. Strain-level abundance information was extracted from the Centrifuge outputs and further analyzed to perform alpha- and beta-diversity analyses and biomarker discovery with LEfSe with default settings (p>0.05 and LDA effect size>2).

### Serum Folate Assays

Liver and serum folate concentrations were measured by the Lactobacillus casei (L. casei) microbiological assay. L. casei growth was quantified at 595 nm on an Epoch Microplate Spectrophotometer (Biotek Instruments). Total folate measurements for the liver samples were normalized to protein concentration as measured using the Lowry-Bensadoun Assay.

### Uracil in Genomic DNA

Liver genomic DNA was isolated using the High Pure PCR Template Preparation Kit (Roche), followed by RNAse A treatment. As previously described, two micrograms of DNA were treated with uracil DNA glycosylase. Samples were derivatized, and uracil levels were quantified using gas chromatography-mass spectrometry.

### Histopathology

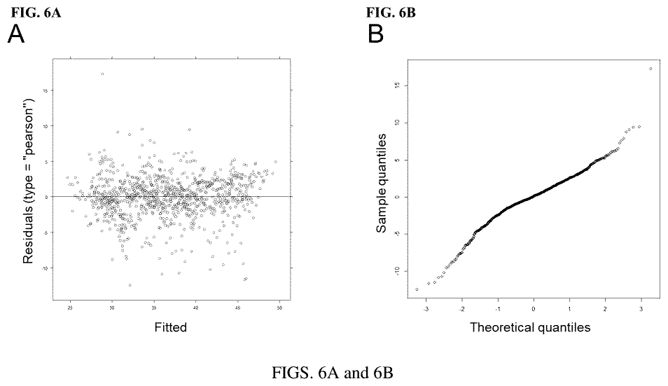

Following euthanasia, ten organs were harvested from each mouse (see FIG. 6—Supplement 1). The samples were then fixed in 10% neutral buffered formalin at room temperature for 48 h and stored in 70% ethanol until processing. Tissue sections were processed using Leica ASP300 Tissue Processor for 4 h before being embedded in paraffin. The paraffin-embedded samples were sectioned at 5 μm, followed by hematoxylin and eosin (H&E) staining, using a Leica HistoCore SPECTRA stainer. In a blinded manner, a board-certified veterinary pathologist (L.G.A.) evaluated histologic sections of all tissues using brightfield microscopy, scoring tissue damage on a scale from 0 to 4.

### Metabolomic Profiling

The untargeted, primary metabolite and biogenic amine analyses were done at the West Coast Metabolomics Center at the University of California at Davis, according to their established mass spectrometry protocols. Extract preparation was also done at the same facility from ˜10 mg of liver tissue in each sample, provided frozen (at −80° C.). To identify significant differences in the comparisons among the different groups, we used the robust bootstrap ANOVA via the t1waybt function of the WRS2 R language package (Mair and Wilcox, 2020). Detected species that could not be assigned to any compound were excluded from the analysis.

### Amino Acid Analysis

Serum samples were used for the PTH-based amino acid done at the Texas A&M Protein Chemistry Facility. Statistical tests for significant differences between the different strains were done as described above for the other metabolites.

### DNAge Analysis

Liver samples (˜15 mg) collected at euthanasia were placed in 0.75 mL of 1×DNA/RNA Shield™ solution (Zymo Research, Irvine, CA), shipped to Zymo Research, and processed with DNAge® Service according to their established protocols. Briefly, after DNA extraction, the EZ DNA Methylation-Lightning Kit (Zymo Research, Irvine, CA) following the standard protocol was used for bisulfite conversion. Samples were enriched specifically for the sequencing of >1000 age-associated gene loci using Simplified Whole-panel Amplification Reaction Method (SWARM®), where specific CpGs are sequenced at minimum 1000× coverage. Sequencing was run on an Illumina NovaSeq instrument. Sequences were identified by Illumina base calling software then aligned to the reference genome using Bismark. Methylation levels for each cytosine were calculated by dividing the number of reads reporting a “C” by the number of reads reporting a “C” or “T”. The percentage of methylation for these specific sequences were used to assess DNA age according to Zymo Research's proprietary DNAge® predictor which had been established using elastic net regression to determine the DNAge®.

RNA Preparation from Liver Tissue

Liver samples were collected at euthanasia and stored at −80° C. in 1×DNA/RNA Shield™ from Zymo Research. For RNA extraction, approximately 15 mg of the stored liver tissue was resuspended in 0.3 mL of fresh DNA/RNA Shield™ and processed using Zymo Research's Quick-RNA™ Miniprep Plus Kit. Extraction was performed according to the manufacturer's protocol with Proteinase K digestion for 4 h at room temperature, followed by a brief centrifugation to remove particulate debris. RNA lysis buffer from the kit was added to the clarified supernatant, and column purification with DNase I treatment was again performed according to the manufacturer's protocol. RNA was eluted in water and stored at −80° C.

### RNAseq

Initial Quality Control was done for library construction to assess RNA concentration, OD ratios, and integrity. mRNA was isolated from 150 ng total RNA using a Nextflex Poly-A Selection kit (Perkin Elmer, Waltham, MA, USA). cDNA libraries were prepared using a Nextflex Rapid Directional RNA 2.0 kit, miniaturized to ⅖ reaction volume, and automated on a Sciclone NGSx liquid handler. The 39 libraries were pooled by equal mass. The pool was sequenced on one Illumina NovaSeq S1 flowcell using the paired-end 2×50 bp recipe, which yielded 1,778 million raw clusters, with an average of 45 million clusters per sample. The sequencing raw data were mapped and quantified using the DRAGEN RNASEq pipeline with default parameters on a DRAGEN Bio-IT Platform with FPGA acceleration. 11% of the bases were trimmed based on quality (minimum quality of 24) and adapters (Stringency of 5). The resulting reads had an average mapping rate of 85% across the samples. The reference genome used was Mouse Build 39 June 2020, GCA_000001635.9. Transcript per million (TPM) values were used in all downstream analyses. All the data have been deposited to the Gene Expression Omnibus (GEO; Accession GSE245438) and are publicly available.

### Immunoblots

In all cases, liver tissue samples were collected at euthanasia. Approximately 20 mg of frozen liver tissue stored in −80° C. was placed into 0.1 mL cold RIPA buffer (150 mM NaCl, 1.0% IGEPAL® CA-630, 0.5% sodium deoxycholate, 0.1% SDS, 50 mM Tris, pH 8.0) containing protease and phosphatase inhibitors. Tissues were manually homogenized with a disposable pestle and microcentrifuge tube. Sample volume was brought to 0.4 mL with additional cold RIPA buffer and the lysates were centrifuged at 12,000 rpm. Clarified lysate was transferred to a new tube and an aliquot removed for immunoblotting.

For the RPS6 immunoblots, two gels were loaded for each set of samples, one to detect total RPS6, the other to be used for detection of phosphorylated RPS6. RPS6 phosphorylation was detected by a specific rabbit monoclonal antibody against phosphorylated Ser235/236 of the human RPS6 protein, followed by an HRP-conjugated anti-rabbit secondary antibody. Total amounts of RPS6 were detected with a rabbit anti-RPS6 polyclonal antibody. The phosphorylated RPS6 signal from each mouse sample was divided by the total RPS6 signal in that sample. To account for spurious differences arising among the different gels, on each gel we run samples from the same sex, but from the two diet groups. Then, each P-RPS6: RPS6 ratio was divided by the average of these ratios from all the samples on that gel (i.e., from both diet groups), and these are the values used to generate FIG. 21.

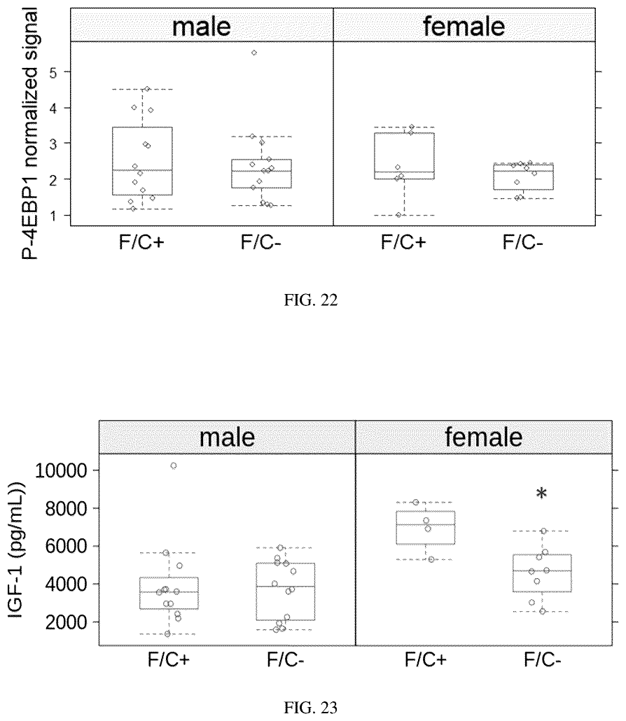

For the 4EBP1 immunoblots, the samples were prepared and run on SDS-PAGE gels as above. It was verified that the signal from the phospho-specific antibody was sensitive to treatment of λ-phosphatase (not shown). The quantification of the signals is shown in FIG. 22.

### ELISAs

Commercially available kits were used to measure serum levels of IGF-1 according to the manufacturer's instructions. Measurements were taken using a Tecan SPARK instrument.

### Statistical Analysis

Data were analyzed using the latest version of the R language. The corresponding R packages and tests are described in the text in each case. Briefly, longitudinal measurements were evaluated with mixed effects models using the Ime4 and lmer packages. A typical function had the following syntax: lmer(′measurements'˜diet+time+sex+(1|ID), data=‘dataset’). It fits a model where ‘measurements’ is the response variable, and ‘diet’, ‘time’, and ‘sex’ are the fixed effects. ‘ID’ is an individual mouse, considered as a random effect to account for the non-independence of measurements within the same subject. This was a typical setup for a longitudinal evaluation where multiple measurements were taken from the same subjects over time. For survival analyses, the survival package was used. For group comparisons of non-longitudinal data, the non-parametric Wilcoxon rank-sum test was used, implemented with the base “wilcox.test” R function, or the robust bootstrap ANOVA, implemented with the “t1waybt” function of the WRS2 package. All the replicates in every experiment shown were biological ones. The number of biological replicates analyzed in each case is indicated in the text and the corresponding figures. No data or outliers were excluded from any analysis.

### Example 6

### Analysis Using Methotrexate (MTX) or ATIC Dimerization Inhibitor

The materials and methods according to Example 5 were followed for the instant Example.

### Inhibitors of 1C Metabolic Enzymes Extend the Lifespan of Yeast and Worms

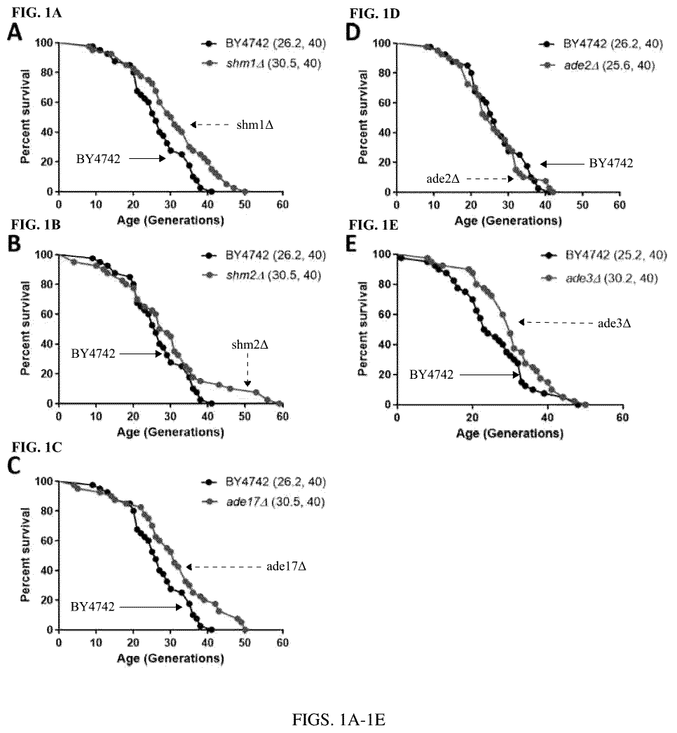

As shown in FIGS. 1A-1E, the deletion of enzymes of one-carbon metabolic pathways extends replicative lifespan in yeast. Loss of SHM2, ADE17, or SHM1 (encoding the mitochondrial serine hydroxymethyltransferase) significantly extended the lifespan of otherwise wild type cells.

Methotrexate (at 0.5-10 μM) increased yeast replicative lifespan (FIG. 2B, p<0.05 based on the log-rank test). The dose with the maximal (˜15%) lifespan extension was 1 μM (FIG. 2B). At such low doses, methotrexate did not affect cell proliferation significantly (not shown). Cell size as a proxy of cell cycle effects was also examined (FIG. 3). Folate deficiencies and drugs that interfere with DNA replication are often associated with increased cell size, leading to megaloblastosis. Because methotrexate inhibits dihydrofolate reductase (DHFR), it limits the levels of THF for thymidylate synthesis and DNA replication. At concentrations up to 10 μM, methotrexate increased cell size slightly (from 47 fL to 52 fL; FIG. 3). At higher concentrations, methotrexate did not extend replicative longevity (FIG. 2B) and increases cell size more significantly (from 47 fL to 56-64 fL, for 50-100 μM, respectively; FIG. 3), indicating a stronger cell cycle block. These data are consistent with the notion that moderate, but not severe, cell cycle delays are associated with longer replicative lifespan in yeast.

Given the conservation of 1C pathways, it was hypothesized that methotrexate may increase the lifespan of wild-type C. elegans animals. Previous studies have shown that treating worms with high levels of methotrexate (220 μM) at the adult stage did not extend their lifespan. Because adult worms are post-mitotic, C. elegans was exposed to the drug continuously, from the embryo stage until death (FIG. 2C). Worms exposed to low doses of methotrexate (1-3 μM) had a longer lifespan (˜15% lifespan extension, p<2E-14 based on the log-rank test, FIG. 2C). At higher doses (10-100 μM), methotrexate did not extend lifespan (not shown), in agreement with previous studies treating adult animals with 220 μM methotrexate. It is also noted that the bacteria used to feed the worms in the instant experiments were killed by ultraviolet radiation to exclude any impacts from bacterial folate metabolism, which is known to affect worm lifespan. Without being bound by any theory, these data may suggest that the longevity extension by methotrexate is conserved between yeast and invertebrates.

To further test the generality of the idea that 1C interventions promote longevity, another commercially available inhibitor of a 1C enzyme was examined. A dipeptide that blocks the requisite dimerization of ATIC (AICAR Transformylase/Inosine Monophosphate Cyclohydrolase; see FIG. 1A) has been previously shown to activate the AMPK signaling pathway and ameliorate the metabolic syndrome in mice. The ATIC inhibitor also increased the lifespan of worms at 100 μM (FIG. 2D). Without inferring any equivalence in outcomes across species, it is noted that this dose is similar to the one used in mice with metabolic syndrome.

Without being bound by any theory, the above data suggest that pharmacologic interventions of 1C metabolism may increase the lifespan of invertebrate model organisms, raising the exciting possibility that 1C interventions may improve longevity in mammals.

It was reasoned that long-term studies might offer helpful information about the role of folate limitation in longevity in mice. Survival curves were generated from available historical data (0-10 ppm MTX) given in alternating weeks in the diet in male and female Swiss mice from 7 to 120 weeks of age. In 5 of the 8 conditions tested, the mean lifespan was longer than in the control group. In one case, it was significantly so (p=0.04, based on the log-rank test, FIG. 4). The number of females used was too low to detect significant differences in lifespan and no healthspan parameters were evaluated in those studies. These limitations notwithstanding, the results are significant because the drug was administered from a young age (7 weeks) when side effects are expected to be more pronounced. The LD50 for methotrexate given to 5-week-old mice is 59 mg/kg, whereas that for 16-week-old mice is 284 mg/kg. Based on these observations, and the results in yeast and worms (FIGS. 2A-2D), the next step was to measure if folate limitation later in life improves healthspan in mice.

Mice Placed Under Dietary Folate Restriction Late in Life Maintain or Increase their Weight and do not Develop Anemia

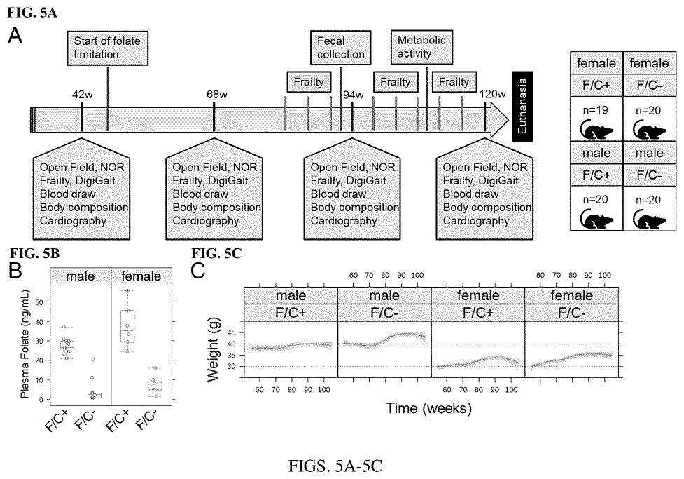

Since relatively little is known about healthspan as a function of dietary folate intake, a study in the long-lived inbred strain C57BL/6J was designed (FIG. 5A). Longevity was not the measured outcome; the mice were sacrificed at 120 weeks. However, with a sample size of 20 mice per group, there was sufficient power to detect significant (p<0.05 and 80% power) differences (1SD) in healthspan parameters. Starting at 52 weeks of age, half the mice were maintained on the standard diet (AIN-93M), and the other half were placed on a folate/choline-deficient (F/C−) diet. Before the diet changes at 52 weeks of age, the mice were randomly assigned to the different test groups based on their lean mass. The standard diet contains 2 mg/kg of folic acid and 2.5 g/kg choline bitartrate (F/C+), while the F/C− diet contains 0 mg/kg of folic acid and 0 mg/kg choline bitartrate. As expected, serum folate levels were greatly reduced in the F/C-groups (p=0.00216 for the females and p=0.00012 for the males, based on the Wilcoxon rank sum test; FIG. 5B).

It was found that the weight of mice from either sex was not reduced from 52 weeks of age when placed on the F/C− diet until the end of the study at 120 weeks of age (FIG. 5C). Instead, male mice on the F/C− diet gained weight (FIG. 5C, compare the two left panels). To evaluate the statistical significance of this observation, a mixed effects regression model was applied to analyze the repeated longitudinal weight measurements using the lme4 and lmer R language packages. The model was valid because from a scatter plot of the standardized residuals vs. the fitted values, the residuals were symmetrically distributed around zero, with approximately constant variance (FIG. 6A). Furthermore, the normality of the residuals was checked with a quantile-quantile plot (FIG. 6B). Based on the mixed effects regression model, there was a negative effect between diet (F/C+) and weight (slope=−1.96, p=0.0313). As expected, there was a small but significant positive relation between weight and time (slope=0.075, p<2E-16) and a strong one between weight and male sex (slope=7.72, p=6.48E-13).

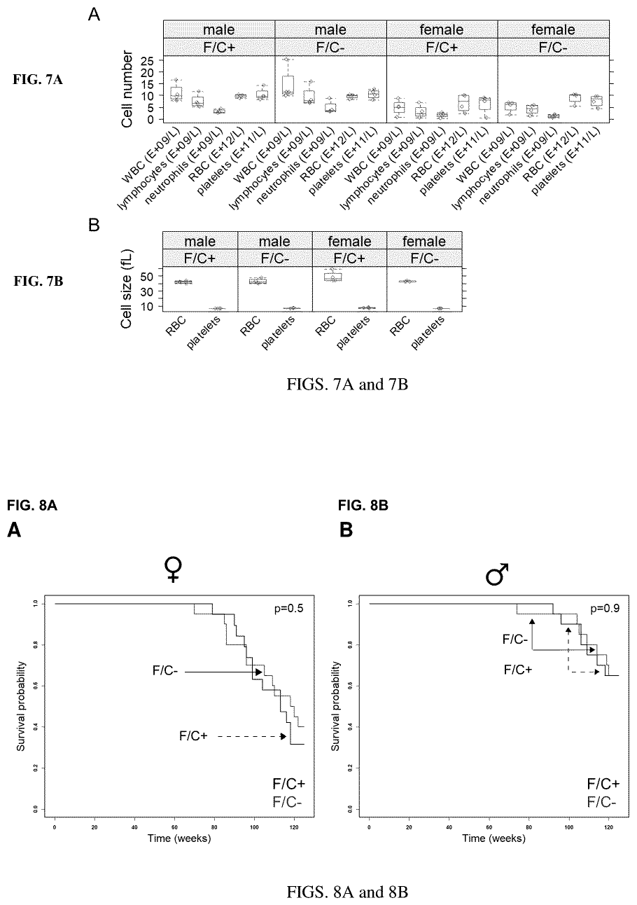

The mice on the F/C− diet were not anemic (FIGS. 7A and 7B). They had the same blood cell counts as the mice on the F/C+ diet (FIG. 7A). There were also no cell size changes or evidence of megaloblastosis (FIG. 7B), which one might expect if DNA replication in erythrocytes was significantly inhibited. Lastly, mice on the F/C− diet did not have reduced survival compared to animals of the same sex that were kept on the F/C+ diet (FIGS. 8A and 8B). It is unclear why the female animals had reduced survival compared to males. Still, the increased mortality of C57BL/6J females is in line with data from the Aged Rodent Colonies maintained by the National Institute on Aging. Females experience 50-66% mortality between 20 and 30 months of age vs. 30-36% mortality for male mice. These data show that dietary folate limitation late in life does not lead to anemia, reduced viability, or reduced body weight. In contrast, at least in the case of males, folate-limited animals have a higher body weight.

### No Adverse Healthspan Metrics in Mice Placed on Dietary Folate Restriction Late in Life

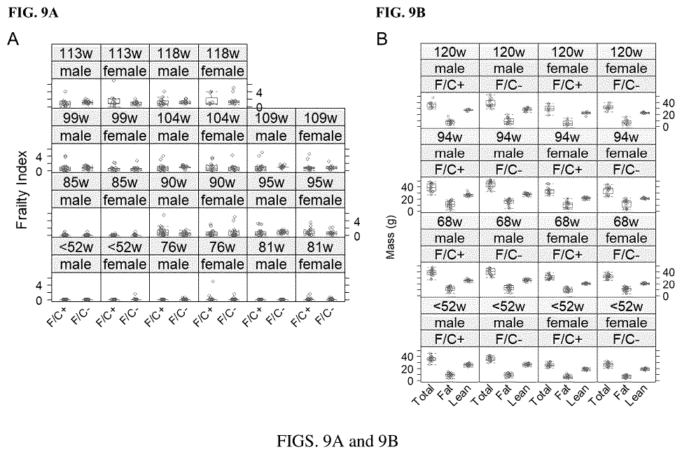

At the indicated times shown in FIG. 5A, various metrics associated with healthspan were evaluated. Mice on the F/C− diet had similar Frailty Index scores (p=0.434, based on a mixed effects regression model) with their counterparts on the F/C+ diet (FIG. 9A). The Frailty Index is a clinically validated metric comprising several visible clinical signs of physical deterioration. Note that at the “<52w” timepoint, the diet had not been switched yet, and all the mice were on the F/C+ diet. However, to accurately track individual animals and visualize the data, the mice that were placed in the different diet groups at 52 weeks of age are also depicted in the same groups at the “<52w” time point.

In addition to the regular body weight measurements described in FIGS. 5A-5C, we also evaluated body composition based on measurements with EchoMRI (FIG. 9B). There was again a significant negative effect from the F/C+ diet on total body mass (slope=−2.1674, p=0.04580, based on a mixed effects regression model). There was also a negative trend between the F/C+ diet and fat mass (slope=−1.6989, p=0.0635), and a weaker negative association with lean mass (slope=−0.5034, p=0.1474), although in the latter cases the effects did not reach the p<0.05 threshold.

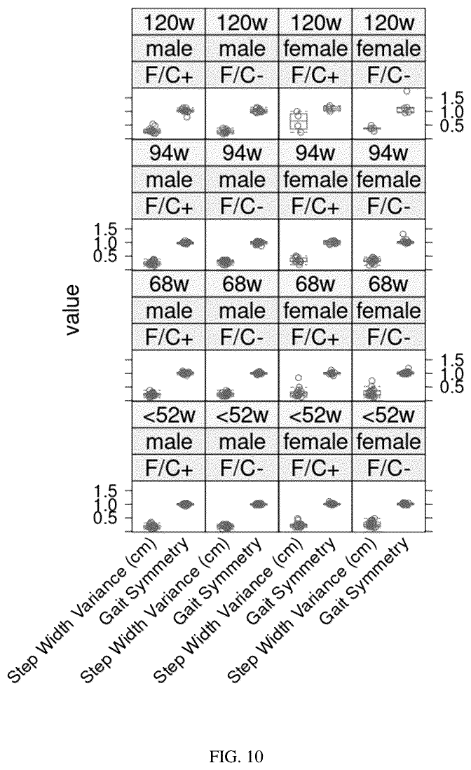

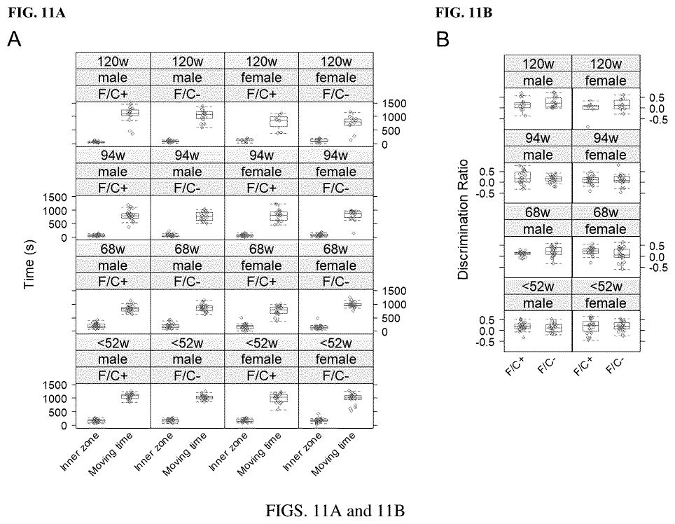

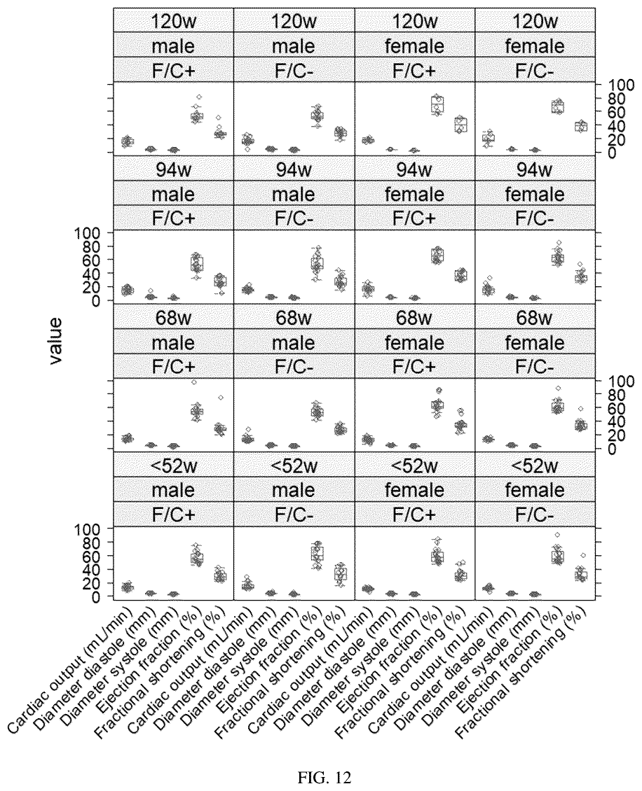

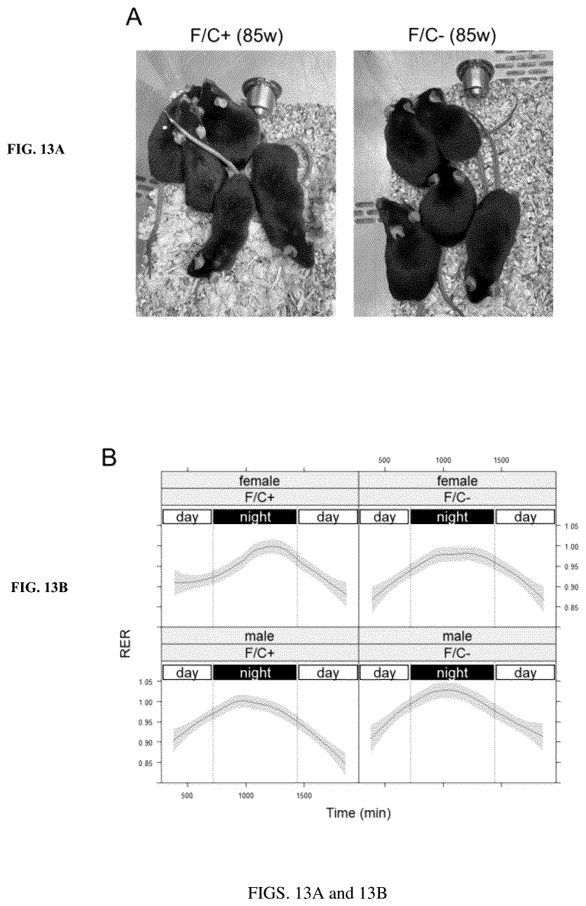

Regarding the other healthspan-related metrics evaluated at 68, 94, and 120 weeks of age (see FIG. 5A), there were no significant diet effects based on mixed effects models. The metrics measured included gait analysis during voluntary walking, which evaluates a wide range of ambulatory problems, using the accurate and high throughput DigiGait system (FIG. 10), open field (FIG. 11A), and novel object recognition (NOR) parameters (FIG. 11B), which evaluate cognitive behavior and memory. In the case of NOR, in addition to the discrimination index (DI) values shown in FIG. 11B, differences in a binary, pass-fail format were also evaluated. A DI>0.06 was chosen as the cutoff for a passing novel object test, as this also correlated visually with what was seen on a heat map of exploration around each object. Based on χ2 tests, there were again no significant diet effects. Lastly, cardiac function was evaluated with echocardiography using the Vevo 3100 Ultrasound device, looking specifically for aging-related changes in cardiac physiology. Cardiac output, systole and diastole diameter, ejection fraction, and fractional shortening were all unaffected by diet (FIG. 12).

### Metabolic Activity of Aged Mice on the Folate-Restricted Diet

Not only were no healthspan metrics adversely affected by limiting dietary folate intake late in life (FIGS. 9A and 9B), but some positive outcomes were observed. At ˜85 weeks of age, the male animals on the folate-limited diet were visibly less gray than their counterparts on the folate-replete diet (FIG. 13A). Although graying is not necessarily associated with declining health, it is usually an age-dependent trait. Notably, recent work in human cells showed that inhibiting the target of rapamycin (TOR) inhibits graying, consistent with the idea that delayed hair graying may be an outcome of interventions that improve healthspan.

Since folate-based 1C chemical reactions are a metabolic hub (see FIG. 2A), individual animals were placed in TSE Phenomaster metabolic cages. Several physiological parameters were measured reporting global metabolic features (FIG. 13B). A metric of particular significance is the respiratory exchange ratio (RER), which is the ratio between the metabolic production of carbon dioxide (CO₂) and the uptake of oxygen (O₂). RER is an indicator of whether carbohydrate or fat is being metabolized. An RER <1 suggests that fat is predominantly used (e.g., during sleep).

A value of 1 is indicative of carbohydrate use. RER can exceed 1 during exercise. How fast the RER changes between periods of inactivity vs. activity (e.g., during diurnal transitions) reflects metabolic plasticity, which is expected to decline with age. Male animals on the F/C+ diet increased their RER as they became more active during the night (FIG. 13B, left bottom panel). The rise in the RER was slower in the female animals on the F/C+ diet (FIG. 13B, left top panel), suggesting reduced metabolic plasticity. Their counterparts on the F/C− diet maintained metabolic plasticity, transitioning much faster to carbohydrate-based fuel consumption (FIG. 13B, compare the two top panels). Furthermore, the male mice on the F/C− diet had higher RER values at night than those on the F/C+ diet (FIG. 13B, compare the two bottom panels). Based on mixed effects regression models for the period during the transition from daytime to night (time 700-1100 min in FIG. 4B), the effects of time and sex (males) on the RER increase were positive and significant (p=1E-06 and p=0.0101, respectively), while there was a negative association with the F/C+ diet (p=0.0806). Without being bound by any theory, these data suggest that a late-life folate-limited diet might have metabolic benefits, albeit for different reasons in the two sexes. Female mice had improved metabolic plasticity, while males reached higher RER values.

### Changes in the Intestinal Microbiome of Mice Limited for Folate Late in Life

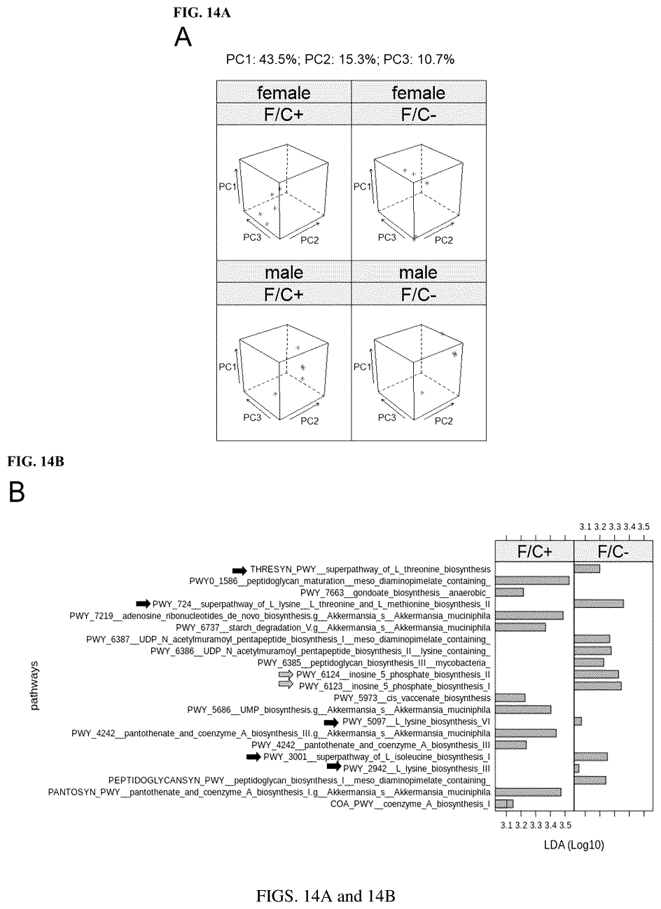

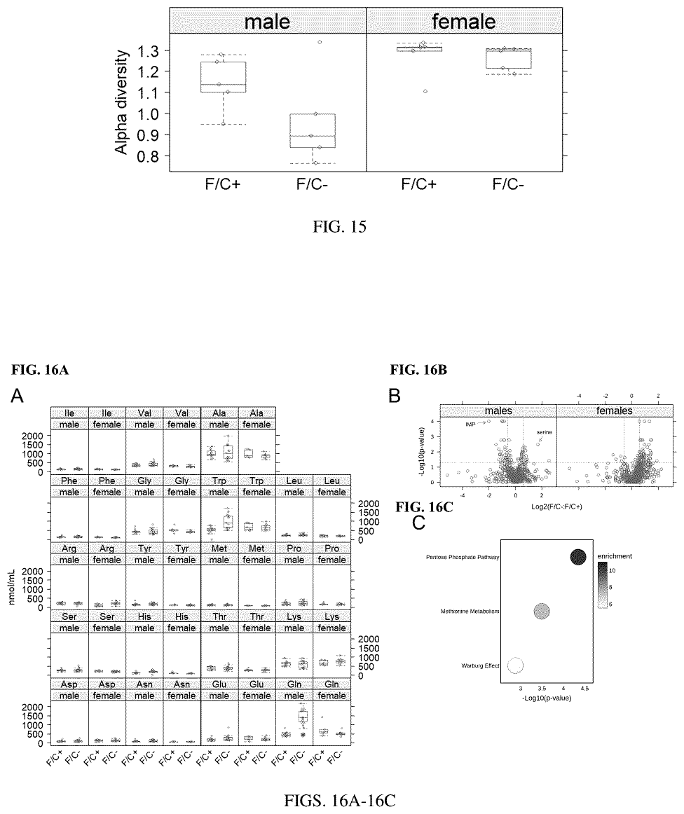

Since the microbiome may contribute to 1C metabolism, the DNA of the fecal microbiome at 90 weeks of age were sampled and sequenced. The analysis yielded standard diversity metrics to assess differences in the fecal microbiome's makeup among the mice groups (FIGS. 14A and 14B). The different sex and diet groups had an easily distinguishable gut microbiome, occupying different areas of principal component analysis graphs (FIG. 14A), based on Bray-Curtis β-diversity dissimilarity indices. The intestinal microbiome of male mice on the F/C− diet was not less diverse (p=0.222, based on the Wilcoxon rank sum test; FIG. 15).

Biomarkers from the same metagenomic datasets were examined to place the above differences in the context of 1C metabolism. The LEfSe computational pipeline was used to determine the features most likely to explain the observed differences. Notably, the LEfSe algorithm incorporates effect sizes, ranking the relevance of the identified biomarkers and enabling their visualization through the computed linear discriminant analysis (LDA) scores. The microbiome pathway changes were essentially dimorphic for sex. Only an enrichment for Coenzyme A biosynthesis was evident in male and female mice on the folate-replete diet (FIG. 14B, bottom bar). All other changes were from one sex (to simplify the visualization, the data from both sexes were grouped). Overall, pathways involved in amino acid (FIG. 14B; black arrows) and IMP (FIG. 14B; gray arrows) synthesis were enriched in mice on the folate-limited dict. Without being bound by any theory, these data suggest that under dietary folate limitation, the microbiome may be a source of metabolites that are known outputs of 1C metabolism (e.g., methionine, and IMP, from which all purine nucleotides are made).

Metabolite and Gene Expression Changes in the Folate-Restricted Animals Consistent with a Reduced Anabolism

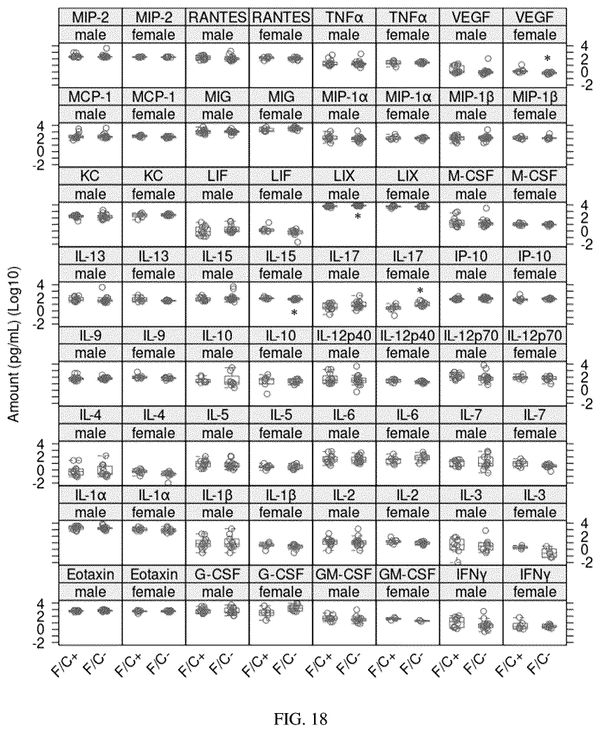

To gauge immune function, the levels of 32 serum cytokines were measured at the time of euthanasia, at 120 weeks of age (FIG. 18). The differences were again minimal, and those that passed the p<0.05 threshold (indicated with the red asterisks in FIG. 18) may be due to lower variance in the measured values and not to major changes in the levels of those cytokines, comparing animals of the same sex but on different diets. These apparent changes, based on the Wilcoxon rank sum test, were: lower IL-15 levels in females on the F/C− diet (p=0.0426); higher IL-17 levels in females on the F/C− diet (p=0.0127); lower VEGF levels in females on the F/C− diet (p=0.0237); and higher LIX levels in males on the F/C− diet (p=0.0015).

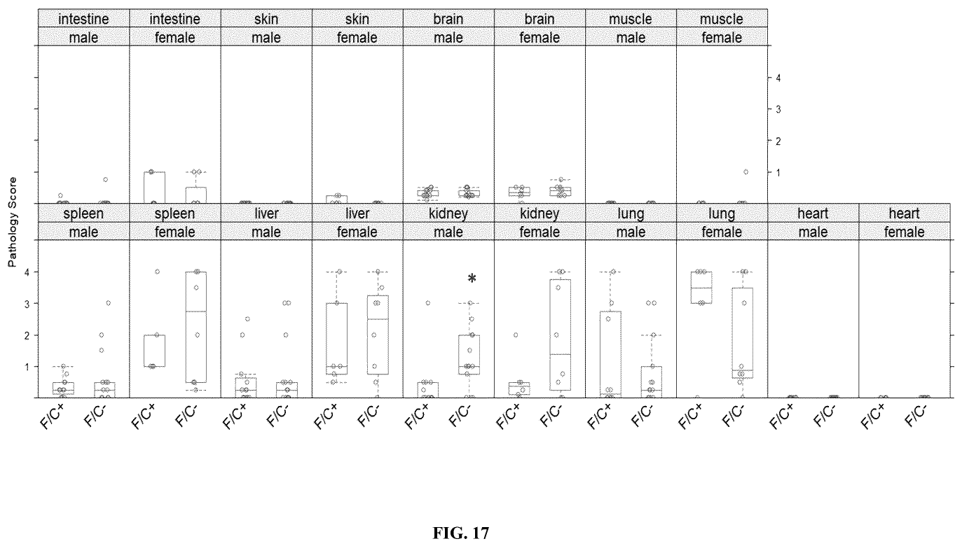

Pathologic evaluation of various organs of the euthanized animals at 120 weeks of age revealed minimal effects on overall disease burden, other than an apparent increase in kidney abnormalities in male animals on the F/C− diet (FIG. 17). Nearly all the mice had some degree/number of perivascular lymphoplasmacytic nodules (Russell bodies), frequently with immunoglobulin aggregates in the ER of plasma cells in the spleen, liver, kidney, and lung. These nodules were higher in number and larger (although only significant in male kidneys) in mice on the F/C− diet, particularly females. This lesion is compatible with but not diagnostic for autoimmune diseases such as Systemic Lupus Erythematosus in aging women. It is noted, however, that although the levels of the pro-inflammatory IL-17 were elevated in female mice as well, no leukocytosis was detected.

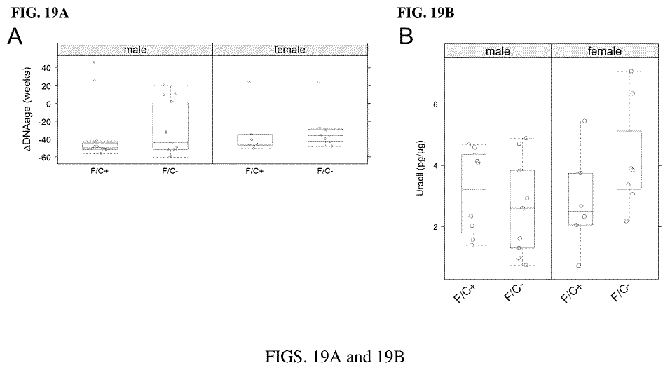

Next, signatures associated with genomic instability were measured. Based on targeted bisulfite sequencing at >1,000 loci, there were no changes in the DNA methylation levels from liver samples collected at 120 weeks of age (FIG. 19A). This analysis yielded an estimate of the DNA methylation age (shown on the y-axis in FIG. 19A), which was not different among the sexes and diet groups. The negative values in all groups we examined reflect comparisons with the expected standard values for the strain C57BL/6J and liver tissue we examined, making all our groups appear “younger” than expected based on their DNA methylation profile for unknown reasons. These data suggest that limiting folate late in life did not lead to significant epigenetic changes. It was also found that uracil misincorporation was not significantly elevated in liver samples collected at 120 weeks of age from mice on the F/C− diet (FIG. 19B). Uracil misincorporation into the genome may reflect limited folate availability for DNA synthesis (i.e., if folate is available, then thymidylate synthesis is unperturbed, and uracil is not expected to be incorporated into the DNA). Hence, limiting folate late in life does not lead to genomic instability in mouse liver, though uracil levels may vary by tissue in other systems of folate limitation.

Broad molecular pathway changes associated with folate restriction late in life were explored next. First, amino acid levels in the sera of all remaining animals were measured, before they were euthanized. Serum glutamine levels were markedly elevated (˜3-fold) in male mice on the folate-limited diet (FIG. 16A). No other significant changes were observed in serum amino acid levels.

Second, the levels of ˜600 metabolites from liver samples of all mice that were alive at 120 weeks of age by mass spectrometry were measured. In male animals, the metabolite with the lowest relative abundance in folate-limited animals was IMP. Serine had the highest relative abundance (FIG. 16B). These results are consistent with reduced 1C metabolism because serine is the primary 1C input (FIG. 2A). The purine nucleotide IMP is a major output (FIG. 2A). Among 589 metabolites detected and assigned, pathway enrichment with the MetaboAnalyst platform was sought. There were no significantly overrepresented pathways in either male or female folate-limited animals. In females, pyrimidine metabolism may be under-represented (p=0.00103, FDR=0.101). In males, metabolites associated with the pentose phosphate pathway, methionine metabolism, and Warburg effect pathways were significantly underrepresented in mice on the F/C− diet (FIG. 16C). These results fit a view of 1C metabolism as a platform that primarily allocates resources for anabolic, biosynthetic pathways. Reducing the output of these pathways late in life is known to promote longevity in other settings.

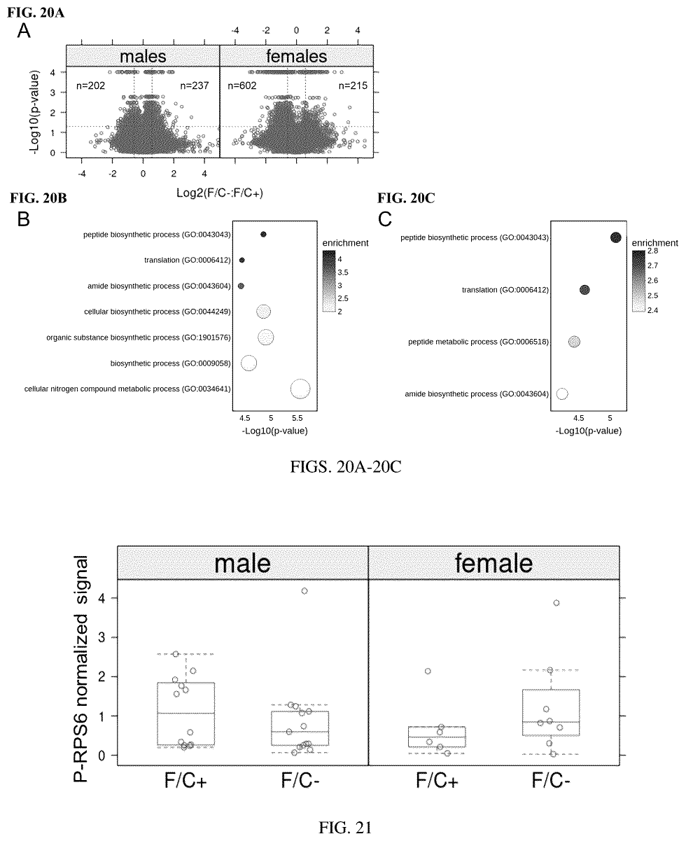

Third, to gauge if and how a folate-limited diet late in life impacts gene expression, transcript steady-state levels of liver tissue with RNAseq were measured. extensive transcriptome remodeling was not observed in response to the folate-limited diet (FIGS. 20A to 20C). Less than 5% of the 15,000-20,000 transcripts that entered the analysis changed in abundance significantly (>1.5-fold, p<0.05; see FIG. 20A). Among the transcripts over-expressed in mice on the F/C-diet, there was no significant enrichment of any gene ontology biological process. On the other hand, among under-expressed transcripts, significant enrichment of transcripts encoding gene products involved in protein synthesis were seen in both male (FIG. 20B) and female animals (FIG. 20C). The enrichment was stronger in males (>3-fold) than in females (˜2-3-fold) but significant in both sexes (FDR<0.05). These results agree with the metabolite analyses (see FIG. 16C), arguing again that a folate-limited diet late in life induces a state of lower anabolism. As a readout of proliferative signaling, the levels of phosphorylation of ribosomal protein S6 (RPS6), which is an output of several kinase cascades, including the mTOR pathway were next examined. Immunoblot analysis of liver tissue samples gathered at the time of euthanasia revealed variability in the detected values across individual mice. When examining the male mice, those fed the F/C-diet, on average, had approximately half the amount of phosphorylated RPS6 (P-RPS6) compared to those on the F/C+ diet. However, due to high variability in the measured values, the overall differences in P-RPS6 levels between the two dietary groups did not reach statistical significance (FIG. 21; p>0.05, based on the Wilcoxon rank sum test). Similar results were obtained using antibodies that detect phosphorylation of 4EBP1 at Thr37,46, which is an output of the mTOR pathway (FIG. 22; p>0.05, based on the Wilcoxon rank sum test).

Lastly, IGF-1 levels in the sera of all animals at the endpoint of euthanasia using an ELISA-based assay were measured. The insulin/insulin-like growth factor pathway plays critical roles in growth control and longevity, mirroring the effects of the mTOR pathway. Female mice on the folate-limited diet had ˜40% lower IGF-1 levels than their counterparts on the folate-replete diet (FIG. 23; p=0.028, based on the Wilcoxon rank sum test). These data are again consistent with the notion that a folate-limited diet late in life may induce a state of lower cell growth and anabolism.

### Example 7

### Selection and Synthesis of Folate Uptake Inhibitors

### Selection of Folate Uptake Inhibitors

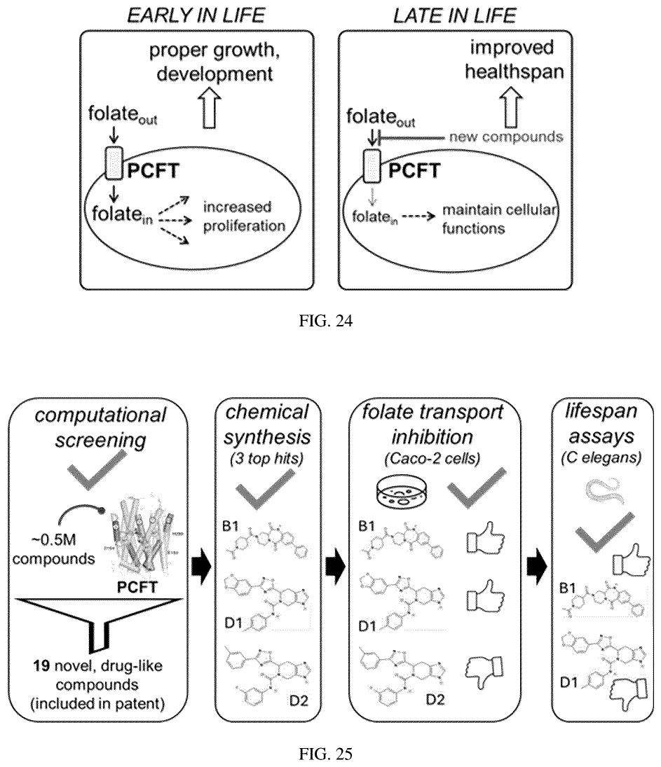

The instant example provides for the selection and analysis of compounds that may not affect the benefits of folate in youth but offer a way to enhance healthspan in older adults by mimicking a low-folate diet through selectively blocking the folate transporter (PCFT) (FIG. 24).

In-silico pharmacology was performed of SLC46A1 including parameter set-up, compounds screening, data collection, and reporting. From a compound library. 2D structures were converted to a 3D structure, and MMFF94S Energy Minimization was performed. Next, the data of the compounds was converted into a pdbqt file in preparation for screening. Half a million compounds were docked computationally to the binding site of SLC46A1 (PDB: 7BC7) (FIG. 25). Among 19 candidates with excellent properties (based on predicted scores of pharmacokinetics, drug-likeness, and medicinal chemistry friendliness of small molecules), three compounds (MPOL B1, MPOL D1, and MPOL D2) were synthesized.

### Synthesis of Folate Uptake Inhibitors

The following examples are offered to illustrate but not to limit the disclosure. One of skill in the art will recognize that the following synthetic reactions and schemes may be modified by choice of suitable starting materials and reagents in order to access other compounds of Formula I, Ia, II, IIa, III, IIIa, IV, IVa, V, or Va.

The proposed targets can be prepared via the conventional chemistry or following the general schemes as shown below.

### Scheme 1: Preparation of 2-(1-acetylpiperidine-4-carbonyl)-8-phenyl-1,3,4,12a-tetrahydrobenzo[e]pyrazino[1,2-a][1,4]diazepine-6,12 (2H,11H)-dione (MPOL_B_1)

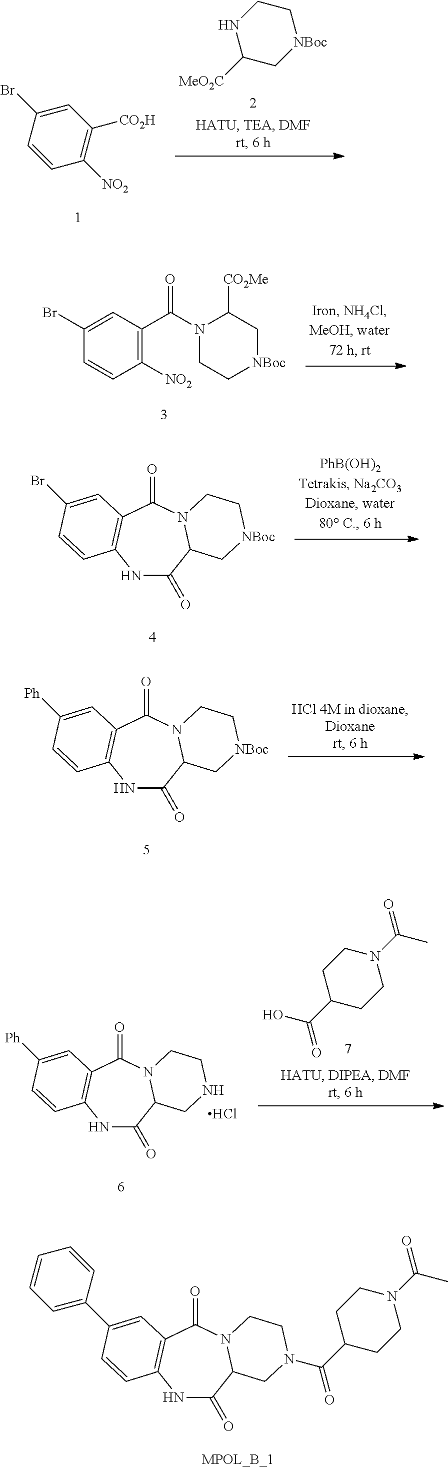

Step 1: The reaction was performed in anhydrous conditions and monitored by LCMS.

To a solution of 1 (1 eq., 500 mg, 2.03 mmol) in DMF (21.0 mL) was added HATU (1.2 eq., 927 mg, 2.44 mmol) and Et₃N (3 eq., 617 mg, 0.85 mL, 6.10 mmol). The reaction mixture was stirred at room temperature for 30 min. Then, 2 (1 eq., 496 mg, 2.03 mmol) was added. The mixture was stirred at 25° C. for 6 hours. The residue was diluted with DCM, washed twice with water. The combined aqueous phases were extracted with DCM again. The combined organic phases were dried over Na₂SO₄ and concentrated under reduced pressure to give 1.0 g of a yellowish solid crude. The crude was purified by reverse phase preparative LC (C18_IR_50SI_F0080, 120 g (solid loading (C18), mobile phase gradient EAU+0.2 wt % NH₄HCO₃/ACN from 10/90 to 90/10). The fractions containing compound were combined and evaporated under reduced pressure to afford 3 (500 mg, 1.06 mmol, 53%).

Step 2: To a solution of 3 (1 eq., 400 mg, 0.85 mmol) in MeOH (5.60 mL) and H₂O (2.41 mL) was added iron (10 eq., 473 mg, 8.47 mmol) and NH₄Cl (0.05 eq., 2.27 mg, 0.042 mmol). The reaction mixture was stirred at room temperature for 3 days.

The mixture was filtrated over celite pad then evaporated in vacuo to afford 4 (320 mg, 0.78 mmol, 93%).

Step 3: The reaction was performed in anhydrous conditions under nitrogen atmosphere and monitored by LCMS and TLC.

To a solution of 4 (1 eq., 300 mg, 0.73 mmol) in dioxane (1.5 mL) and H₂O (1.5 mL) was added Tetrakis(triphenylphosphine)palladium(0) (0.05 eq., 42.25 mg, 0.037 mmol) and Na₂CO₃ (4 eq., 310 mg, 2.92 mmol). The reaction mixture was stirred at room temperature for 30 min. Then, phenylboronic acid (1.1 eq., 98.0 mg, 0.80 mmol) was added. The mixture was heated at 80° C. for 6 hours.

The residue was diluted with DCM, washed twice with water. The combined aqueous phases were extracted with DCM again. The combined organic phases were dried over Na₂SO₄ and concentrated to give 300 mg of crude. The crude was purified by normal phase preparative LC (biotage, Column interchim, IR_50SI_F0080, cyclohexane, Ethyl acetate from 10/90 to 90/10). The fractions containing compound were combined and evaporated under reduced pressure to afford 5 (200 mg, 0.49 mmol, 67%).

Step 4: To a solution of 5 (3.40 eq., 200 mg, 0.49 mmol) in dioxane (2.0 mL) was added HCl in Dioxane (4 M/10 eq., 0.36 mL, 1.44 mmol) The mixture was stirred at 25° C. for 6 hours. The reaction mixture was concentrated in vacuo then the crude was triturated with Et₂O then filtered to afford 6 (150 mg, 0.49 mmol, 100%).

Step 5: The reaction was performed in anhydrous conditions under nitrogen atmosphere and monitored by LCMS and TLC.

To a solution of 6 (1 eq., 150 mg, 0.44 mmol) in DMF (4.50 mL) was added HATU (1.2 eq., 200 mg, 0.52 mmol) and Et₃N (3 eq., 132 mg, 0.18 mL, 1.31 mmol). The reaction mixture was stirred at room temperature for 30 min. Then, 7 (1 eq., 75.0 mg, 0.44 mmol) was added. The mixture was heated at 25° C. for 6 hours. The residue was diluted with DCM, washed twice with water. The combined aqueous phases were extracted with DCM again. The combined organic phases were dried with Na₂SO₄ and concentrated to give a 100 mg of crude. The crude was purified by reverse phase preparative LC (YMC_C18_40G_25 um, (solid loading (C18), mobile phase gradient H₂O (0.2 wt % NH₄HCO₃)/CAN from 90/10 to 00/100). The fractions containing compound were combined and evaporated under reduced pressure to afford 60 mg. Then, the 60 mg was triturated with DCM and Et₂O to afford MPOL_B_1 (20.0 mg, 0.043 mmol, 10%) as a white powder.

### Scheme 2: Preparation of 6-(3-(benzo[d][1,3]dioxol-5-yl)-1,2,4-oxadiazol-5-yl)-N-(p-tolyl)-3,4,6,7-tetrahydro-5H-imidazo[4,5-c]pyridine-5-carboxamide (MPOL_D_1)

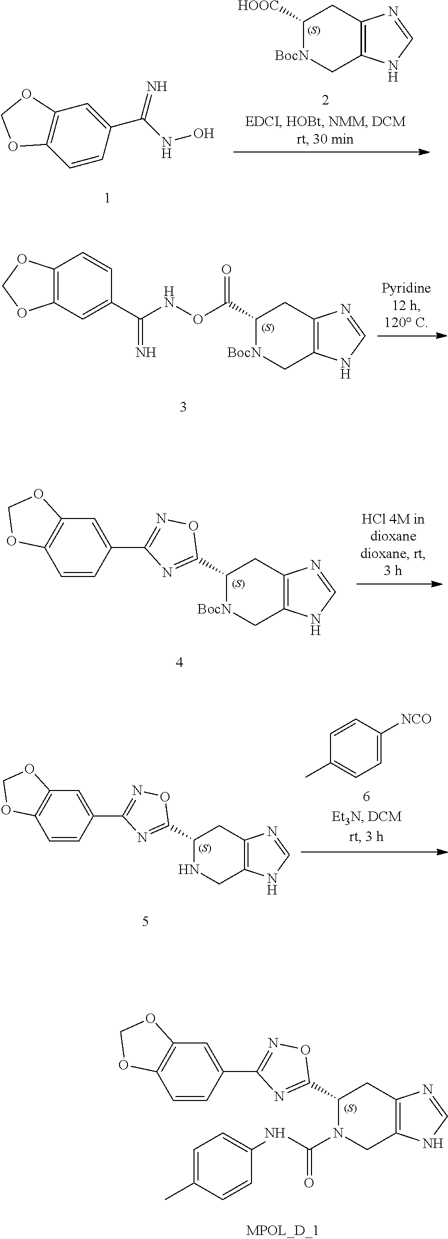

Step 1: The reaction was performed in anhydrous conditions under nitrogen atmosphere and monitored by LCMS and TLC.

To a solution of 1 (1 eq., 200 mg, 1.11 mmol) in DCM was added EDCI (1.3 eq., 224 mg, 1.44 mmol) and HOBt (2 eq., 300 mg, 2.22 mmol). The reaction mixture was stirred at room temperature for 30 min. Then, 2 (0.56 eq., 167 mg, 0.62 mmol) was added. The mixture was heated at 25° C. for 6 hours. The residue was diluted with DCM, washed twice with water. The combined aqueous phases were extracted with DCM again. The combined organic phases were dried over Na₂SO₄ and concentrated to give 3 (100 mg, 0.23 mmol, 37%).

Step 2: The reaction was performed in anhydrous conditions under nitrogen atmosphere and monitored by LCMS and TLC.

A solution of 3 (100 mg, 0.23 mmol) in pyridine (100 mL) was stirred at 120° C. for 12 hours. The reaction mixture was cooled to room temperature and evaporated in vacuo then purified by normal phase preparative LC (biotage, Column interchim, IR_50SI_F0040, solid loading (SiO₂), mobile phase gradient Cyclohexane/Ethyl acetate from 10:90 to 90:10). The fractions containing compound were combined and evaporated under reduced pressure to afford 4 (50.0 mg, 0.12 mmol, 52%).

Step 3: To a solution of 4 (1 eq., 50.0 mg, 0.12 mmol) in dioxane (0.31 mL) was added HCl in dioxane (4 M/6 eq., 0.18 mL, 0.73 mmol) at 25° C. for 3 hours. The reaction mixture was concentrated under reduced pressure to give 5 (79.0 mg, 0.96 mmol, 30%).

Step 4: The reaction was performed in anhydrous conditions under nitrogen atmosphere and monitored by LCMS and TLC.

To a solution of 5 (1 eq., 30 mg, 0.096 mmol) and 6 (1 eq., 12.8 mg, 0.096 mmol) in DCM (0.310 mL) was added Et₃N (3 eq., 29.26 mg, 0.04 mL, 0.29 mmol) at 25° C. for 3 hours. The crude was purified by reverse phase preparative LC (YMC_C18_40g_25 um, (solid loading (C18), mobile phase gradient H₂O (0.2 wt % NH₄CO₃)/ACN 90/10 to 00/100). The fractions containing compound were combined and evaporated under reduced pressure to afford MPOL_D_1 (10.0 mg, 0.022 mmol, 23%) as a white powder.

### Scheme 3: Preparation of N-(3-fluorophenyl)-6-(3-(m-tolyl)-1,2,4-oxadiazol-5-yl)-3,4,6,7-tetrahydro-5H-imidazo[4,5-c]pyridine-5-carboxamide (MPOL_D_2)

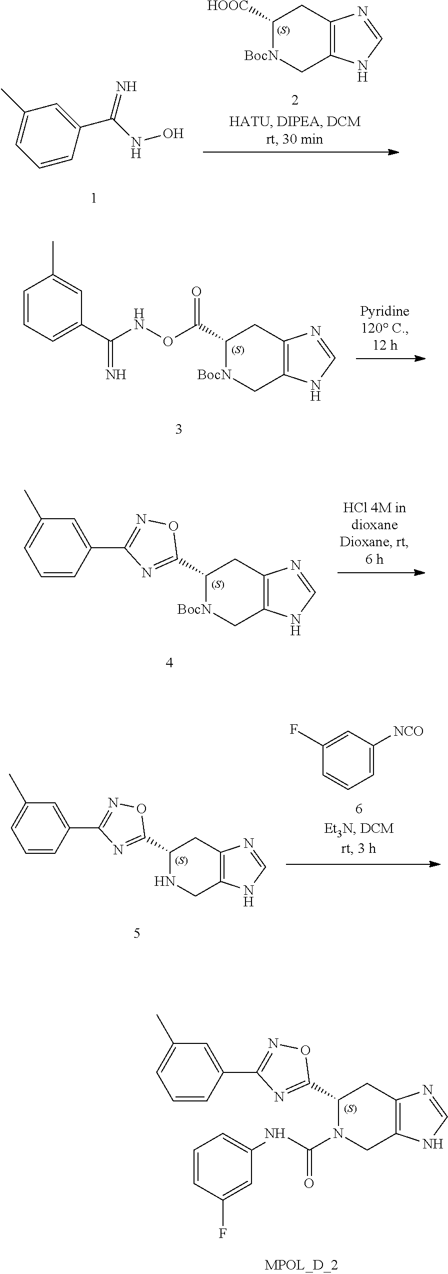

Step 1: The reaction was performed in anhydrous conditions under nitrogen atmosphere and monitored by LCMS and TLC.

To a solution of 1 (1 eq., 169 mg, 1.12 mmol) in DCM was added HATU (1.3 eq., 556 mg, 1.46 mmol) and DIPEA (3 eq., 435 mg, 0.59 mL, 3.37 mmol). The reaction mixture was stirred at room temperature for 30 min, then 2 (1 eq., 300 mg, 1.12 mmol) was added. The mixture was heated at 25° C. for 18 hours. The solvent was evaporated in vacuo to afford 3 (258 mg, 0.65 mmol, 58%).

Step 2: The reaction was performed in anhydrous conditions under nitrogen atmosphere and monitored by LCMS and TLC.

A solution of 3 (1.38 eq., 550 mg, 0.65 mmol) in pyridine (0.80 mL) was stirred at 120° C. for 12 hours. The reaction mixture was cooled to room temperature and evaporated in vacuo to afford 4 (232 mg, 0.61 mmol, 52%).

Step 3: To a solution of 4 (1.07 eq., 400 mg, 0.608 mmol) in dioxane (1.20 mL) was added HCl in dioxane (4M/10 eq., 1.42 mL, 5.69 mmol) The mixture was stirred at 25° C. for 6 hours. The reaction mixture was concentrated in vacuo then the crude was triturated with Et₂O (2×5.0 mL) then filtered to afford 5 (99 mg, 0.31 mmol, 51.22%).

Step 4: The reaction was performed in anhydrous conditions under nitrogen atmosphere and monitored by LCMS and TLC.

To a solution of 5 (1 eq., 99 mg, 0.31 mmol) and 6 (1 eq., 43.0 mg, 0.31 mmol) in DCM (4.50 mL) was added Et3N (10.0 eq., 0.44 ml, 3.15 mmol), the reaction mixture was stirred at room temperature for 3 h then concentrated in vacuo to afford 75 mg of crude. The crude was purified by reverse phase chromatography (YMC_C18_40G_25 um (solid loading (C18)), mobile phase gradient H₂O (0.2 wt % NH₄CO₃)/ACN: from 70:30 to 00:100 over 15 VC). Fractions containing product were collected and evaporated in vacuo then triturated with Et₂O to afford MPOL_D_2 (50.0 mg, 0.12 mmol, 38%).

The following compounds were prepared according to the schemes and methods above:

| Ex. # | Structure | LC/MS (m/z) |
|---|---|---|
| MPOL_B_1 | 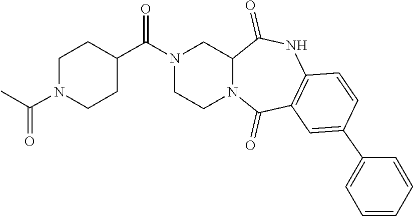 [SMILES: CC(=O)N1CCC(C(=O)N2CCN3C(=O)c4cc(-c5ccccc5)ccc4NC(=O)C3C2)CC1] | 461.2 [M + H]⁺ 459.1 [M − H]⁻ |
| MPOL_D_1 | 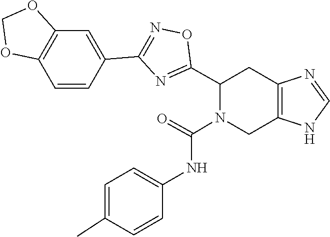 [SMILES: Cc1ccc(NC(=O)N2Cc3[nH]cnc3CC2c2nc(-c3ccc4c(c3)OCO4)no2)cc1] | 445.1 [M + H]⁺ 443.1 [M − H]⁻ |
| MPOL_D_2 | 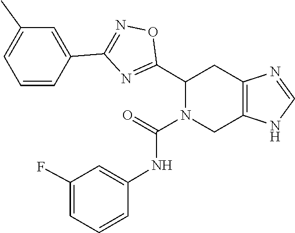 [SMILES: Cc1cccc(-c2noc(C3Cc4nc[nH]c4CN3C(=O)Nc3cccc(F)c3)n2)c1] | 419.1 [M + H]⁺ |

### Example 8

### In Vitro Assessment of Folate Uptake Transporter Inhibition by 3 Compounds in Caco2 Cell Line

### Preparation of Cell Plate.

Cell plates were prepared using Caco-2 cell culture medium consisting of Dulbecco's Modified Eagle's Medium (DMEM) with high glucose and L-glutamine supplemented with: 10% FBS, 1× penicillin-streptomycin mixture and 1×non-essential amino acids (NEAA) and medium was incubated at 37° C., 5% CO2 for 0.5 hour. Plates were prepared for cell seeding. Caco-2 cells from American Type Culture Collection at passage 44 were cultivated in T-75 flasks in a cell culture incubator set at 37° C., 5% CO₂, 95% relative humidity. Cells reached 80-90% confluence before detaching and splitting. Cultivated cells in T-75 flasks were rinsed with 5 mL PBS and aspirated off. Then, 3 mL trypsin/EDTA was added, and cells were incubated at 37° C. for approximately 5 minutes or until the cells detached and floated. Trypsin/EDTA was inactivate by adding excess serum containing medium. Cell suspension was removed and transferred to a conical tube and cells were pelleted by centrifugation at 120×g for 5 minutes. Cells were resuspended in seeding medium at a density of 3×10⁴ cells/well in 24-well plates. The plate was incubated for 8-10 days. The medium was replaced every other day, beginning no sooner than 48 hours after initial plating.

### Preparation of Incubation Solutions.

The transport buffer (HBSS with 10 mM MES, pH 5.5) was prepared by weighing 1.952 g of MES and 0.35 g sodium hydrogen carbonate and adding into 900 mL of pure water followed by sonication to dissolve the content. Following the transfer of 100 mL of 10×HBSS into the solution. The solution was placed on a stirrer, the pH was slowly adjusted with hydrochloric acid to 5.5, and then the solution was filtered to get HBSS solution (with 10 mM MES, pH 5.5).

The [3H]-folic acid (10 nM) substrate solution was prepared using [3H]-folic acid (1 mCi/ml) diluted with pH 5.5 buffer. Next, the [3H]-folic acid (10 nM) substrate solution+test inhibitor (X μM) and the [3H]-folic acid (10 nM) substrate solution+positive control inhibitor (pemetrexed, 50 μM) were prepared. For the solution without inhibitor, DMSO was diluted with corresponding substrate solution to get the working incubation solution.

The pre-incubation solution was prepared by adding the same final concentration of test compound (X μM) and the known inhibitor (pemetrexed, 50 μM) in pH 5.5 buffer. For the solution without inhibitor, DMSO diluted with in pH 5.5 buffer was used as the vehicle control.

### Assay Procedures.

When the 8-10 day Caco-2 cultured cells reached confluence and were differentiated, they were ready to be used for transport studies. The Caco-2 plate was removed from the incubator, and the medium was removed from the 24-well Plates. Next, cells were washed 3 times with 300 μL of pre-warmed pH 5.5 buffer. Then, 300 μL of pre-incubation solution was added into each well and the plate was placed at 37° C. for 30 minutes. The assay was performed in duplicate. After 30 minutes pre-incubation, the pre-incubation solution was aspirated, and 300 μL of pre-warmed working incubation solution was added to start the transporter assay. After 6 minutes incubation, cells were washed by quickly adding/aspirating 300 μL of ice-cold pH 5.5 buffer 4 times. The pH 5.5 buffer was removed and 300 μL of pure water was added into each well. Freeze-thaw in liquid nitrogen and 37° C. incubator was repeated for 3 times to lyse cells. After, centrifugation at 3,300 g was performed for 30 seconds. Next, 50 μL of supernatant was transferred to Isoplate-96 Microplate. 200 μL of scintillation fluid was added to the 50 μL sample and radioactivity was measured by liquid scintillation counting in a MicroBeta 2 scintillation counter for 1 min/sample.

A BCA Kit was used to determine the protein content for each well. Bovine serum albumin at 2, 1, 0.5, 0.25, 0.125, 0.0625, 0.0313, 0.0156, 0.00781 and 0.00391 mg/mL was prepared in Phosphate Buffer Saline (PBS, pH 7.4). 10 μL of supernatant was mixed from the previous step with the same volume of PBS. 20 μL of standard or test sample was added into 96-well clear plate and mixed with 200 μL working reagent. After 30 minutes incubation at 37° C., the absorbance at 562 nm was measured on the microplate reader.

### Data Analysis.

All calculations were carried out using Microsoft Excel. Calculated conc (pmol) were determined from CCPM. Uptake rate, in unit of pmol/mg protein/min, was calculated using the following equation: Uptake rate=Conc./(Protein×Time); Conc. is concentration of drug in cell lysate (nM), Protein is the protein concentration of cell lysate (mg/mL), and Time is the incubation time (minute). The % Percentage transport was calculated using the following equation: Percentage Transported (%)=R(+inhibitor)/R(vehicle control)*100; R(+inhibitor) stands for the uptake rate of substrate in the presence of test compound or positive control inhibitor, and R (vehicle control) stands for uptake rate of substrate in the vehicle control. Percent inhibition (%) was calculated using the following equation: Percentage Inhibition (%)=100-Percentage Transported (%). Note: The compound was considered as a potential substrate of a particular transporter when the uptake ratio (without inhibitor/with positive inhibitor control) was greater than 2, and more than 50% inhibition on uptake ratio was found in the presence of the corresponding inhibitor, compared to the presence of vehicle control.

As shown in Table 1, compound MPOL_B_1 inhibited folate transport at 42.29%; Compound MPOL_D_1 at 25.5%; while compound MPOL_D_2 promoted folate transport.

TABLE 1
Folate Uptake Transporter Inhibition in Caco2 Cell Line

| Compound | | Test Conc. | Percentage |
|---|---|---|---|
| Number | Compound ID | (μM) | inhibition (%) |
| Substrate | Folic acid [3,5,7,9-3H] | 0.01 | — |
| | sodium salt | | |
| Inhibitor | Pemetrexed | 50 | 62.41 |
| 1 | MPOL_D_1 (D1) | 50 | 25.25 |
| 2 | MPOL_D_2 (D2) | 50 | −18.67 |
| 3 | MPOL_B_1 (B1) | 50 | 42.29 |

### Example 9

### In Vitro Assessment of Folate Uptake Transporter Substrate by 3 Compounds in Caco2 Cell Line

### Preparation of Cell Plate.

Cell Plates were Prepared According to the Methods of Example 8 Using Caco-2 Cells from American Type Culture Collection at Passage 37.

### Preparation of Incubation Solutions.

The transport buffer (HBSS with 10 mM MES, pH 5.5) was prepared by weighing 1.952 g of MES and 0.35 g sodium hydrogen carbonate and adding into 900 mL of pure water followed by sonication to dissolve the content. Following the transfer of 100 mL of 10×HBSS into the solution. The solution was placed on a stirrer, the pH was slowly adjusted with hydrochloric acid to 5.5, and then the solution was filtered to get HBSS solution (with 10 mM MES, pH 5.5).

The test compounds and folic acid (1 μM) substrate solutions were prepared by diluting with pH 5.5 buffer. Next, the test compounds and folic acid (1 μM) substrate solution+positive control inhibitor (pemetrexed, 50 μM) were prepared. For the solution without inhibitor, DMSO was diluted with corresponding substrate solution to get the working incubation solution.

For the solution with inhibitor, the pre-incubation solution was prepared by adding the positive control inhibitor (pemetrexed, 50 μM) in pH 5.5 buffer. For the solution without inhibitor, DMSO was diluted with pH 5.5 buffer and used as the vehicle control.

### Assay Procedures.

When the 8-10 day Caco-2 cultured cells reached confluence and were differentiated, they were ready to be used for transport studies. The Caco-2 plate was removed from the incubator, and the medium was removed from the 24-well Plates. Next, cells were washed 3 times with 300 μL of pre-warmed pH 5.5 buffer. Then, 300 μL of pre-incubation solution was added into each well and the plate was placed at 37° C. for 30 minutes. The assay was performed in duplicate. After 30 minutes pre-incubation, the pre-incubation solution was aspirated, and 300 μL of pre-warmed working incubation solution was added to start the transporter assay. After 6 minutes incubation, cells were washed by quickly adding/aspirating 300 μL of ice-cold pH 5.5 buffer 4 times. The pH 5.5 buffer was removed and 300 μL of pure water was added into each well. Freeze-thaw in liquid nitrogen and 37° C. incubator was repeated for 3 times to lyse cells. After, centrifugation at 3,300 g was performed for 30 seconds. Next, 50 μL of supernatant was transferred to 200 μL methanol or acetonitrile containing internal standard and centrifuged at 3,300 g for 30 minutes. 200 μL aliquots of the supernatants were used for LC-MS/MS analysis. Standards were treated the same as samples.

A BCA Kit was used to determine the protein content for each well. Bovine serum albumin at 2, 1, 0.5, 0.25, 0.125, 0.0625, 0.0313, 0.0156, 0.00781 and 0.00391 mg/mL was prepared in Phosphate Buffer Saline (PBS, pH 7.4). 10 μL of supernatant was mixed from the previous step with the same volume of PBS. 20 μL of standard or test sample was added into 96-well clear plate and mixed with 200 μL working reagent. After 30 minutes incubation at 37° C., the absorbance at 562 nm was measured on the microplate reader.

Standard-curves: prepared 200 μM DMSO Folic acid solution, samples of 1000 nm, 300 nm, 100 nm, 30 nM, and 10 nM prepared using a PH 5.5 buffer, and a PH 5.5 buffer containing 50 μM of pemetrexed, respectively.

### Data Analysis

All calculations were carried out using Microsoft Excel. Peak areas were determined from extracted ion chromatograms. Uptake rate, in unit of pmol/mg protein/min, is calculated using the following equation: Uptake rate=Conc./(Protein×Time); Conc. is concentration of drug in cell lysate (nM), Protein is the protein concentration of cell lysate (mg/mL), and Time is the incubation time (minute). Uptake ratio is calculated using the following equation: Uptake ratio=Uptake rate without inhibitor/Uptake rate with inhibitor. Note: The compound was considered as a potential substrate of a particular transporter when the uptake ratio was greater than 2.

As shown in Table 2, the compounds tested enter cells to varying degrees. MPOL_D_1 and MPOL_D_2 are highly permeable. Compound MPOL_B_1 is the least permeable. In all three cases, cell permeability is not through the folate transporter, because their transport is not inhibited by pemetrexed (an inhibitor of the folate transporter, used as a control here).

TABLE 2
Substrate of Folate Uptake Transporter in Caco2 Cells

| | | | | Uptake Rate | |
|---|---|---|---|---|---|
| Compound | | Test Conc. | | (pmol/mg | Uptake |
| Number | Compound ID | (μM) | inhibitor | protein/min) | Ratio_{(-Inhibitor/+Inhibitor)} |
| Positive | Folic acid | 0.01 | Pemetrexed-50 | 0.01 | 2.66 |
| Control | [3,5,7,9-3H]* | | DMSO | 0.02 | |
| 1 | MPOL_D_1 | 1 | Pemetrexed-50 | 9.44 | 1.02 |
| | (D1) | | DMSO | 9.68 | |
| 2 | MPOL_D_2 | 1 | Pemetrexed-50 | 12.97 | 0.86 |
| | (D2) | | DMSO | 11.12 | |
| 3 | MPOL_B_1 | 1 | Pemetrexed-50 | 0.52 | 0.76 |
| | (B1) | | DMSO | 0.39 | |

*Radiolabelled Folic acid [3,5,7,9-3H] was included in the experiment as positive control because the signal of cold folic acid could be significantly interfered with by the co-dosed inhibitor pemetrexed in LC-MS system. LC-MS signal of all test compounds remained unaffected by pemetrexed.

Accordingly, these data in combination with Example 8, indicate that two of the three compounds (B1, D1) are considered to inhibit folate uptake in Caco-2 cells without being transported by PCFT into these cells.

### Example 10

### Healthspan Assays

### Worms Exposed to Compounds

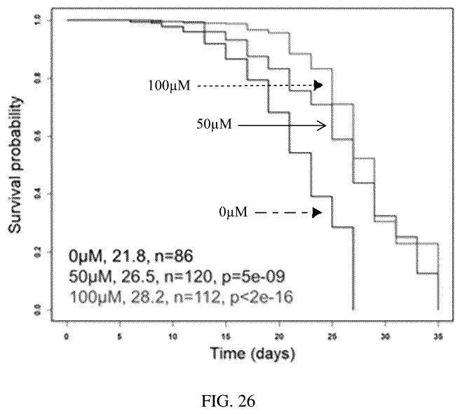

Compound MPOL_B_1 was not toxic in yeast, Caco-2 cells, or worms (all tested at 2-400 μM) and extended worms' maximal and mean lifespan by ˜30% at 50 and 100 μM (FIG. 26). This effect is astounding and >2-3 times the effect of methotrexate using the same assay. Without being bound by any theory, the effect may be correlated to the compound's low toxicity. While worms, like mammals, rely on dietary folate uptake, the worm folate transporter is related but not identical to the mammalian PCFT transporter. Therefore, the effect may be even more significant in mice.

### Mice Exposed to Compounds

Healthspan parameters (e.g., cardiac function, body composition, frailty) can only be measured in live animals in the course of life. For the instant example, mice (strain C57BL/6J) can be evaluated as follows.

First, one animal/dose will be used of one sex (male), using rodents (mouse, rat) and non-rodents (rabbit). The highest dose may be 2000 ppm, in the food pellets, and the lowest 20 ppm. The two lowest effective doses will be re-tested using three animals per test group from both sexes. Twenty animals for each sex per test treatment can be used for evaluating longevity parameters. The compounds of the disclosure can be given continuously, starting at 80 weeks of age, and healthspan behavioral variables, including a validated frailty index, can be measures. Molecular tests can include folate and amino acid levels in sera and a DNA methylation aging clock assay.

The instant example can be performed using one or more of the exemplary procedures provided in Examples 4, 5, or 6.

1. A compound of Formula IV: 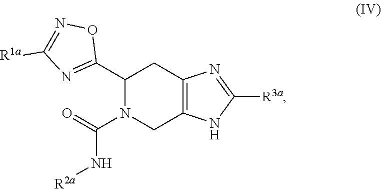
or a pharmaceutically acceptable salt thereof, wherein
each of R^{1a} and R^{2a} is independently substituted or unsubstituted carbocyclyl, heterocyclyl, aryl, or heteroaryl; and
R^{3a} is H.

2. The compound of claim 1, wherein substituted is substituted with one or more of deuterium, hydroxyl, hydroxyalkyl, alkoxy, halogen, alkyl, alkenyl, alkynyl, nitro, silyl, acyl, acyloxy, aryl, cycloalkyl, heterocyclyl, amino, aminoalkyl, cyano, haloalkyl, haloalkoxy, haloalkenyl, haloalkynyl, ketone or oxo, carboxy, amide, ester, OCOCH₂O-alkyl, OP(O)(O-alkyl)₂, or CH₂OP(O)(O-alkyl)₂.

3. The compound of claim 1, wherein R^{1a} is unsubstituted 3- to 12-membered heterocyclyl, or 3- to 12-membered heterocyclyl substituted with deuterium, hydroxyl, halo, alkoxy, alkyl, alkenyl, alkynyl, carboxy, ketone, ester, or amide.

4. The compound of claim 1, wherein R^{1a} is unsubstituted phenyl, or phenyl substituted with deuterium, hydroxyl, halo, alkoxy, alkyl, alkenyl, alkynyl, carboxy, ketone, ester, or amide.

5. The compound of claim 1, wherein R^{1a} is

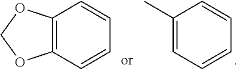

6. The compound of claim 5, wherein R^{2a} is substituted or unsubstituted C₆-C₁₀ aryl.

7. The compound of claim 6, wherein R^{2a} is unsubstituted phenyl, or phenyl substituted with deuterium, hydroxyl, halo, alkoxy, alkyl, alkenyl, alkynyl, carboxy, ketone, ester, or amide.

8. The compound of claim 1, wherein the compound is of Formula IVa: 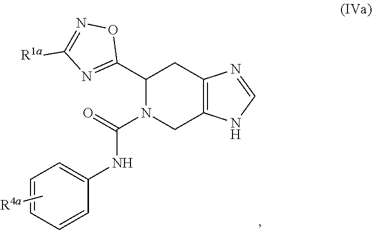
or pharmaceutically acceptable salt thereof, wherein
R^{1a} is as defined; and
R^{4a} is H, deuterium, hydroxyl, halo, alkoxy, alkyl, alkenyl, alkynyl, carboxy, ketone, ester, or amide.

9. The compound of claim 8, wherein R^{4a} is deuterium, hydroxyl, halo, alkoxy, alkyl, alkenyl, alkynyl, carboxy, ketone, ester, or amide.

10. The compound of claim 9, wherein R^{4a} is alkyl or halogen.

11. The compound of claim 10, wherein R^{4a} is methyl or fluoro.

12. The compound of claim 11, wherein the compound is selected from: 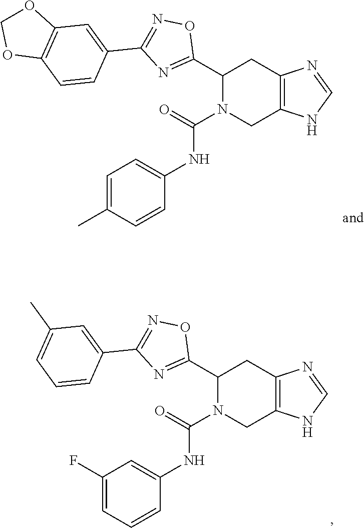 [SMILES: Cc1ccc(NC(=O)N2Cc3[nH]cnc3CC2c2nc(-c3ccc4c(c3)OCO4)no2)cc1 ; Cc1cccc(-c2noc(C3Cc4nc[nH]c4CN3C(=O)Nc3cccc(F)c3)n2)c1]
or a pharmaceutically acceptable salt thereof.

13. A compound selected from: 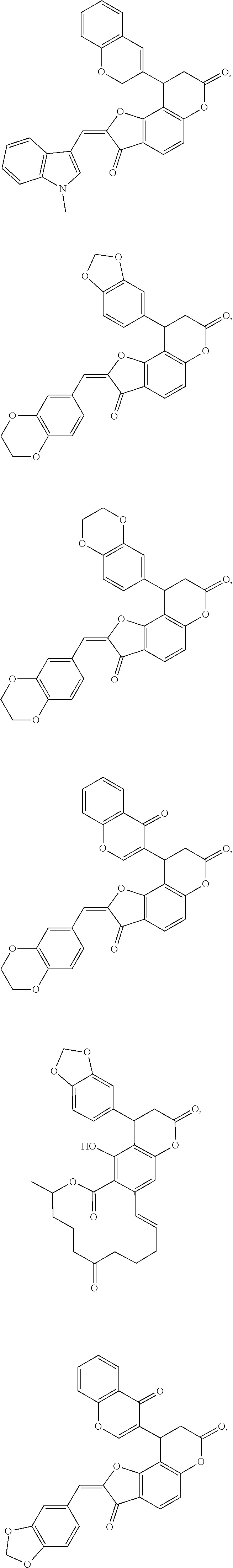 [SMILES: Cn1cc(/C=C2/Oc3c(ccc4c3C(C3=Cc5ccccc5OC3)CC(=O)O4)C2=O)c2ccccc21 ; O=C1CC(c2ccc3c(c2)OCO3)c2c(ccc3c2O/C(=C/c2ccc4c(c2)OCCO4)C3=O)O1 ; O=C1CC(c2ccc3c(c2)OCCO3)c2c(ccc3c2O/C(=C/c2ccc4c(c2)OCCO4)C3=O)O1 ; O=C1CC(c2coc3ccccc3c2=O)c2c(ccc3c2O/C(=C/c2ccc4c(c2)OCCO4)C3=O)O1 ; CC1CCCC(=O)CCC/C=C/c2cc3c(c(O)c2C(=O)O1)C(c1ccc2c(c1)OCO2)CC(=O)O3 ; O=C1CC(c2coc3ccccc3c2=O)c2c(ccc3c2O/C(=C/c2ccc4c(c2)OCO4)C3=O)O1] 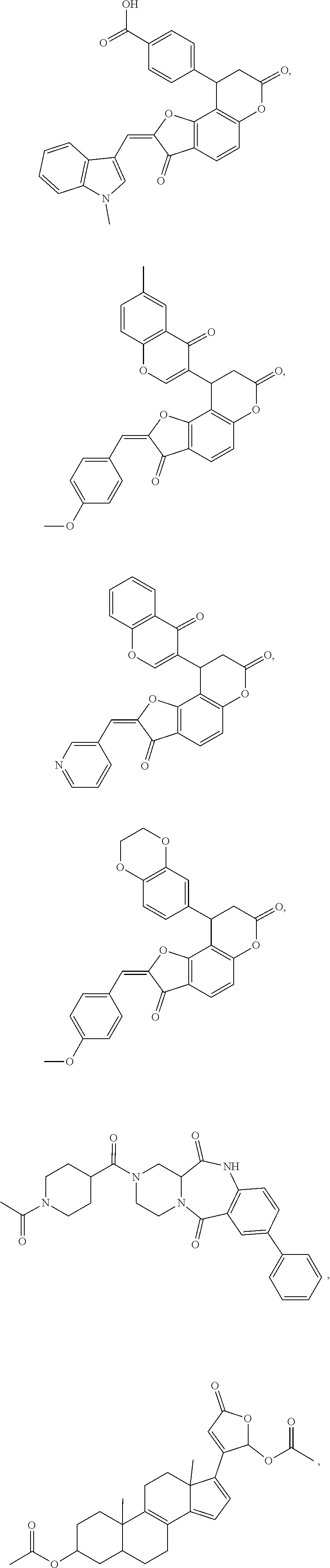 [SMILES: Cn1cc(/C=C2/Oc3c(ccc4c3C(c3ccc(C(=O)O)cc3)CC(=O)O4)C2=O)c2ccccc21 ; COc1ccc(/C=C2/Oc3c(ccc4c3C(c3coc5ccc(C)cc5c3=O)CC(=O)O4)C2=O)cc1 ; O=C1CC(c2coc3ccccc3c2=O)c2c(ccc3c2O/C(=C/c2cccnc2)C3=O)O1 ; COc1ccc(/C=C2/Oc3c(ccc4c3C(c3ccc5c(c3)OCCO5)CC(=O)O4)C2=O)cc1 ; CC(=O)N1CCC(C(=O)N2CCN3C(=O)c4cc(-c5ccccc5)ccc4NC(=O)C3C2)CC1 ; CC(=O)OC1CCC2(C)C3=C(CCC2C1)C1=CC=C(C2=CC(=O)OC2OC(C)=O)C1(C)CC3] 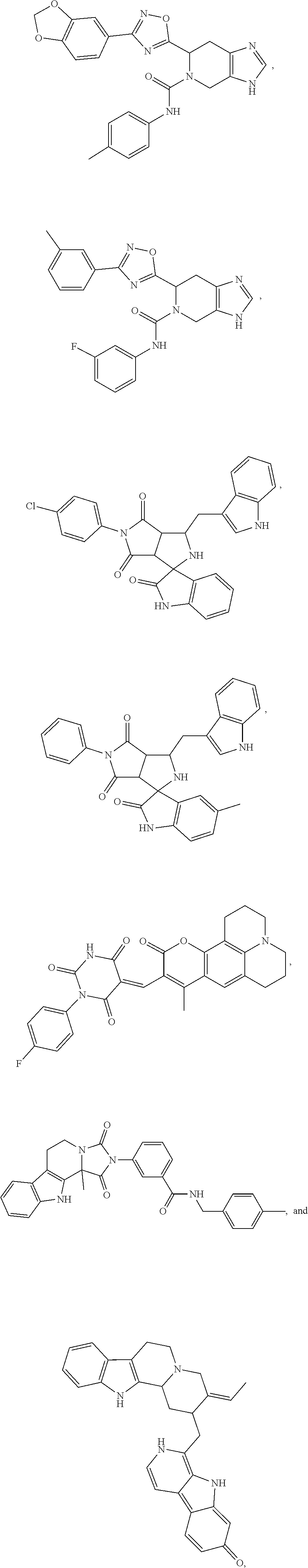
or a pharmaceutically acceptable salt thereof.

14. The compound of claim 13, wherein the compound inhibits a folate receptor.

15. A method of inhibiting folate uptake into a cell of a subject, said method comprising the step of administering a compound according to claim 13 to the subject, wherein the compound inhibits folate uptake into the cell.

16. The method of claim 15, wherein the compound inhibits folate uptake via binding to a folate receptor.

17. The method of claim 16, wherein the folate receptor is a proton-coupled folate transporter (PCFT).

18. The method of claim 16, wherein the folate receptor is a reduced folate receptor (RFC).

19. The method of claim 16, wherein the cell is an intestinal cell.

20. The method of claim 19, wherein the folate uptake is intestinal folate uptake.
